(19) United States
(12) Patent Application Publication
(10) Pub. No.: US 2020/0405742 A1
(43) Pub. Date: Dec. 31, 2020

(54) COMPOSITIONS AND METHODS FOR PAIN AMELIORATION IN PATIENT POPULATION THAT SCORES HIGH ON THE PAIN CATASTROPHIZING SCALE

(71) Applicant: Adynxx Sub, Inc., San Francisco, CA (US)

(72) Inventors: Donald C. MANNING, San Francisco, CA (US); Scott HARRIS, San Francisco, CA (US); Kimberly HEBERT, San Francisco, CA (US); Dina GONZALEZ, San Francisco, CA (US); Julien MAMET, San Francisco, CA (US); William MARTIN, San Francisco, CA (US); Rick ORR, San Francisco, CA (US)

(21) Appl. No.: 16/970,525

(22) PCT Filed: Feb. 25, 2019
(86) PCT No.: PCT/US2019/019401

§ 371 Date: Aug. 17, 2020

Related U.S. Application Data
(60) Provisional application No. 62/634,666, filed on Feb. 23, 2018.

(51) Int. Cl. A61K 31/713; C12N 15/113

(52) U.S. Cl. CPC A61K 31/713; C12N 15/1136

(57) ABSTRACT

The present disclosure relates to method of treating or preventing pain in a patient having a high pain catastrophizing scale (PCS) score, comprising administering an oligonucleotide inhibitor of a transcription factor to said patient.

### CROSS REFERENCE TO RELATED APPLICATIONS

The present PCT Application claims the benefit of priority to U.S. Provisional Application No. 62/634,666, filed on Feb. 23, 2018, the contents of which are hereby incorporated by reference in their entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: ADDY_005_01WO_SeqList_ST25.txt, date recorded: Feb. 20, 2019, file size≈10.6 kilobytes).

### FIELD

The disclosure is directed to pain management. In particular, the disclosure provides a novel method of treating or preventing pain in a particular patient population that is often poorly-responsive to pain treatments.

### BACKGROUND

Pain may be defined as an unpleasant sensory and emotional experience associated with actual or potential tissue damage, or described in terms of such damage. Chronic pain afflicts 40% of the U.S. population and is associated with numerous deleterious medical conditions. Persistent and highly debilitating, chronic pain is generally accompanied by weakness, sleeplessness, a lack of appetite, irritability and depression. Over time, the quality of life is profoundly affected and patients are often incapable of accomplishing the simple tasks of everyday life.

Currently used pain treatments apply a three-step pain ladder which recommends the administration of drugs as follows: non-opioids (e.g., aspirin, acetaminophen, etc.), then, as necessary, mild opioids (e.g., codeine), and finally strong opioids (e.g., morphine). Despite this arsenal of drugs, over 50% of patients with chronic pain are not effectively treated.

The Pain Catastrophizing Scale (PCS) has been used since 1995 and contains 13 items scored from 0 to 4 for a total possible score of 52 (a copy of the scale is provided FIG. 1). Higher scores indicate higher levels of pain-related catastrophizing. The PCS has gone through multiple levels and rounds of validation and has demonstrated a relationship between pain and PCS score in over 100 studies in varying pain conditions.

It has been used clinically to identify individuals with high levels of magnification, rumination and feelings of helplessness regarding their experience of pain. High levels of pain catastrophizing reflected in higher PCS scores have been associated with poor postoperative outcomes including increased pain intensity, increased analgesic use and opioid misuse as well as longer times to achieve postoperative functional targets. Of importance, higher PCS scores have been associated with resistance to standard analgesic interventions. It is commonly reported in the field that a PCS ≥16 or ≥20 can be used as threshold for identifying high scorers on the PCS.

Current pain management strategies that rely on non-opioid analgesics (e.g., acetaminophen and nonsteroidal anti-inflammatory drugs) and/or opioid analgesics are not very effective in patients with higher PCS scores. Thus, pain prevention or pain management treatments directed to patients with higher PCS scores are needed.

### SUMMARY OF THE DISCLOSURE

In some embodiments, provided herein are methods for treating or preventing pain in a patient that has a high pain catastrophizing scale (PCS) score by administering an oligonucleotide inhibitor of a transcription factor. In some embodiments, the patient has a high PCS score is a score of ≥20 or ≥16. In some embodiments, the patient has a PCS score of 16 or greater. In some embodiments, the patient has a PCS score of 20 or greater.

In some embodiments, the oligonucleotide inhibitor is an oligonucleotide decoy comprising one or more transcription factor binding sites. In one embodiment, the transcription factor is Early Growth Response protein 1 (EGR1).

In some embodiments, methods described herein comprise administering an oligonucleotide inhibitor, which is an oligonucleotide decoy, comprising a nucleic acid sequence comprising a sense strand having a sequence selected from SEQ ID NOs: 1-53. In some embodiments, the oligonucleotide decoy comprises an antisense strand having a sequence that is fully complementary to the sequence selected from SEQ ID NOs: 1-53.

In some other embodiments, the oligonucleotide inhibitor administered to a patient is an oligonucleotide decoy comprising a sequence selected from the group consisting of: (a) SEQ ID NOs: 1-53; (b) a sequence that is at least 90% identical to the sequence selected from SEQ ID NOs: 1-53; (c) a sequence that is at least 85% identical to the sequence selected from SEQ ID NOs: 1-53; and (d) a sequence that is at least 80% identical to the sequence selected from SEQ ID NOs: 1-53. In some embodiments, the oligonucleotide inhibitor administered to a patient is an oligonucleotide decoy comprising a sequence selected from the group consisting of: (a) the sequence of SEQ ID NO.: 42; (b) a sequence that is at least 90% identical with SEQ ID NO.: 42; (c) a sequence that is at least 85% identical with SEQ ID NO.: 42; or (d) a sequence that is at least 80% identical with SEQ ID NO.: 42.

In some embodiments, methods described herein comprise administering an oligonucleotide inhibitor, wherein the oligonucleotide inhibitor is an oligonucleotide decoy comprising a nucleic acid sequence comprising a sense strand of 5′-GTATGCGTGGGCGGTGGGCGTAG-3′ (SEQ ID NO: 42). In some embodiments, the oligonucleotide decoy comprises an antisense strand of 3′-CATACGCACCCGCCACCCGCATC-5′. In some embodiments, the oligonucleotide decoy comprises a sense strand comprising the sequence of 5′-GTATGCGTGGGCGGTGGGCGTAG-3′ (SEQ ID NO: 42) and an antisense strand comprising the sequence of 3′-CATACGCACCCGCCACCCGCATC-5′.

In some embodiments, methods described herein comprise administering an oligonucleotide inhibitor, wherein the oligonucleotide inhibitor is brivoligide.

In some embodiments, methods of the present disclosure can be used for treating or preventing perioperative pain in a patient. In some embodiments, methods of the present disclosure can be used for treating or preventing post-operative pain.

In some embodiments, methods of the present disclosure provide a clinically meaningful reduction in pain experienced by the patient. In certain embodiments, the patient may experience a clinically meaningful reduction in pain through at least day 28 post-surgery, through at least day 42 post-surgery, or through at least day 90 post-surgery.

In some embodiments, a patient treated with the methods described herein may experience at least an additional 20% reduction in pain compared to a patient not administered the oligonucleotide inhibitor.

In some embodiments, a patient treated with the methods described herein may experience a clinically meaningful reduction in movement-evoked pain. In these embodiments, the patient may experience a clinically meaningful reduction in movement-evoked pain through at least day 28 post-surgery, through at least day 42 post-surgery, or through at least day 90 post-surgery.

In some embodiments, a patient treated with the methods described herein may experience at least an additional 20% reduction in movement-evoked pain compared to a patient not administered the oligonucleotide inhibitor. In some embodiments, a patient treated with the methods described herein may experience at least an additional 20% reduction in movement-evoked pain through at least day 28 post-surgery, through at least day 42 post-surgery, or through at least day 90 post-surgery, compared to a patient not administered the oligonucleotide inhibitor.

In some embodiments, a patient treated with the methods described herein may experience a statistically or clinically effective reduction in pain at rest. In some embodiments, the patient may experience a clinically meaningful reduction in pain at rest through at least day 28 post-surgery, through at least day 42 post-surgery, or through at least day 90 post-surgery.

In some embodiments, a patient treated with the methods described herein may experience at least an additional 20% reduction in pain at rest compared to a patient not administered the oligonucleotide inhibitor. In some embodiments, a patient treated with the methods described herein may experience at least an additional 20% reduction in pain at rest through at least day 28 post-surgery, through at least day 42 post-surgery, or through at least day 90 post-surgery, compared to a patient not administered the oligonucleotide inhibitor.

In some embodiments, a patient treated with the methods described herein may experience a clinically meaningful reduction in movement-evoked pain from about day 7 post-surgery through at least day 28 post-surgery.

In some embodiments, a patient treated with the methods herein may experience a clinically meaningful reduction in pain at rest from about day 7 post-surgery through at least day 28 post-surgery.

In some embodiments, a patient treated with the methods described herein may experience a clinically meaningful reduction in movement-evoked pain from about day 7 post-surgery through at least day 42 post-surgery.

In some embodiments, a patient treated with the methods described herein may experience a clinically meaningful reduction in pain at rest from about day 7 post-surgery through at least day 42 post-surgery.

In some embodiments, a patient treated with the methods described herein may experience a clinically meaningful reduction in movement-evoked pain from about day 7 post-surgery through at least day 90 post-surgery.

In some embodiments, a patient treated with the methods described herein may experience a clinically meaningful reduction in pain at rest from about day 7 post-surgery through at least day 90 post-surgery.

In some embodiments, a patient treated with the methods described herein may experience at least an additional 20% reduction in movement-evoked pain or pain at rest from about day 7 post-surgery through at least day 28 post-surgery, from about day 7 post-surgery through at least day 42 post-surgery, or from about day 7 post-surgery through at least day 90 post-surgery, compared to a patient not administered the oligonucleotide inhibitor.

In some embodiments, opioid consumption by a patient treated with the methods described herein is reduced from day 0 post-surgery through at least day 90 post-surgery compared to a patient not administered the oligonucleotide inhibitor.

In some embodiments, daily average opioid consumption by a patient treated with the methods described herein is reduced compared to a patient not administered the oligonucleotide inhibitor.

In some embodiments, opioid consumption from day 0 post-surgery through at least day 90 post-surgery by a patient treated with the methods described herein is reduced by an additional 5%, 10%, 15%, 20%, 25%, 30%, 35%, 40%, 45%, or 50%, compared to a patient not administered the oligonucleotide inhibitor. In some embodiments, daily average opioid consumption by a patient treated with the methods described herein is reduced by an additional 5%, 10%, 15%, 20%, 25%, 30%, 35%, 40%, 45%, or 50%, compared to a patient not administered the oligonucleotide inhibitor.

In some embodiments, a patient treated with the methods described herein may experience a reduction in movement-evoked pain, wherein said reduction in pain is at least a 0.5 to 1 point reduction in pain, as measured by an 11 point numerical rating scale, experienced by said patient compared to a patient not administered the oligonucleotide inhibitor.

In some embodiments, a patient treated with the methods described herein may experience a reduction in pain at rest, wherein said reduction in pain is at least a 0.5 to 1 point reduction in pain, as measured by an 11 point numerical rating scale, experienced by said patient as compared to a patient not administered the oligonucleotide inhibitor.

In some embodiments, a patient treated with the methods described herein may experience a reduction in movement-evoked pain from about day 7 post-surgery through at least day 28 post-surgery, wherein said reduction in pain is at least a 0.5 to 1 point reduction in pain, as measured by an 11 point numerical rating scale, experienced by said patient as compared to a patient not administered the oligonucleotide inhibitor.

In some embodiments, a patient treated with the methods described herein may experience a reduction in pain when at rest from about day 7 post-surgery through at least day 28 post-surgery, wherein said reduction in pain is at least a 0.5 to 1 point reduction in pain, as measured by an 11 point numerical rating scale, experienced by said patient as compared to a patient not administered the oligonucleotide inhibitor.

In some embodiments, a patient treated with the methods described herein may experience a reduction in movement-evoked pain from about day 7 post-surgery through at least day 42 post-surgery, wherein said reduction in pain is at least a 0.5 to 1 point reduction in pain, as measured by an 11 point numerical rating scale, experienced by said patient as compared to a patient not administered the oligonucleotide inhibitor.

In some embodiments, a patient treated with the methods described herein may experience a reduction in pain at rest from about day 7 post-surgery through at least day 42 post-surgery, wherein said reduction in pain is at least a 0.5 to 1 point reduction in pain, as measured by an 11 point numerical rating scale, experienced by said patient as compared to a patient not administered the oligonucleotide inhibitor.

In some embodiments, a patient treated with the methods described herein may experience a reduction in movement-evoked pain from about day 7 post-surgery through at least day 90 post-surgery, wherein said reduction in pain is at least a 0.5 to 1 point reduction in pain, as measured by an 11 point numerical rating scale, experienced by said patient as compared to a patient not administered the oligonucleotide inhibitor.

In some embodiments, a patient treated with the methods described herein may experience a reduction in pain at rest from about day 7 post-surgery through at least day 90 post-surgery, wherein said reduction in pain is at least a 0.5 to 1 point reduction in pain, as measured by an 11 point numerical rating scale, experienced by said patient as compared to a patient not administered the oligonucleotide inhibitor.

In some embodiments, a patient treated with the methods described herein may experience a reduction in movement-evoked pain, wherein said reduction in pain is at least an additional 20% reduction in pain, as measured by an 11 point numerical rating scale, experienced by said patient as compared to a patient not administered the oligonucleotide inhibitor.

In some embodiments, a patient treated with the methods described herein may experience a reduction in pain when at rest, wherein said reduction in pain is at least an additional 20% reduction in pain, as measured by an 11 point numerical rating scale, experienced by said patient as compared to a patient not administered the oligonucleotide inhibitor.

In some embodiments, a patient treated with the methods described herein may experience a reduction in movement-evoked pain from about day 7 post-surgery through at least day 28 post-surgery, wherein said reduction in pain is at least an additional 20% reduction in pain, as measured by an 11 point numerical rating scale, experienced by said patient as compared to a patient not administered the oligonucleotide inhibitor.

In some embodiments, a patient treated with the methods described herein may experience a reduction in pain when at rest from about day 7 post-surgery through at least day 28 post-surgery, wherein said reduction in pain is at least an additional 20% reduction in pain, as measured by an 11 point numerical rating scale, experienced by said patient as compared to a patient not administered the oligonucleotide inhibitor.

In some embodiments, a patient treated with the methods described herein may experience a reduction in movement-evoked pain from about day 7 post-surgery through at least day 42 post-surgery, wherein said reduction in pain is at least an additional 20% reduction in pain, as measured by an 11 point numerical rating scale, experienced by said patient as compared to a patient not administered the oligonucleotide inhibitor.

In some embodiments, a patient treated with the methods described herein may experience a reduction in pain at rest from about day 7 post-surgery through at least day 42 post-surgery, wherein said reduction in pain is at least an additional 20% reduction in pain, as measured by an 11 point numerical rating scale, experienced by said patient as compared to a patient not administered the oligonucleotide inhibitor.

In some embodiments, a patient treated with the methods described herein may experience a reduction in movement-evoked pain from about day 7 post-surgery through at least day 90 post-surgery, wherein said reduction in pain is at least an additional 20% reduction in pain, as measured by an 11 point numerical rating scale, experienced by said patient as compared to a patient not administered the oligonucleotide inhibitor.

In some embodiments, a patient treated with the methods described herein may experience a reduction in pain at rest from about day 7 post-surgery through at least day 90 post-surgery, wherein said reduction in pain is at least an additional 20% reduction in pain, as measured by an 11 point numerical rating scale, experienced by said patient as compared to a patient not administered the oligonucleotide inhibitor.

In some embodiments, a patient treated with the methods described herein takes 15 to 30 days less to achieve reduction in pain, movement-evoked pain, or pain at rest, compared to a patient not administered the oligonucleotide inhibitor.

In some embodiments, the oligonucleotide inhibitor is administered to a patient at a concentration of about 110 mg/mL±25%.

In some embodiments, the oligonucleotide inhibitor is administered to a patient at a concentration from about 660 mg/6 mL to less than about 1100 mg/10 mL.

In some embodiments, the oligonucleotide inhibitor is administered to a patient at a concentration of less than about 1100 mg/10 mL.

In some embodiments, the oligonucleotide inhibitor is administered to a patient at a concentration from about 500 mg/5 mL to about 700 mg/7 mL.

In some embodiments, the oligonucleotide inhibitor is administered to a patient at a concentration from about 330 mg/3 mL to about 660 mg/6 mL.

In some embodiments, the oligonucleotide inhibitor is administered to a patient at a concentration of about 660 mg/6 mL±25%.

In some embodiments, the oligonucleotide inhibitor is administered to a patient at a concentration of about 660 mg/6 mL.

In some embodiments, this disclosure provides a method for treating or preventing pain in a patient with a high pain catastrophizing scale score by administering brivoligide to the patient.

In some embodiments, this disclosure provides a method for perioperative pain treatment or prevention in a patient with a high pain catastrophizing scale score by administering brivoligide.

In another embodiment, this disclosure provides a method for treating or preventing pain in a patient with a high pain catastrophizing scale score by administering an oligonucleotide decoy to said patient, wherein the oligonucleotide decoy comprises a nucleic acid sequence comprising a sense strand of 5′-GTATGCGTGGGCGGTGGGCGTAG-3′ and antisense strand of 3′-CATACGCACCCGCCACCCGCATC-5′.

In some embodiments, this disclosure provides a method for treating or preventing pain in a patient, said patient having a high pain catastrophizing scale score by administering an oligonucleotide decoy to said patient, wherein the oligonucleotide decoy comprises SEQ ID NO: 42.

In some embodiments, this disclosure provides a method for treating or preventing pain in a patient, said patient having a high pain catastrophizing scale score by administering an oligonucleotide decoy to said patient, wherein the oligonucleotide decoy has one or more EGR1 transcription factor binding sites.

In some embodiments, this disclosure provides a method for treating or preventing pain in a patient that is a member of a patient population that is often poorly-responsive to pain treatments by administering brivoligide to at least one member of said patient population.

In some embodiments, this disclosure provides a method for perioperative pain treatment or prevention in a patient that is a member of a patient population that is often poorly-responsive to pain treatments by administering brivoligide to at least one member of said patient population.

In some embodiments, this disclosure provides a method for treating or preventing pain in a patient that is a member of a patient population that is often poorly-responsive to pain treatments, comprising: administering an oligonucleotide decoy to at least one member of said patient population, wherein the oligonucleotide decoy comprises a nucleic acid sequence comprising a sense strand of 5′-GTATGCGTGGGCGGTGGGCGTAG-3′ and antisense strand of 3′-CATACGCACCCGCCACCCGCATC-5′.

In some embodiments, this disclosure provides a method for treating or preventing pain in a patient that is a member of a patient population that is often poorly-responsive to pain treatments by administering an oligonucleotide decoy to at least one member of said patient population, wherein the oligonucleotide decoy comprises SEQ ID NO: 42.

In some embodiments, this disclosure provides a method for treating or preventing pain in a patient that is a member of a patient population that is often poorly-responsive to pain treatments by administering an oligonucleotide decoy to at least one member of said patient population, wherein the oligonucleotide decoy has one or more EGR1 transcription factor binding sites.

### BRIEF DESCRIPTION OF THE FIGURES

FIG. 1. Pain Catastrophizing Scale

FIG. 2. PCS distribution in ADYX-004

FIG. 3. ADYX-004 Pain with walking and at rest 7-28 days by baseline PCS (Mean pain rating)

FIG. 4. ADYX-004 Worst pain by baseline PCS (Mean pain rating)

FIG. 5. ADYX-004 Time to achieve NRS ≤3 for worst pain by baseline PCS score

FIG. 6. ADYX-004 Opioid consumption by baseline PCS

FIG. 7. ADYX-004 Daily average opioid consumption by baseline PCS

FIG. 8. ADYX-004 Pain with walking and at rest (weekly) in the PCS ≥20 population (Mean pain rating)

FIG. 9. ADYX-003 Weekly analysis of NRS walk, rest and 90° flexion by baseline PCS ≥20

FIG. 10. Combined ADYX-003 and ADYX-004 Pain at Rest weekly analysis by Baseline PCS (mean NRS)

FIG. 11. Combined ADYX-003 and ADYX-004 Pain at Rest weekly analysis by Baseline PCS (mean NRS)

FIG. 12. Combined ADYX-003 and ADYX-004 Pain with Walking weekly analysis by Baseline PCS (mean NRS).

FIG. 13. ADYX-002 Weekly analysis of NRS walk, rest and 90° flexion by baseline PCS ≥20

### DETAILED DESCRIPTION OF THE DISCLOSURE

Following long-standing patent law convention, the terms “a”, “an”, and “the” refer to “one or more” when used in this application, including the claims, unless clearly indicated otherwise.

Throughout this application, the term “about” is used to indicate that a value includes the standard deviation of error for the device or method being employed to determine the value, e.g. ±10%.

“Acute” refers to a period of time that is shorter than “chronic.” Acute pain is where pain symptoms appear suddenly and do not extend beyond healing of the underlying injury. In embodiments, acute pain can be measured in hours or even days. Thus, the methods and compositions of the disclosure are able to treat acute pain.

“Binding,” as used in the context of transcription factors binding to oligonucleotide decoys, refers to a direct interaction (e.g., non-covalent bonding between the transcription factor and oligonucleotide decoy, including hydrogen-bonding, van der Waals bonding, etc.) between at least one transcription factor and an oligonucleotide decoy. Accordingly, an oligonucleotide that does not bind to a transcription factor does not directly interact with said transcription factor.

“Chronic” refers to a period of time that is longer than “acute.” Chronic pain, unlike acute pain, is a process that lasts for a long period of time. In some embodiments, chronic is a period of time comprising months (e.g., at least 1, 2, 3, 4, 5, 6, 7, 8, 9, 10, 11, and 12 months) or years. In some embodiments, “chronic pain” refers to pain that lasts 3 months or more in a patient. Accordingly, in some embodiments, the methods and compositions of the disclosure are able to treat chronic pain, i.e. pain that lasts 3 months or more.

“Compounds”, in some aspects, refer to oligonucleotide inhibitors of transcription factors. In one aspect, an oligonucleotide inhibitor is a double-stranded oligonucleotide, also referred to herein as an oligonucleotide decoy. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. Compounds may also exist in several tautomeric forms including the enol form, the keto form and mixtures thereof. Accordingly, the chemical structures depicted herein encompass all possible tautomeric forms of compounds. Compounds described herein also include isotopically labeled compounds where one or more atoms have an atomic mass different from the atomic mass conventionally found in nature. Examples of isotopes that may be incorporated into the compounds of the invention include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. All physical forms are equivalent for the uses contemplated herein. Further, it should be understood, when partial structures of the compounds are illustrated, that brackets indicate the point of attachment of the partial structure to the rest of the molecule.

As used herein, the term “effective” (e.g., “an effective amount”) means adequate to accomplish a desired, expected, or intended result. An effective amount can be a therapeutically effective amount. A “therapeutically effective amount” refers to the amount of an active ingredient (e.g. an oligonucleotide decoy) that, when administered to a subject, is sufficient to effect such treatment of a particular disease or condition (e.g. pain). The “therapeutically effective amount” will vary depending on the active ingredient, the disease or condition, the severity of the disease or condition, and the age, weight, etc., of the subject to be treated.

The terms “minimizing,” “inhibiting,” and “reducing,” or any variation of these terms, includes any measurable decrease or complete inhibition or reduction to achieve a desired result. For example, there may be a decrease of 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity compared to normal.

“Modulation of gene expression level” refers to any change in gene expression level, including an induction or activation (e.g., an increase in gene expression), an inhibition or suppression (e.g., a decrease in gene expression), or a stabilization (e.g., prevention of the up-regulation or down-regulation of a gene that ordinarily occurs in response to a stimulus, such as a pain-inducing stimulus).

“Nociceptive signaling” refers to molecular and cellular mechanisms involved in the detection of a noxious stimulus or of a potentially harmful stimulus, which leads to the perception of pain, including neurotransmitter synthesis and release, neurotransmitter-induced signaling, membrane depolarization, and related intra-cellular and inter-cellular signaling events.

An “oligonucleotide inhibitor” refers to any single-stranded or double-stranded, nucleic acid-containing polymer generally less than approximately 200 nucleotides in length. In some embodiments, an oligonucleotide inhibitor can be an oligonucleotide decoy. The term an “oligonucleotide decoy” as used herein refers to a double-stranded, nucleic acid-containing polymer generally less than approximately 200 nucleotides (or 100 base pairs) in length and including, but not limited to: DNA-DNA, RNA-RNA and RNA-DNA hybrids.

The term “oligonucleotide inhibitor” encompasses sequences that include any of the known base analogs of DNA and RNA including, but not limited to, 2,6-diaminopurine, 5-carboxymethylaminomethyl-2-thiouracil, 5-carboxymethylaminomethyluracil, dihydrouracil, inosine, uracil-5-oxyacetic acid, N6-isopentenyladenine, 1-methyladenine, N-uracil-5-oxyacetic acid methylester, queosine, 2-thiocytosine, 5-bromouracil, methylphosphonate, phosphorodithioate, ormacetal, 3′-thioformacetal, nitroxide backbone, sulfone, sulfamate, morpholino derivatives, locked nucleic acid (LNA) derivatives, or peptide nucleic acid (PNA) derivatives. In some embodiments, the oligonucleotide inhibitor is an oligonucleotide decoy composed of two complementary single-stranded oligonucleotides that are annealed together. In other embodiments, the oligonucleotide inhibitor is an oligonucleotide decoy that is composed of one single-stranded oligonucleotide that forms intramolecular base pairs to create a substantially double-stranded structure.

“Pain” refers to an unpleasant sensory and emotional experience that is associated with actual or potential tissue damage or described in such terms. All of the different manifestations and qualities of pain, including mechanical pain (e.g., induced by a mechanical stimulus or by body motion), temperature-induced pain (e.g., pain induced by hot, warm and/or cold temperatures), and chemically-induced pain (e.g., pain induced by a chemical) are included. In certain embodiments, pain is chronic, sub-chronic, acute, or sub-acute. In certain embodiments, pain features hyperalgesia (i.e., an increased sensitivity to a painful stimulus) and/or allodynia (i.e., a painful response to a usually non-painful stimulus). In certain embodiments, pain is pre-existing in a patient. In other embodiments, pain is iatrogenic, induced in a patient (e.g., post-operative pain).

“Pharmaceutically acceptable salt” refers to a salt of a compound, which possesses the desired pharmacological activity of the parent compound. Such salts include, but are not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which a compound of the invention is administered.

“Patient” includes any animal, including birds, mammals, primates, and humans. In particular embodiments, the patient is a human having a high PCS score, such as a score of 20 or greater or a score of 16 or greater on the pain catastrophizing scale (PCS).

“Preventing” or “prevention” refers to (1) a reduction in the risk of acquiring a disease or disorder (e.g., causing at least one of the clinical symptoms of a disease not to develop in a patient that may be exposed to or predisposed to the disease, but does not yet experience or display symptoms of the disease), or (2) a reduction in the likely severity of a symptom associated with a disease or disorder (e.g., reducing the likely severity of at least one of the clinical symptoms of a disease in a patient that may be exposed to or predisposed to the disease, but does not yet experience or display symptoms of the disease).

“Treating” or “treatment” of any condition, disease, or disorder refers, in some embodiments, to ameliorating the condition, disease, or disorder (i.e., arresting or reducing the development of the disease or at least one of the clinical symptoms thereof). In other embodiments “treating” or “treatment” refers to ameliorating at least one physical parameter, which may not be discernible by the patient. In yet other embodiments, “treating” or “treatment” refers to inhibiting the condition, disease, or disorder, either physically, (e.g., stabilization of a discernible symptom), physiologically, (e.g., stabilization of a physical parameter) or both. In yet other embodiments, “treating” or “treatment” refers to delaying the onset of the condition, disease, or disorder.

“Clinically meaningful” means a reduction in pain experienced by a patient taking a treatment of approximately at least an additional 10% compared to a patient not administered the treatment (See, for example, Olsen, M F et al., “Pain relief that matters to patients: systematic review of empirical studies assessing the minimum clinically important difference in acute pain,” BMC Medicine, 2017, 15:35, incorporated by reference herein in its entirety).

“Therapeutically effective amount” means the amount of a compound that, when administered to a patient, is sufficient to effect such treatment of a particular disease or condition. The “therapeutically effective amount” will vary depending on the compound, the disease, the severity of the disease, and the age, weight, etc., of the patient to be treated. In certain aspects, the “therapeutically effective amount” refers to the amount of an oligonucleotide decoy.

### Methods

The inventors have identified the score on the Pain Catastrophizing Scale as an unexpected predictor of response to brivoligide treatment. The present disclosure is based, in part, on an unexpected finding that patients with a high pain catastrophizing scale (PCS) score (e.g., a PCS score of 20 or greater or a PCS score of 16 or greater) show a clinically meaningful reduction in pain upon treatment with brivoligide, an oligonucleotide inhibitor of a transcription factor, EGR1. The findings presented in this disclosure are surprising and unexpected because prior to the present study, patients with high PCS scores were considered to be poorly-responsive to pain treatments. Accordingly, the present disclosure provides for the first time methods of treating or preventing pain in patients having high PCS scores.

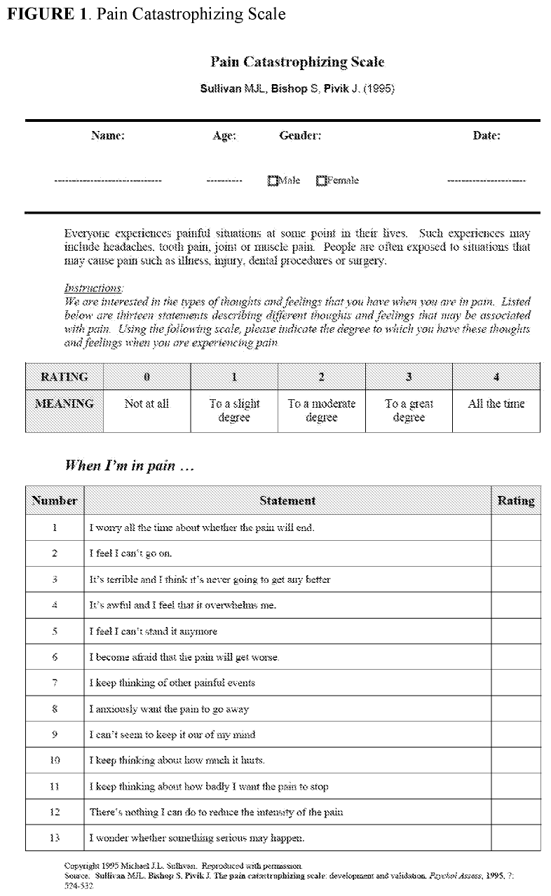

The Pain Catastrophizing Scale (PCS) was developed in 1995 and contains 13 items covering three components (rumination, helplessness and magnification). See FIG. 1 for the overview of the PCS. Each item is rated on a 5 point scale (0 to 4) and the total score ranges from 0 to 52.

The PCS has been translated and validated in Chinese, Japanese, French, German, Dutch, Spanish, Greek and Catalan. Validation includes: 1) Principal component analysis supports 3 components (helplessness, rumination and magnification), 2) Content validity—comparison of questionnaire response to interview based responses, 3) Construct validity—PCS compared with measures of related constructs including depression, trait anxiety, negative affectivity and fear of pain with little overlap; only PCS contributed significant unique variance to the prediction of pain intensity (Sullivan et al., “Theoretical perspectives on the relationship between catastrophizing and pain,” The Clinical Journal of Pain, 2001, 17:52-64), 4) Test-retest stability over 10 weeks, 5) Internal consistency—Chronbach's alpha ≥0.75->0.95, and 5) Clinical validity in over 100 studies demonstrating relationship between PCS and pain. Use of the PCS has been studied in postoperative, post trauma, chronic, acute and procedural pain, and inflammatory diseases.

High levels of catastrophizing are associated with a variety of poor outcomes, including but not limited to, enhanced neural responses to painful stimulation (Gracely R H, Brain 2004), increased postoperative pain intensity (Pinto, Pain 2012), increased analgesic use (Jacobsen P B, J Behav Med 1996), prescription opioid misuse (Martel, Drug Alcohol Depend 2013), increased frequency and duration of hospital stay (Gil K M, J Consult Clin Psychol 1992), more frequent visits to health care professionals (Gil K M, J Ped Psychol 1993), longer times to achieve postop functional targets (90° knee flexion) (Kendell K, Br J Health Psychol 2001), and onset of phantom limb pain after amputation (Richardson C, J Pain 2007). That is, until the present study, effective treatments to manage pain experienced by patients with high levels of PCS scores were not available. Treatment of patients with high PCS scores (e.g., a PCS score of 20 or greater or a PCS score of 16 or greater) using the methods of the present disclosure provides clinically meaningful improvement in one or more of these outcomes.

In some embodiments, methods for treating or preventing pain in a patient having a high PCS score comprise administering to said patient an oligonucleotide inhibitor of a transcription factor or a pharmaceutical composition comprising an oligonucleotide inhibitor of a transcription factor. In some embodiments, patients with a high PCS score that can be treated with the methods of the present disclosure have a PCS score of ≥20 or ≥16. The phrase “PCS score of ≥20 or ≥16” does not mean a “narrow range” of only 16 to 20 on the PCS scale; rather, the phrase means that in some aspects, a patient will have a PCS score of 16 or greater, and in some aspects the patient will have a PCS score of 20 or greater. Consequently, the 16 or 20 is demarcating the lower level baseline cutoff of PCS score. Further information on the PCS scores and that a score of at least 16 on the PCS is considered high can be found in the following references incorporated herein by reference: (a) Scott, W. et al, “Clinically Meaningful Scores on Pain Catastrophizing Before and After Multidisciplinary Rehabilitation,” Clin J Pain 2014; 30 (3):183-190, (b) Sullivan, Bishop, and Pivik, “The Pain Catastrophizing Scale: Development and Validation,” Psychological Assessment, 1995, Vol. 7, No. 4, 524-532, (c) Riddle, D L et al., “Preoperative Pain Catastrophizing Predicts Pain Outcome after Knee Arthroplasty,” Clin Orthop Relat Res, 2010, 468:798-806.

In some embodiments, provided herein are methods for treating post-surgical pain in patients scoring ≥20 or ≥16 on the PCS scale preoperatively, the method comprising administering to said patient brivoligide or a pharmaceutical composition comprising briovoligide. In some embodiments, said methods may not be effective in patients scoring less than 20 or less than 16 on the PCS scale.

In some embodiments, an oligonucleotide inhibitor of a transcription factor administered to patients with a high PCS score, for example, a PCS score of ≥20 or ≥16, is an oligonucleotide decoy comprising one or more transcription factor binding sites. In some embodiments, methods for treating or preventing pain in a patient having a high PCS score (e.g., PCS ≥20 or ≥16) comprise administering to said patient any one of the oligonucleotide decoys described herein. In exemplary embodiments, methods for treating or preventing pain in a patient having a high PCS score (e.g., PCS ≥20 or ≥16) comprise administering to said patient an oligonucleotide decoy comprising a sequence selected from SEQ ID NOs: 1-53.

In some embodiments, methods for treating or preventing pain in a patient having a high PCS score (e.g., PCS ≥20 or ≥16) comprise administering to said patient an oligonucleotide decoy comprising one or more transcription factor binding sites, wherein the one or more transcription factor binding sites bind to a transcription factor selected from the group consisting of: POU1F1, POU2F, POU3F, POU4F1, POU5F1, USF, EGR1, CREB/ATF, AN, CEBP, SRF, ETS1, MEF2, SP1, RUNX, NFAT, ELK1, ternary complex factors, STAT, GATA1, ELF1, nuclear factor—granulocyte/macrophage a, HNF1, ZFHX3, IRF, TEAD1, TBP, NFY, caccc-box binding factors, KLF4, KLF7, IKZF, MAF, REST, HSF, KCNIP3 and PPAR.

In an exemplary embodiment, methods for treating or preventing pain in a patient having a high PCS score (e.g., PCS ≥20 or ≥16) comprise administering to said patient an oligonucleotide decoy comprising one or more transcription factor binding sites for the transcription factor Early Growth Response protein 1 (EGR1).

In some embodiments, methods for treating or preventing pain in a patient having a high PCS score (e.g., PCS ≥20 or ≥16) comprise administering to said patient an oligonucleotide decoy comprising: (a) a sequence selected from the group consisting of SEQ ID NOs: 1-53; or (b) a sequence having at least 50%, 55%, 60%, 65%, 70%, 75%, 80%, 85%, 88%, 89%, 90%, 91%, 92%, 93%, 94%, 95%, 96%, 97%, 98%, 99%, or 100% identity with a sequence selected from the group consisting of SEQ ID NOs.: 1-53.

In some embodiments, methods for treating or preventing pain in a patient having a high PCS score (e.g., PCS ≥20 or ≥16) comprise administering to said patient an oligonucleotide decoy comprising: (a) a sequence selected from the group consisting of SEQ ID NOs.: 1-40, 42, 45 and 47-53; or (b) a sequence having at least 50%, 55%, 60%, 65%, 70%, 75%, 80%, 85%, 88%, 89%, 90%, 91%, 92%, 93%, 94%, 95%, 96%, 97%, 98%, 99%, or 100% identity with a sequence selected from the group consisting of SEQ ID NOs.: 1-40, 42, 45 and 47-53.

In some embodiments, methods for treating or preventing pain in a patient having a high PCS score (e.g., PCS ≥20 or ≥16) comprise administering to said patient an oligonucleotide decoy comprising a sense strand having a sequence selected from the group consisting of SEQ ID NOs: 1-53. In these embodiments, the oligonucleotide decoy may comprise an antisense strand that (a) has a sequence that is fully complementary to the sense strand sequence selected from SEQ ID NOs: 1-53 or (b) has a sequence that is at least 90% complementary to the sense strand sequence selected from SEQ ID NOs: 1-53.

Methods of the present disclosure can be used for treating or preventing pen-operative pain in a high PCS score patient (e.g., PCS ≥20 or ≥16). In one embodiment, methods of the present disclosure are used for treating or preventing post-operative pain, e.g., post-surgical pain, in a high PCS score patient (e.g., PCS ≥20 or ≥16).

In certain embodiments, an oligonucleotide inhibitor (e.g., an oligonucleotide decoy) and/or pharmaceutical composition thereof is administered to a high PCS score patient (e.g., PCS ≥20 or ≥16) suffering from pain including, but not limited to: mechanical pain (e.g., mechanical hyperalgesia and/or allodynia), chemical pain, temperature pain, chronic pain, sub-chronic pain, acute pain, sub-acute pain, inflammatory pain, neuropathic pain, muscular pain, skeletal pain, post-surgery pain, arthritis pain, and diabetes pain. Further, in certain embodiments, the oligonucleotide inhibitors and/or pharmaceutical compositions thereof are administered to a high PCS score patient (e.g., PCS ≥20 or ≥16) as a preventative measure against pain including, but not limited to: post-operative pain, chronic pain, inflammatory pain, neuropathic pain, muscular pain, and skeletal pain. In certain embodiments, the oligonucleotide inhibitors and/or pharmaceutical compositions thereof may be used for the prevention and/or treatment and/or amelioration of one facet of pain while concurrently treating another symptom of pain.

In some other embodiments, pain or pain related conditions include post-operative pain, chronic pain, inflammatory pain, neuropathic pain, muscular pain, and skeletal pain.

Treatment of the patients who score high on the PCS (e.g., PCS score of 20 or greater or PCS score of 16 or greater) using the methods described herein provide a clinically meaningful outcome across various pain-related clinical endpoints. For example, in some aspects, methods of the present disclosure provide a clinically meaningful reduction in pain. In some aspects, methods of the present disclosure provide a clinically meaningful reduction in movement-evoked pain (e.g., pain with walking) and/or pain at rest. In some aspects, methods of the present disclosure provide a clinically meaningful reduction in worst pain, as measured by a 11 point Numerical Rating Scale (NRS). In some aspects, methods of the present disclosure provide a clinically meaningful reduction in time to achieve a change in the NRS score of ≤3 for worst pain. In some aspects, methods of the present disclosure provide a clinically meaningful reduction in opioid consumption.

In some embodiments, treatment of patients who score high on the PCS (e.g., PCS score of ≥20 or ≥16) using the methods described herein may provide a statistically significant outcome across various pain-related clinical endpoints. For example, in some aspects, methods of the present disclosure may provide a statistically significant reduction in pain. In some aspects, methods of the present disclosure may provide a statistically significant reduction in movement-evoked pain (e.g., pain with walking) and/or pain at rest. In some aspects, methods of the present disclosure may provide a statistically significant reduction in worst pain, as measured by a 11 point Numerical Rating Scale (NRS). In some aspects, methods of the present disclosure may provide a statistically significant reduction in time to achieve a change in the NRS score of ≤3 for worst pain. In some aspects, methods of the present disclosure provide may provide a statistically significant reduction in opioid consumption.

Methods of the present disclosure provide a clinically meaningful reduction in pain to the high PCS score patient (e.g., PCS score of ≥20 or ≥16) administered with the oligonucleotide inhibitors described herein compared to a high PCS score patient not administered with the oligonucleotide inhibitor. The statistically significant or clinically meaningful reduction in pain provided by the present methods to the patients with high PCS scores (e.g., a PCS score of ≥20 or ≥16) is surprising and unexpected as patients with such high PCS scores have been known to show a poor response to current pain treatments and are associated with poor postoperative outcomes.

In various embodiments, the patient administered with the oligonucleotide inhibitors described herein experiences a clinically meaningful reduction in pain through at least day 28, 42, or 90 post-surgery. In some embodiments, said reduction in pain experienced by said patient is at least an additional 20%, 25%, 30%, 35%, 40%, 45%, 50%, 55%, 60%, 65%, 70%, 75%, or 80% reduction compared to a patient not administered the oligonucleotide inhibitor. In an exemplary embodiment, said reduction in pain experienced by said patient is at least an additional 20% reduction compared to a patient not administered the oligonucleotide inhibitor. In another exemplary embodiment, said reduction in pain experienced by said patient is at least an additional 30% reduction compared to a patient not administered the oligonucleotide inhibitor.

In various embodiments, the patient administered with the oligonucleotide inhibitors described herein experiences a clinically meaningful reduction in movement-evoked pain and/or pain at rest. In some embodiments, said reduction in movement-evoked pain and/or pain at rest is experienced by said patient through at least day 28, 42, or 90 post-surgery. In some embodiments, said reduction in movement-evoked pain and/or pain at rest is experienced by said patient from about day 7 post-surgery through at least day 28; from about day 7 post-surgery through at least day 42 post-surgery; or from about day 7 post-surgery through at least day 90 post-surgery. In some of these embodiments, said reduction in movement-evoked pain and/or pain at rest is at least an additional 20%, 25%, 30%, 35%, 40%, 45%, 50%, 55%, 60%, 65%, 70%, 75%, or 80% reduction compared to a patient not administered the oligonucleotide inhibitor. In an exemplary embodiment, said reduction in movement-evoked pain and/or pain at rest experienced by said patient is at least an additional 20% reduction compared to a patient not administered the oligonucleotide inhibitor. In another exemplary embodiment, said reduction in movement-evoked pain and/or pain at rest experienced by said patient is at least an additional 30% reduction compared to a patient not administered the oligonucleotide inhibitor.

In some embodiments, the patient administered with the oligonucleotide inhibitors described herein experiences a reduction in movement-evoked pain and/or pain at rest, wherein said reduction in pain is at least a 0.5 to 1 point reduction, at least a 0.5 to 2 point reduction, at least a 0.5 to 3 point reduction, at least a 1 to 2 point reduction, at least a 2 to 3 point reduction, as measured by an 11 point numerical rating scale (NRS), compared to a patient not administered the oligonucleotide inhibitor. In some of these embodiments, said point reduction in movement-evoked pain and/or pain at rest as measured by the 11 point NRS is experienced by said patient through at least day 28, 42, or 90 post-surgery. In some embodiments, said point reduction in movement-evoked pain and/or pain at rest as measured by the 11 point NRS is experienced by said patient from about day 7 post-surgery through at least day 28; from about day 7 post-surgery through at least day 42 post-surgery; or from about day 7 post-surgery through at least day 90 post-surgery.

In some embodiments, the patient administered with the oligonucleotide inhibitors described herein experiences a reduction in movement-evoked pain and/or pain at rest that is at least an additional 20%, 25%, 30%, 35%, 40%, 45%, 50%, 55%, 60%, 65%, 70%, 75%, or 80% reduction, as measured by the 11 point NRS, compared to a patient not administered the oligonucleotide inhibitor. In some of these embodiments, said percent reduction in movement-evoked pain and/or pain at rest as measured by the 11 point NRS is experienced by said patient through at least day 28, 42, or 90 post-surgery. In some embodiments, said percent reduction in movement-evoked pain and/or pain at rest as measured by the 11 point NRS is experienced by said patient from about day 7 post-surgery through at least day 28; from about day 7 post-surgery through at least day 42 post-surgery; or from about day 7 post-surgery through at least day 90 post-surgery. In an exemplary embodiment, said reduction in movement-evoked pain and/or pain at rest experienced by said patient is at least an additional 20% reduction, as measured by the 11 point NRS, compared to a patient not administered the oligonucleotide inhibitor. In another exemplary embodiment, said reduction in movement-evoked pain and/or pain at rest experienced by said patient is at least an additional 30% reduction, as measured by the 11 point NRS, compared to a patient not administered the oligonucleotide inhibitor.

In some embodiments, time taken to achieve any of the above-described reduction in pain by the patient administered with the oligonucleotide inhibitors described herein is about 10 to 30 days less, about 10 to 25 days less, about 15 to 30 days less, about 15 to 25 days less, or about 20 to 26 days less compared to a patient not administered the oligonucleotide inhibitor.

Patients with high PCS scores (e.g., PCS score of ≥20 or ≥16) are known to be associated with increased and prolonged opioid use and therefore to be at increased risk of opioid abuse/misuse. In some embodiments, the present methods of treating or preventing pain provide a reduction in opioid consumption, and thereby a reduction in opioid abuse/misuse potential, by the high PCS score patient population. In some of these embodiments, opioid consumption by the patient administered with the oligonucleotide inhibitors described herein is reduced by about 5%, 10%, 15%, 20%, 25%, 30%, 35%, 40%, 45%, or 50%, compared to a patient not administered the oligonucleotide inhibitor.

In some embodiments, opioid consumption by the patient administered with the oligonucleotide inhibitors of the present disclosure is reduced compared to a patient not administered the oligonucleotide inhibitor. In some embodiments, opioid consumption by the patient administered with the oligonucleotide inhibitors is reduced from day 0 post-surgery through at least day 90 post-surgery compared to a patient not administered the oligonucleotide inhibitor. In some embodiments, opioid consumption by the patient administered with the oligonucleotide inhibitors described herein is reduced by about 5%, 10%, 15%, 20%, 25%, 30%, 35%, 40%, 45%, or 50%, compared to a patient not administered the oligonucleotide inhibitor.

In some embodiments, daily average opioid consumption by the patient administered with the oligonucleotide inhibitors as described herein is reduced compared to a patient not administered the oligonucleotide inhibitor. In some embodiments, daily average opioid consumption by the patient administered with the oligonucleotide inhibitors as described herein is reduced by about 5%, 10%, 15%, 20%, 25%, 30%, 35%, 40%, 45%, or 50%, compared to a patient not administered the oligonucleotide inhibitor.

Methods of the present disclosure have great clinical importance on several levels. The use of the PCS score allows preoperative identification of a brivoligide-responsive population. This population is associated with poor postoperative outcomes including treatment resistance, high pain intensity, longer rehabilitation and high analgesic use and opioid misuse. This population represents from 25 to 40% or more of the population depending upon the clinical setting. Overall this profile is unique among pain therapeutics and addresses an important medically underserved population.

### Routes of Administration and Dosage

The present methods for treatment or prevention of pain require administration of an oligonucleotide inhibitor (e.g., oligonucleotide decoy), or pharmaceutical composition thereof, to a patient who scores high on the PCS in need of such treatment or prevention. The compounds and/or pharmaceutical compositions thereof may be administered by any convenient route, for example, by infusion or bolus injection, by absorption through epithelial or mucocutaneous linings (e.g., oral mucosa, rectal and intestinal mucosa, etc.), or orally. Administration can be systemic or local. Various delivery systems are known, including, e.g., encapsulation in liposomes, microparticles, microcapsules, capsules, etc., that can be used to administer a compound and/or pharmaceutical composition thereof.

Methods of administration include, but are not limited to, intradermal, intramuscular, intraperitoneal, intravenous, subcutaneous, intranasal, epidural/peridural, intrathecal, oral, sublingual, intranasal, intracerebral, intravaginal, transdermal, rectally, by inhalation or topically, particularly to the ears, nose, eyes, or skin. In certain embodiments, more than one oligonucleotide inhibitor is administered to a patient. The mode of administration will depend in-part upon the site of the medical condition.

In specific embodiments, it may be desirable to administer one or more oligonucleotide inhibitors (e.g., oligonucleotide decoys) locally to the area in need of treatment. This may be achieved, for example, and not by way of limitation, by local infusion during surgery, topical application (e.g., in conjunction with a wound dressing after surgery), by injection, by means of a catheter, by means of a suppository, or by means of an implant, said implant being of a porous, non-porous, or gelatinous material, including membranes, such as sialastic membranes, or fibers. In some embodiments, administration can be by direct injection at the site (e.g., former, current, or expected site) of pain.

In certain embodiments, it may be desirable to introduce one or more oligonucleotide inhibitors (e.g., oligonucleotide decoys) into the nervous system by any suitable route, including but not restricted to intraventricular, intrathecal, perineural and/or epidural/peridural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant.

The amount of oligonucleotide inhibitor that will be effective in the treatment or prevention of pain in a patient will depend on the specific nature of the condition and can be determined by standard clinical techniques known in the art. In addition, in vitro or in vivo assays may optionally be employed to help identify optimal dosage ranges. The amount of an oligonucleotide inhibitor (e.g., oligonucleotide decoy) administered will, of course, be dependent on, among other factors, the subject being treated, the weight of the subject, the severity of the affliction, the manner of administration, and the judgment of the prescribing physician. In certain embodiments, a single dose of an oligonucleotide inhibitor (e.g., oligonucleotide decoy) comprises about 1 mg to about 3000 mg, 1 mg to about 2000 mg, 1 mg to about 1500 mg, 1 mg to about 1200 mg, 1 mg to about 1100 mg, 100 mg to about 3000 mg, 100 mg to about 2000 mg, 100 mg to about 1500 mg, 100 mg to about 1200 mg, 100 mg to about 1100 mg, 200 mg to about 3000 mg, 200 mg to about 2000 mg, 200 mg to about 1500 mg, 200 mg to about 1200 mg, 200 mg to about 1100 mg, 300 mg to about 3000 mg, 300 mg to about 2000 mg, 300 mg to about 1500 mg, 300 mg to about 1200 mg, 300 mg to about 1100 mg, 400 mg to about 3000 mg, 400 mg to about 2000 mg, 400 mg to about 1500 mg, 400 mg to about 1200 mg, 400 mg to about 1100 mg, 500 mg to about 3000 mg, 500 mg to about 2000 mg, 500 mg to about 1500 mg, 500 mg to about 1200 mg, 500 mg to about 1100 mg, 600 mg to about 3000 mg, 600 mg to about 2000 mg, 600 mg to about 1500 mg, 600 mg to about 1200 mg, 600 mg to about 1100 mg, 700 mg to about 3000 mg, 700 mg to about 2000 mg, 700 mg to about 1500 mg, 700 mg to about 1200 mg, 700 mg to about 1100 mg, 800 mg to about 3000 mg, 800 mg to about 2000 mg, 800 mg to about 1500 mg, 800 mg to about 1200 mg, 800 mg to about 1100 mg, 900 mg to about 3000 mg, 900 mg to about 2000 mg, 900 mg to about 1500 mg, 900 mg to about 1200 mg, 900 mg to about 1100 mg, of the oligonucleotide inhibitor per patient. Further, one embodiment may comprise administering 1100 mg±500 mg of an oligonucleotide inhibitor (e.g. oligonucleotide decoy) per patient. Another embodiment may comprise administering 1100 mg±400 mg of an oligonucleotide inhibitor (e.g. oligonucleotide decoy) per patient. Another embodiment may comprise administering 1100 mg±300 mg of an oligonucleotide inhibitor (e.g. oligonucleotide decoy) per patient. Another embodiment may comprise administering 1100 mg±200 mg of an oligonucleotide inhibitor (e.g. oligonucleotide decoy) per patient. Another embodiment may comprise administering 1100 mg±100 mg of an oligonucleotide inhibitor (e.g. oligonucleotide decoy) per patient. Another embodiment may comprise administering 1100 mg±50 mg of an oligonucleotide inhibitor (e.g. oligonucleotide decoy) per patient. Another embodiment may comprise administering 1100 mg±10 mg of an oligonucleotide inhibitor (e.g. oligonucleotide decoy) per patient. Another embodiment may comprise administering 1100 mg±50% of an oligonucleotide inhibitor (e.g. oligonucleotide decoy) per patient. Another embodiment may comprise administering 1100 mg±40% of an oligonucleotide inhibitor (e.g. oligonucleotide decoy) per patient. Another embodiment may comprise administering 1100 mg±30% of an oligonucleotide inhibitor (e.g. oligonucleotide decoy) per patient. Another embodiment may comprise administering 1100 mg±20% of an oligonucleotide inhibitor (e.g. oligonucleotide decoy) per patient. Another embodiment may comprise administering 1100 mg±10% of an oligonucleotide inhibitor (e.g. oligonucleotide decoy) per patient. Another embodiment may comprise administering 1100 mg±5% of an oligonucleotide inhibitor (e.g. oligonucleotide decoy) per patient.

In certain embodiments, a single dose of an oligonucleotide inhibitor (e.g. oligonucleotide decoy) comprises about: 100 mg to about 700 mg, 150 mg to about 700 mg, 200 mg to about 700 mg, 250 mg to about 700 mg, 300 mg to about 700 mg, 350 mg to about 700 mg, 400 mg to about 700 mg, 450 mg to about 700 mg, 500 mg to about 700 mg, 550 mg to about 700 mg, 600 mg to about 700 mg, or 650 mg to about 700 mg. Further, one embodiment may comprise administering 660 mg±330 mg of an oligonucleotide inhibitor (e.g. oligonucleotide decoy) per patient. Another embodiment may comprise administering 660 mg±260 mg of an oligonucleotide inhibitor (e.g. oligonucleotide decoy) per patient. Another embodiment may comprise administering 660 mg±200 mg of an oligonucleotide inhibitor (e.g. oligonucleotide decoy) per patient. Another embodiment may comprise administering 660 mg±130 mg of an oligonucleotide inhibitor (e.g. oligonucleotide decoy) per patient. Another embodiment may comprise administering 660 mg±60 mg of an oligonucleotide inhibitor (e.g. oligonucleotide decoy) per patient. Another embodiment may comprise administering 660 mg±30 mg of an oligonucleotide inhibitor (e.g. oligonucleotide decoy) per patient. Another embodiment may comprise administering 660 mg±10 mg of an oligonucleotide inhibitor (e.g. oligonucleotide decoy) per patient. Another embodiment may comprise administering 660 mg±50% of an oligonucleotide inhibitor (e.g. oligonucleotide decoy) per patient. Another embodiment may comprise administering 660 mg±40% of an oligonucleotide inhibitor (e.g. oligonucleotide decoy) per patient. Another embodiment may comprise administering 660 mg±30% of an oligonucleotide inhibitor (e.g. oligonucleotide decoy) per patient. Another embodiment may comprise administering 660 mg±20% of an oligonucleotide inhibitor (e.g. oligonucleotide decoy) per patient. Another embodiment may comprise administering 660 mg±10% of an oligonucleotide inhibitor (e.g. oligonucleotide decoy) per patient. Another embodiment may comprise administering 660 mg±5% of an oligonucleotide inhibitor (e.g. oligonucleotide decoy) per patient. Another embodiment may comprise administering 660 mg±1% of an oligonucleotide inhibitor (e.g. oligonucleotide decoy) per patient.

In aspects, the dosage forms may be administered to a patient once per day. Dosing may be provided alone or in combination with other drugs and may continue as long as required for effective treatment or prevention of pain.

### Pharmaceutical Compositions

The pharmaceutical compositions disclosed herein comprise a therapeutically effective amount of one or more oligonucleotide inhibitors (e.g. one or more oligonucleotide decoys), preferably, in purified form, together with a suitable amount of a pharmaceutically acceptable vehicle, so as to provide a form for proper administration to a patient. When administered to a patient, oligonucleotide inhibitors and pharmaceutically acceptable vehicles are preferably sterile. Water can be a vehicle when oligonucleotide inhibitors are administered intravenously. Saline solutions and aqueous dextrose and glycerol solutions can also be employed as liquid vehicles, particularly for injectable solutions. Suitable pharmaceutical vehicles include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compounds disclosed herein into preparations which can be used pharmaceutically. Proper formulation is dependent upon the route of administration chosen.

The present pharmaceutical compositions can take the form of solutions, suspensions, emulsions, tablets, pills, pellets, capsules, capsules containing liquids, powders, sustained-release formulations, suppositories, aerosols, sprays, suspensions, or any other form suitable for use. Other examples of suitable pharmaceutical vehicles have been described in the art (see Remington's Pharmaceutical Sciences, Philadelphia College of Pharmacy and Science, 19th Edition, 1995).

Pharmaceutical compositions for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame or saccharin, flavoring agents such as peppermint, oil of wintergreen, or cherry coloring agents and preserving agents, to provide a pharmaceutically palatable preparation. Moreover, when in tablet or pill form, the compositions may be coated to delay disintegration and absorption in the gastrointestinal tract, thereby providing a sustained action over an extended period of time. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol), oils, alcohols, slightly acidic buffers between pH 4 and pH 6 (e.g., acetate, citrate, or ascorbate at between about 5 mM to about 50 mM), etc. Additionally, flavoring agents, preservatives, coloring agents, bile salts, acylcarnitines and the like may be added.

Compositions for administration via other routes may also be contemplated. For buccal administration, the compositions may take the form of tablets, lozenges, etc., formulated in conventional manner. Liquid drug formulations suitable for use with nebulizers and liquid spray devices and EHD aerosol devices will typically include a compound with a pharmaceutically acceptable vehicle. The pharmaceutically acceptable vehicle may be a liquid such as alcohol, water, polyethylene glycol or a perfluorocarbon. Optionally, another material may be added to alter the aerosol properties of the solution or suspension of compounds. This material may be liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611).

A compound may be formulated for intrathecal injection.

A compound may be formulated for delivery using ultrasound-release methods.

A compound may also be formulated in rectal or vaginal compositions such as suppositories or retention enemas, e.g., containing conventional suppository bases such as cocoa butter or other glycerides.

In addition to the formulations described previously, a compound may also be formulated as a depot preparation. Such long acting formulations may be administered by implantation (for example, subcutaneously or intramuscularly) or by intramuscular injection. Thus, for example, a compound may be formulated with suitable polymeric or hydrophobic materials (for example, as an emulsion in an acceptable oil) or ion exchange resins, or as sparingly soluble derivatives, for example, as a sparingly soluble salt.

An oligonucleotide inhibitor (e.g., an oligonucleotide decoy) may be included in any of the above-described formulations, or in any other suitable formulation, as a pharmaceutically acceptable salt, a solvate or hydrate. Pharmaceutically acceptable salts substantially retain the activity of the parent compound and may be prepared by reaction with appropriate bases or acids and tend to be more soluble in aqueous and other protic solvents than the corresponding parent form.

According to the present invention, the composition of the present invention can further comprise a buffer. Any suitable buffer can be used for the composition of the present invention. In some other embodiments, the buffer system used for the composition of the present invention is an organic or inorganic buffer. Examples of buffers include phosphate buffers, citrate buffers, borate buffers, bicarbonate buffers, carbonate buffers, acetate buffers, ammonium buffers, and tromethamine (Tris) buffers.

According to the present invention, in some embodiments, when the active ingredient is an oligonucleotide and the agent is an ion, e.g., calcium, the buffer is a non-phosphate based buffer. The amount of buffer employed will be ascertainable to a skilled artisan, such as an amount ranging from 0.01 mM to 1 M, such as 10 mM.

Intrathecal administration is a route of administration to deliver drugs through the spinal sac and directly into the cerebrospinal fluid (CSF).

### Combination Therapy

In certain embodiments, oligonucleotide inhibitors (e.g., oligonucleotide decoys) and/or pharmaceutical compositions thereof can be used in combination therapy with at least one other therapeutic agent, which may include, but is not limited to, an oligonucleotide inhibitor. The oligonucleotide inhibitors and/or pharmaceutical composition thereof and the therapeutic agent can act additively or, more preferably, synergistically. In some embodiments, an oligonucleotide inhibitors and/or a pharmaceutical composition thereof is administered concurrently with the administration of another therapeutic agent, including another oligonucleotide inhibitor. In other embodiments, an oligonucleotide inhibitor or a pharmaceutical composition thereof is administered prior or subsequent to administration of another therapeutic agent, including another oligonucleotide inhibitor.

### Formulations of an Oligonucleotide Inhibitor and a Stabilizing Agent

In one aspect, methods of the present invention comprise administering a composition, such as a pharmaceutical composition, comprising an active ingredient and an agent associated, directly or indirectly, with one or more adverse effect(s) of the active ingredient. In one embodiment, the agent is any entity, the homeostatic levels of which are directly or indirectly related to one or more adverse effect(s) of the active ingredient. In another embodiment, the agent is any entity, the homeostatic levels of which are changed, e.g., substantially upon administration of the active ingredient in vivo. In yet another embodiment, the agent is any entity, the homeostatic levels of which are sensitive to the administration of the active ingredient in vivo. In still another embodiment, the agent is any entity which is capable of interacting or interacts, directly or indirectly, with the active ingredient. In still yet another embodiment, the agent is any entity which is capable of binding or binds, directly or indirectly, with the active ingredient.

In some embodiments, the agent can be different, e.g., even with respect to the same active ingredient, depending on the tissue or cell type the active ingredient is administered into. In some embodiments, the agent is an ion. An ion can be an organic acid, such as malic, ascorbic, tartaric, lactic, acetic, formic, oxalic, or citric acid. In some embodiments, the agent is a metal ion, e.g., iron, zinc, copper, lead and nickel, etc. In some embodiments, the agent has a charge that is opposite of the net charge of the active ingredient. In some embodiments, the agent is a cation or anion. In some other embodiments, the agent is a calcium ion, a magnesium ion, or a potassium ion. In some other embodiments, the agent is an ion, carbohydrate (e.g., sugars, starches, etc.), lipid (e.g., saturated fatty acids, unsaturated fatty acids, triacylglycerols, glycerophospholipids, sphingolipids, and cholesterol, etc.), vitamin (e.g., selenium, zinc, vitamin A, thiamine, riboflavin, pyridoxin, niacin, pantothenic acid, cyanocobalamin, L-ascorbic acid and □-tocopherol, etc.), or alcohol (e.g., polyols such as glucose and mannitol, as well as, e.g., ethanol, etc.) or a combination thereof.

In yet further embodiments, the agent with respect to cerebrospinal fluid is an ion, e.g., calcium ions, magnesium ions or potassium ions.

In still some other embodiments, the agent with respect to blood is one or more blood electrolytes and/or major constituents of extracellular, cellular and interstitial fluids. In some exemplary embodiments, the agent with respect to blood is Na⁺, K⁺, Ca²⁺, Mg²⁺, Cl⁻, bicarbonates (e.g., HCO₃⁻), phosphorus (e.g., HPO₄²⁻), sulfates (e.g., SO₄²⁻), organic acid, proteins, metal ions (iron, zinc, copper, lead and nickel, etc.), carbohydrates or alcohols (e.g., glucose, mannitol, ethanol), lipids, vitamins (e.g., selenium, zinc) or any combination thereof.

According to the present invention, the agent used in the composition of the active ingredient can be any amount suitable for the administration of the active ingredient in vivo, e.g., any amount that either inhibits or decreases one or more adverse effect(s) of the active ingredient without the agent.

According to the present invention, one or more adverse effect(s) of the active ingredient includes any unwanted or undesirable effect produced as a result of an in vivo administration of the active ingredient. An adverse effect can be any long term or short effect, local or systematic effect, or any effect associated with the toxicity of the active ingredient. Exemplary adverse effects include pain, headache, vomiting, arrhythmia, shivering, respiratory depression, dizziness, loss of motor control, lack of coordination, fatigue, memory impairment, rash, or numbness. In one embodiment, the adverse effect in the context of pain treatment with an oligonucleotide decoy can be relatively minor (e.g., light tail movement in a rodent or dog animal model) or more severe (e.g., a seizure), or may include muscle trembling, increased muscle tone in a limb, whole body rigidity, pain, or spontaneous vocalization.

In one embodiment, the agent used in the composition of the active ingredient is an in vivo stabilizing amount. As used herein, an “in vivo stabilizing amount” is an amount of the agent that upon administration along with the active ingredient does not cause any material or detectable change of the endogenous level, e.g., homeostatic level of the agent in vivo. Alternatively an “in vivo stabilizing amount” is an amount of the agent that upon administration along with the active ingredient inhibits or decreases one or more adverse effect(s) of the active ingredient without the agent.

In some embodiments, the in vivo stabilizing amount of the agent is an amount that sufficiently saturates binding sites, e.g., available binding sites of the active ingredient to the agent. For example, the in vivo stabilizing amount of the agent can be an amount that capable of binding or binds to at least 0.001%, 0.1%, 0.5%, 1%, 2%, 5%, 10%, 20%, 30%, 40%, or 50% of binding sites, e.g., available binding sites of the active ingredient to the agent. In some other embodiments, the in vivo stabilizing amount of the agent is an amount that upon administration along with the active ingredient does not materially affect or cause detectable change of the pH (e.g., induces a change less than about 0.5 pH units, 0.2 pH units, 0.1 pH units, etc.) of the local site, tissue, or cell environment, etc.

In yet some other embodiments, the in vivo stabilizing amount of the agent is the amount that upon mixing with the active ingredient produces less than a predetermined level of free agent in the composition, e.g., minimum or undetectable level of free agent in the composition. For example, the predetermined level of free agent in the composition can be at least less than 0.1 mM, 0.5 mM, 1 mM, 1.5 mM, or 2 mM in a composition when the active ingredient is an oligonucleotide decoy and the agent is an ion, e.g., calcium. In another example, the predetermined level of the free agent in the composition is less than about 1%, 2%, 5%, 10%, 20%, 30%, 40%, 50%, 60%, 70%, 80%, 90% or 100% of the endogenous level, e.g., local concentration of the agent. In yet another example, the predetermined level of free agent in the composition is determined based on the saturation level of the binding sites in the active ingredient to the agent.

According to the present invention, the free agent is the agent that is not bound to the active ingredient, e.g., by electrostatic, covalent, or hydrophobic interactions, or any other mode of interaction. Alternatively the free agent is the agent that is capable of interfering or interferes with the endogenous level of the agent, e.g., systematically or at the local site of administration.

In still some other embodiments, the in vivo stabilizing amount of the agent is the amount that provide suitable ratio between the active ingredient and the agent so that when they are administered in vivo, it inhibits or decreases one or more adverse effect(s) of the active ingredient without the agent or alternatively it does not cause substantial or detectable change of endogenous level, e.g., homeostatic level of the agent. In some embodiments, the molar ratio or the weight ratio of the active ingredient to the agent ranges from about 1:1000 to about 1000:1. Non-limiting examples of ratios include 1:1, 1:5, 1:10, 1:50, 1:100, 1:250, 1:500, 1:1000, 1000:1, 500:1, 250:1, 100:1, 50:1, 10:1, 5:1, and any range derivable therein inclusive of fractions of integers (e.g., 100.5, 100.05, etc.). Further non-limiting examples of ratios include 1:1, 1:2, 1:3, 1:4, 1:5, 1:6, 1:7, 1:8, 1:9, 1:10, 10:1, 9:1, 8:1, 7:1, 6:1, 5:1, 4:1, 3:1, and 2:1, and any range derivable therein, inclusive of fractions of integers (e.g., 1.5, 1.05, etc.).

In some embodiments, the active ingredient is a nucleic acid, such as an oligonucleotide (e.g., an oligonucleotide decoy), and the agent is a calcium ion, and wherein the weight ratio or the molar ratio of the active ingredient and the agent is from about 0.005 to 5, 0.05 to 5, 0.1 to 3, 0.2 to 2.8, 0.5 to 2, or 1 to 2.

In some embodiments, the active ingredient is a nucleic acid, such as an oligonucleotide (e.g., an oligonucleotide decoy), and the agent is a calcium ion, and wherein the weight ratio or the molar ratio of the active ingredient and the agent is from about 1 to 0.001, 1 to 0.005, 1 to 0.01, 1 to 0.015, 1 to 0.018, 1 to 0.019, 1 to 0.02, 1 to 0.025, 1 to 0.03, 1 to 0.035, 1 to 0.4, or 1 to 0.5. For example, the weight ratio may be 1:1, 2:1, 4:1, 5:1, 15:1, 30:1, 50:1, 100:1, 200:1, 250:1, 300:1, 400:1, 500:1, or 1000:1.

An agent, such as an ion (e.g., a calcium ion), can be comprised in a composition such as a salt (e.g., CaCl₂)), and the molar amount or weight amount of that composition can be referenced in a ratio. Accordingly, in some embodiments, the agent is a calcium ion comprised in a composition such as CaCl₂), wherein the weight ratio of an active ingredient, such as a nucleic acid (e.g., an oligonucleotide, an oligonucleotide decoy) to the composition, e.g., CaCl₂), is about 1:1, 2:1, 4:1, 5:1, 15:1, 30:1, 50:1, 100:1, 200:1, 250:1, 300:1, 400:1, or 500:1, or any range derivable therein.

It is understood that the exact ratio of active ingredient to agent in a composition may vary, such as based on the chemical nature of the active ingredient (e.g., in the context of a nucleic acid, whether the nucleic acid is RNA, DNA, single stranded or double stranded, the percent GC content, or molecular weight), the agent and its local concentration (e.g., endogenous level) in the targeted in vivo site, and its intended delivery route. For example, in an environment with a higher endogenous calcium concentration, it is anticipated that the ratio of active ingredient (e.g., oligonucleotide decoy):calcium should be increased in a composition comprising such components.

In still yet some other embodiments, the in vivo stabilizing amount of the agent is the amount that when administered along with the active ingredient causes minimum, insubstantial, or undetectable amount of interaction, e.g., binding between the endogenous agent and the active ingredient. In some aspects, the formulations present in U.S. Pat. No. 9,700,624 (incorporated herein by reference) are utilized herein. For example, in some embodiments, methods of treating or preventing pain according to the present disclosure comprise administering to a patient having a high PCS score a pharmaceutical composition formulated for administration to cerebrospinal fluid, comprising a) an oligonucleotide decoy having one or more EGR1 transcription factor binding sites; and b) an in vivo stabilizing amount of a calcium ion, wherein the oligonucleotide decoy is associated with neuromuscular adverse effects in vivo caused by the administration of the oligonucleotide decoy to cerebrospinal fluid without the calcium ion, said adverse effects resulting from the oligonucleotide decoy substantially binding endogenous calcium ion present in the cerebrospinal fluid, and wherein the in vivo stabilizing amount is the amount that substantially saturates the binding sites of the oligonucleotide decoy to the calcium ion thereby preventing the oligonucleotide decoy from substantially binding endogenous calcium ion present in the cerebrospinal fluid. In some embodiments, an oligonucleotide decoy having one or more EGR1 transcription factor binding sites comprises the sequence of SEQ ID NO: 42.

### Oligonucleotide Inhibitors

Methods of the present disclosure comprise administering an oligonucleotide inhibitor of a transcription factor to a patient with a high PCS score (≥20 or ≥16) for the treatment or prevention of pain. An oligonucleotide inhibitor of a transcription factor can be a single-stranded or double-stranded nucleic acid containing polymer. The oligonucleotide inhibitors used in the present methods may comprise DNA nucleotides, RNA nucleotides, modified nucleotides such as nucleotides containing sugar, base, and/or backbone modifications, conjugation to other molecules or a combination thereof.

In some embodiments, oligonucleotide inhibitors used in the methods of the present disclosure include oligonucleotide decoys. For example, an oligonucleotide decoy, such as described in U.S. Pat. Nos. 7,943,591; 8,093,225; 8,741,864, and U.S. application Ser. Nos. 14/258,927 and 15/019,791. An “oligonucleotide decoy” refers to any double-stranded, nucleic acid-containing polymer generally less than approximately 200 nucleotides (or 100 base pairs) and including, but not limited to, DNA, RNA and RNA-DNA hybrids. The term encompasses sequences that include any of the known base analogs of DNA and RNA including, but not limited to, 2,6-diaminopurine, 5-carboxymethylaminomethyl-2-thiouracil, 5-carboxymethylaminomethyluracil, dihydrouracil, inosine, uracil-5-oxyacetic acid, N6-isopentenyladenine, 1-methyladenine, N-uracil-5-oxyacetic acid methylester, queosine, 2-thiocytosine, 5-bromouracil, methylphosphonate, phosphorodithioate, ormacetal, 3′-thioformacetal, nitroxide backbone, sulfone, sulfamate, morpholino derivatives, locked nucleic acid (LNA) derivatives, or peptide nucleic acid (PNA) derivatives. In some embodiments, the oligonucleotide decoy is composed of two complementary single-stranded oligonucleotides that are annealed together. In other embodiments, the oligonucleotide decoy is composed of one single-stranded oligonucleotide that forms intramolecular base pairs to create a substantially double-stranded structure.

In certain embodiments, the oligonucleotide decoys comprise one or more (e.g., 1, 2, 3, 4, 5, etc.) transcription factor binding sites. In related embodiments, each transcription factor binding site binds to a transcription factor selected from the group consisting of POU1F1, POU2F, POU3F, POU4F1, POU5F1, USF, EGR1, CREB/ATF, AP1, CEBP, SRF, ETS1, MEF2, SP1, RUNX, NFAT, ELK1, ternary complex factors, STAT, GATA1, ELF1, nuclear factor—granulocyte/macrophage a, HNF1, ZFHX3, IRF, TEAD1, TBP, NFY, caccc-box binding factors, KLF4, KLF7, IKZF, MAF, REST, HSF, KCNIP3 and PPAR transcription factors. In certain embodiments, transcription factor binding sites bind to two or more members of a family of closely-related transcription factors. Representative members of such transcription factor families can be selected from the group consisting of POU1F1, POU2F, POU3F, POU4F1, POU5F1, USF, EGR1, CREB/ATF, AP1, CEBP, SRF, ETS1, MEF2, SP1, RUNX, NFAT, ELK1, ternary complex factors, STAT, GATA1, ELF1, nuclear factor—granulocyte/macrophage a, HNF1, ZFHX3, IRF, TEAD1, TBP, NFY, caccc-box binding factors, KLF4, KLF7, IKZF, MAF, REST, HSF, KCNIP3 and PPAR transcription factors. Thus, in certain embodiments, an oligonucleotide decoy that binds to, e.g., EGR1, can also bind to one or more additional family members, e.g., EGR2, EGR3, EGR4.

In certain embodiments, the oligonucleotide decoys comprise two or more (e.g., 2, 3, 4, 5, etc.) transcription factor binding sites. In related embodiments, each transcription factor binding site binds to a transcription factor selected from the group consisting of POU1F1, POU2F, POU3F, POU4F1, POU5F1, USF, EGR1, CREB/ATF, AP1, CEBP, SRF, ETS1, MEF2, SP1, RUNX, NFAT, ELK1, ternary complex factors, STAT, GATA1, ELF1, nuclear factor—granulocyte/macrophage a, HNF1, ZFHX3, IRF, TEAD1, TBP, NFY, caccc-box binding factors, KLF4, KLF7, IKZF, MAF, REST, HSF, KCNIP3 and PPAR transcription factors. In certain embodiments, the relative position of the two or more transcription factor binding sites within the decoy modulates (e.g., increases or decreases) the binding affinity between a target transcription factor (i.e., the transcription factor that a particular binding site is designed to bind to) and its transcription factor binding site, e.g., as compared to the binding affinity between the transcription factor and a decoy having a single transcription factor binding site (e.g., a consensus binding site) specific to the transcription factor. Thus, the relative position of the two transcription factor binding sites within an oligonucleotide decoy of the invention can increase the affinity of the oligonucleotide decoy for a target transcription factor (e.g., for one or more of the transcription factors targeted by the decoy). In certain embodiments, the increase in affinity of the oligonucleotide decoy for a target transcription factor is 1.2 fold or greater (e.g., about 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 fold, or more). In certain embodiments, the relative position of the two transcription factor binding sites within an oligonucleotide decoy promotes protein-protein interactions between transcription factors bound to the sites, e.g., homodimerization or heterodimerization of the transcription factors. In certain embodiments, such protein-protein interactions between transcription factors stabilize their interactions, e.g., binding, to the oligonucleotide decoy, thereby increasing the binding affinity of the oligonucleotide decoy for one or more of the target transcription factors.

In certain embodiments, a transcription factor that binds to a transcription factor binding site present in an oligonucleotide decoy is a human transcription factor. In other embodiments, the transcription factor that binds to a transcription factor binding site in an oligonucleotide decoy is a non-human, e.g., an avian, mammal (e.g., mouse, rat, dog, cat, horse, cow, etc.), or primate, transcription factor.

In certain embodiments, the transcription factor binding sites of an oligonucleotide decoy each bind to the same transcription factor, e.g., EGR1. In other embodiments, the transcription factor binding sites of an oligonucleotide decoy bind to different transcription factors, e.g., different members of a closely related family of transcription factors (e.g., different members of the EGR1 family) or a combination of transcription factors selected from the group consisting of POU1F1, POU2F, POU3F, POU4F1, POU5F1, USF, EGR1, CREB/ATF, AP1, CEBP, SRF, ETS1, MEF2, SP1, RUNX, NFAT, ELK1, ternary complex factors, STAT, GATA1, ELF1, nuclear factor—granulocyte/macrophage a, HNF1, ZFHX3, IRF, TEAD1, TBP, NFY, caccc-box binding factors, KLF4, KLF7, IKZF, MAF, REST, HSF, KCNIP3 and PPAR transcription factors.

In certain embodiments, the transcription factor binding sites of an oligonucleotide decoy are separated from each other by a linker sequence. Linker sequences can be 1, 2, 3, 4, 5, 6, 7, 8, 9, 10, or more base pairs in length. Typically, linker sequences will be two to five base pairs in length. In other embodiments, the transcription factor binding sites can be immediately adjacent to one another (e.g., no linker sequence is present) or overlapping. In cases where the transcription factor binding sites are overlapping, the transcription factor binding sites can share 1, 2, 3, 4, 5, or more base pairs. Alternatively, one or both of the transcription factor binding sites can be lacking base pairs that otherwise form part of a consensus binding sequence for the transcription factor(s) that bind to the site. In general, however, base pairs that are critical to the binding interaction between a transcription factor binding site and the transcription factors that bind to the site (e.g., base pairs that are essentially invariant in a consensus binding sequence for a particular transcription factor) are not shared or missing when transcription binding sequences are overlapping.

In certain embodiments, oligonucleotide decoys comprise flanking sequences located at each end of the decoy sequence. Flanking sequences can be 1, 2, 3, 4, 5, 6, or more base pairs in length. In general, flanking sequences are two to five base pairs in length. In preferred embodiments, 5′ flanking sequences starts with a G/C base pair and 3′ flanking sequences terminate in a G/C base pair. In preferred embodiments, flanking sequences do not form part of a transcription factor binding site or do not interact with or bind to transcription factors. In other embodiments, flanking sequences form weak interactions with transcription factors bound to an adjacent transcription factor binding site.

In certain embodiments, oligonucleotide decoys are generally at least 10, 11, 12, 13, 14, 15, or more base pairs in length. In related embodiments, oligonucleotide decoys are generally less than 65, 60, 55, 50, or 45 base pairs in length. In some embodiments, oligonucleotide decoys are about 20 to 40 base pairs in length. In other embodiments, oligonucleotide decoys are about 20 to 35, 25 to 40, or 25 to 35 base pairs in length.

In certain embodiments, the oligonucleotide decoys comprise: (a) a sequence selected from the group consisting of SEQ ID NOs.: 1-40, 42, 45 and 47-53; or (b) a sequence having at least 50%, 55%, 60%, 65%, 70%, 75%, 80%, 85%, 88%, 89%, 90%, 91%, 92%, 93%, 94%, 95%, 96%, 97%, 98%, 99%, or 100% identity with a sequence selected from the group consisting of SEQ ID NOs.: 1-40, 42, 45 and 47-53. In related embodiments, the oligonucleotide decoys comprise a sequence having at least 90% identity with a sequence selected from the group consisting of SEQ ID NOs.: 1-39, 42, 45 and 47-52. In other embodiments, the oligonucleotide decoys comprise a sequence having at least 85% identity with a sequence selected from the group consisting of SEQ ID NOs.: 1-17, 19-39, 42, 45 and 47-53. In other embodiments, the oligonucleotide decoys comprise a sequence having at least 80% identity with a sequence selected from the group consisting of SEQ ID NOs.: 1-5, 7-17, 19-39, 42, 45 and 47-53. In other embodiments, the oligonucleotide decoys comprise a sequence having at least 75% identity with a sequence selected from the group consisting of SEQ ID NOs.: 1-4, 7-9, 13, 15-17, 19-23, 26-39, 45, 48, 50, 51 and 53. In other embodiments, the oligonucleotide decoys comprise a sequence having at least 70% identity with a sequence selected from the group consisting of SEQ ID NOs.: 1-3, 7-9, 13, 15-17, 19-23, 26, 28, 30, 32, 34-36, 38-39 and 48. In other embodiments, the oligonucleotide decoys comprise a sequence having at least 65% identity with a sequence selected from the group consisting of SEQ ID NOs.: 2-3, 9, 13, 15-16, 19-23, 26, 28, 30, 32, 34-36, 38 and 39. In other embodiments, the oligonucleotide decoys comprise a sequence having at least 60% identity with a sequence selected from the group consisting of SEQ ID NOs.: 2, 13, 15-16, 21, 23, 26, 30, 32, 34-36, 38 and 39. In still other embodiments, the oligonucleotide decoys comprise a sequence having at least 55% identity with a sequence selected from the group consisting of SEQ ID NOs.: 16, 23, 30, 32, 34, 35, 38 and 39. In still other embodiments, the oligonucleotide decoys comprise a sequence having at least 50% identity with a sequence selected from the group consisting of SEQ ID NOs.: 30, 32, 35, and 38.

In certain embodiments, an oligonucleotide decoy comprises a double-stranded sequence represented by formula (1):

(1)
5′-S₁n₂n₃n₄n₅A₆T₇D₈B₉N₁₀d₁₁d₁₂n₁₃n₁₄n₁₅n₁₆n₁₇A₁₈T₁₉D₂₀ . . .
. . . B₂₁N₂₂H₂₃H₂₄n₂₅n₂₆n₂₇n₂8n₂₉n₃₀S₃₁-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “D” can be an A, G, or T nucleotide, “B” can be a C, G, or T nucleotide, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (1) has at least about 70%, 75%, 80%, 85%, 88%, 90%, 91%, 92%, 93%, 94%, 95%, 96%, 97%, 98%, or 99% sequence identity to the nucleotide sequence of SEQ ID NO.: 1. Such oligonucleotide decoys can bind to POU2F1 transcription factor. In certain embodiments, such oligonucleotide decoys can bind to one or more transcription factors closely related to POU2F1 transcription factor, such as POU2F2, POU3F1-2, and POU5F1.

In certain embodiments, an oligonucleotide decoy represented by formula (1) comprises a deletion of one or more (e.g., 1, 2, 3, 4, 5, 6, or 7) nucleotides selected from the group consisting of d₁₁, d₁₂, n₁₃, n₁₄, n₁₅, n₁₆, and n₁₇. In certain embodiments, oligonucleotide decoys comprising a deletion of one or more nucleotides selected from the group consisting of d₁₁, d₁₂, n₁₃, n₁₄, n₁₅, n₁₆, and n₁₇ have at least 70% identity to the nucleotide sequence of SEQ ID NO.: 1.

In certain embodiments, an oligonucleotide decoy comprises a double-stranded sequence represented by formula (2):

(2)
5′-S₁n₂n₃n₄n₅n₆Y₇C₈V₉Y₁₀R₁₁N₁₂G₁₃n₁₄n₁₅c₁₆v₁₇y₁₈d₁₉b₂₀ . . .
. . . g₂₁y₂₂C₂₃V₂₄Y₂₅R₂₆B₂₇G₂₈R₂₉n₃₀n₃₁n₃₂n₃₃n₃₄n₃₅S₃₆-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “D” can be an A, G, or T nucleotide, “B” can be a C, G, or T nucleotide, “R” can be a G or an A, “V” can be an A, C, or G, “Y” can be a C or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (2) has at least about 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 2. Such oligonucleotide decoys can bind to USF1 transcription factor. In certain embodiments, such oligonucleotide decoys can bind to one or more transcription factors closely related to USF1 transcription factor, such as USF2.

In certain embodiments, an oligonucleotide decoy represented by formula (2) comprises a deletion of one or more (e.g., 1, 2, 3, 4, 5, 6, 7, 8 or 9) nucleotides selected from the group consisting of n₁₄, n₁₅, c₁₆, v₁₇, y₁₈, d₁₉, b₂₀, g₂₁, and y₂₂. In certain embodiments, oligonucleotide decoys comprising a deletion of one or more nucleotides selected from the group consisting of n₁₄, n₁₅, c₁₆, v₁₇, y₁₈, d₁₉, b₂₀, g₂₁, and y₂₂ have at least 60% identity to the nucleotide sequence of SEQ ID NO.: 2.

In certain embodiments, an oligonucleotide decoy comprises a double-stranded sequence represented by formula (3):

(3)
5′-S₁n₂n₃W₄W₅G₆S₇G₈K₉R₁₀G₁₁G₁₂M₁₃n₁₄n₁₅n₁₆w₁₇w₁₈
w₁₉g₂₀......S₂₁g₂₂K₂₃R₂₄G₂₅G₂₆M₂₇D₂₈n₂₉n₃₀n₃₁n₃₂
n₃₃S₃₄-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, ‘W’ can be an A or a T, “D” can be an A, G, or T nucleotide, “R” can be a G or an A, “K” can be a T or a G, “M” can be a C or an A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (3) has at least about 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 3. Such oligonucleotide decoys can bind to EGR1 transcription factor. In certain embodiments, such oligonucleotide decoys can bind to one or more transcription factors closely related to EGR1 transcription factor, such as EGR2-4.

In certain embodiments, an oligonucleotide decoy represented by formula (3) comprises a deletion of one or more (e.g., 1, 2, 3, 4, 5, 6, 7, 8 or 9) nucleotides selected from the group consisting of n₁₄, n₁₅, n₁₆, w₁₇, w₁₈, w₁₉, g₂₀, s₂₁, and g₂₂. In certain embodiments, oligonucleotide decoys comprising a deletion of one or more nucleotides selected from the group consisting of n₁₄, n₁₅, n₁₆, w₁₇, w₁₈, w₁₉, g₂₀, s₂₁, and g₂₂ have at least 65% identity to the nucleotide sequence of SEQ ID NO.: 3.

In certain embodiments, an oligonucleotide decoy comprises a double-stranded sequence represented by formula (4):

(4)
5′-S₁n₂n₃n₄n₅n₆n₇T₈K₉A₁₀S₁₁S₁₂b₁₃m₁₄n₁₅n₁₆T₁₇K₁₈
A₁₉S₂₀......S₂₁B₂₂M₂₃N₂₄n₂₅n₂₆n₂₇n₂₈S₂₉-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “B” can be a C, G or T, “K” can be a T or a G, “M” can be a C or an A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (4) has at least about 75%, 80%, 85%, 88%, 90%, 91%, 92%, 93%, 94%, 95%, 96%, 97%, 98%, or 99% sequence identity to the nucleotide sequence of SEQ ID NO.: 4. Such oligonucleotide decoys can bind to CREB1 transcription factor. In certain embodiments, such oligonucleotide decoys can bind to one or more transcription factors closely related to CREB1 transcription factor, such as CREB3-5 and ATF1-7.

In certain embodiments, an oligonucleotide decoy represented by formula (4) comprises a deletion of one or more (e.g., 1, 2, 3 or 4) nucleotides selected from the group consisting of b₁₃, m₁₄, n₁₅, and nib. In certain embodiments, oligonucleotide decoys comprising a deletion of one or more nucleotides selected from the group consisting of b₁₃, m₁₄, n₁₅, and n₁₆ have at least 75% identity to the nucleotide sequence of SEQ ID NO.: 4.

In certain embodiments, an oligonucleotide decoy comprises a double-stranded sequence represented by formula (5):

(5)
5′-S₁S₂n₃n₄n₅n₆T₇G₈A₉S₁₀k₁₁n₁₂h₁₃r₁₄r₁₅r₁₆t₁₇G₁₈
A₁₉S₂₀......K₂₁N₂₂H₂₃r₂₄r₂₅n₂₆n₂₇n₂₈S₂₉S₃₀-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “R” can be a G or an A, “K” can be a T or a G, “H” can be a C, T or an A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (5) has at least about 80%, 85%, 88%, 90%, 91%, 92%, 93%, 94%, 95%, 96%, 97%, 98%, or 99% sequence identity to the nucleotide sequence of SEQ ID NO.: 5. Such oligonucleotide decoys can bind to AP1/JUN transcription factors. In certain embodiments, such oligonucleotide decoys can bind to one or more transcription factors closely related to AP1/JUN transcription factors, such as AP1/JUN-B, -D and AP1/FOS.

In certain embodiments, an oligonucleotide decoy represented by formula (5) comprises a deletion of one or more (e.g., 1, 2, 3, 4, 5, 6 or 7) nucleotides selected from the group consisting of k₁₁, n₁₂, h₁₃, r₁₄, r₁₅, r₁₆, and t₁₇. In certain embodiments, oligonucleotide decoys comprising a deletion of one or more nucleotides selected from the group consisting of k₁₁, n₁₂, h₁₃, r₁₄, r₁₅, r₁₆, and t₁₇ have at least 80% identity to the nucleotide sequence of SEQ ID NO.: 5.

In certain embodiments, an oligonucleotide decoy comprises a double-stranded sequence represented by formula (6):

(6)
5′-S₁n₂n₃n₄n₅w₆w₇w₈G₉A₁₀T₁₁T₁₂K₁₃T₁₄s₁₅s₁₆a₁₇a₁₈
k₁₉S₂₀......n₂₁g₂₂A₂₃T₂₄T₂₅K₂₆T₂₇C₂₈S₂₉A₃₀A₃₁K₃₂
S₃₃n₃₄n₃₅n₃₆S₃₇-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be A or T, “K” can be a T or a G, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (6) has at least about 85%, 88%, 90%, 91%, 92%, 93%, 94%, 95%, 96%, 97%, 98%, or 99% sequence identity to the nucleotide sequence of SEQ ID NO.: 6. Such oligonucleotide decoys can bind to CEBPA transcription factor. In certain embodiments, such oligonucleotide decoys can bind to one or more transcription factors closely related to CEBPA transcription factor, such as CEBP-B, -D, -E, -G, -Z.

In certain embodiments, an oligonucleotide decoy represented by formula (6) comprises a deletion of one or more (e.g., 1, 2, 3, 4, 5, 6, 7 or 8) nucleotides selected from the group consisting of s₁₅, s₁₆, a₁₇, a₁₈, k₁₉, s₂₀, n₂₁, and g₂₂. In certain embodiments, oligonucleotide decoys comprising a deletion of one or more nucleotides selected from the group consisting of s₁₅, s₁₆, a₁₇, a₁₈, k₁₉, s₂₀, n₂₁, and g₂₂ have at least 85% identity to the nucleotide sequence of SEQ ID NO.: 6.

In certain embodiments, an oligonucleotide decoy comprises a double-stranded sequence represented by formula (7):

(7)
5′-S₁n₂n₃n₄n₅n₆g₇g₈a₉t₁₀r₁₁t₁₂C₁₃C₁₄A₁₅T₁₆A₁₇T₁₈
T₁₉A₂₀......G₂₁G₂₂a₂₃g₂₄a₂₅t₂₆n₂₇n₂₈n₂₉n₃₀w₃₁w₃₂
s₃₃S₃₄-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or T, Y can be a C or T, “R” can be a G or A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (7) has at least about 70%, 75%, 80%, 85%, 88%, 90%, 91%, 92%, 93%, 94%, 95%, 96%, 97%, 98%, or 99% sequence identity to the nucleotide sequence of SEQ ID NO.: 7. Such oligonucleotide decoys can bind to SRF transcription factor. In certain embodiments, such oligonucleotide decoys can bind to one or more transcription factors closely related to SRF transcription factor, such as ELK1.

In certain embodiments, an oligonucleotide decoy represented by formula (7) comprises a deletion of one or more (e.g., 1, 2, 3, 4, 5, 6, 7, 8, 9, 10, 11, 12, 13, 14, 15, 16 or 17) nucleotides selected from the group consisting of g₇, g₈, a₉, t₁₀, r₁₁, t₁₂, a₂₃, g₂₄, a₂₅, t₂₆, n₂₇, n₂₈, n₂₉, n₃₀, w₃₁, w₃₂ and s₃₃. In certain embodiments, oligonucleotide decoys comprising a deletion of one or more nucleotides selected from the group consisting of g₇, g₈, a₉, t₁₀, r₁₁, t₁₂, a₂₃, g₂₄, a₂₅, t₂₆, n₂₇, n₂₈, n₂₉, n₃₀, w₃₁, w₃₂ and s₃₃ have at least 70% identity to the nucleotide sequence of SEQ ID NO.: 7.

In certain embodiments, an oligonucleotide decoy comprises a double-stranded sequence represented by formula (8):

(8)
5′-S₁n₂n₃n₄n₅C₆A₇G₈G₉A₁₀d₁₁d₁₂d₁₃d₁₄d₁₅d₁₆d₁₇d₁₈
d₁₉T₂₀......C₂₁C₂₂A₂₃T₂₄A₂₅T₂₆T₂₇A₂₈G₂₉n₃₀n₃₁n₃₂
n₃₃S₃₄-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “D” can be an A, T or G, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (8) has at least about 70%, 75%, 80%, 85%, 88%, 90%, 91%, 92%, 93%, 94%, 95%, 96%, 97%, 98%, or 99% sequence identity to the nucleotide sequence of SEQ ID NO.: 8. Such oligonucleotide decoys can bind to SRF transcription factor. In certain embodiments, such oligonucleotide decoys can bind to one or more transcription factors closely related to SRF transcription factor, such as ETS1.

In certain embodiments, an oligonucleotide decoy represented by formula (8) comprises a deletion of one or more (e.g., 1, 2, 3, 4, 5, 6, 7, 8 or 9) nucleotides selected from the group consisting of d₁₁, d₁₂, d₁₃, d₁₄, d₁₅, d₁₆, d₁₇, d₁₈ and d₁₉. In certain embodiments, oligonucleotide decoys comprising a deletion of one or more nucleotides selected from the group consisting of d₁₁, d₁₂, d₁₃, d₁₄, d₁₅, d₁₆, d₁₇, d₁₈ and d₁₉ have at least 70% identity to the nucleotide sequence of SEQ ID NO.: 8.

In certain embodiments, an oligonucleotide decoy comprises a double-stranded sequence represented by formula (9):

(9)
5′-S₁n₂n₃n₄n₅C₆T₇A₈W₉A₁₀M₁₁W₁₂T₁₃A₁₄A₁₅n₁₆n₁₇n₁₈
n₁₉c₂₀......t₂₁A₂₂W₂₃A₂₄A₂₅A₂₆T₂₇A₂₈A₂₉A₃₀A₃₁n₃₂
n₃₃n₃₄S₃₅-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or an T, “M” can be a C or an A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (9) has at least about 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 9. Such oligonucleotide decoys can bind to MEF2A transcription factor. In certain embodiments, such oligonucleotide decoys can bind to one or more transcription factors closely related to MEF2A transcription factor, such as MEF2B-C.

In certain embodiments, an oligonucleotide decoy represented by formula (9) comprises a deletion of one or more (e.g., 1, 2, 3, 4, 5 or 6) nucleotides selected from the group consisting of n₁₆, n₁₇, n₁₈, n₁₉, c₂₀ and t₂₁. In certain embodiments, oligonucleotide decoys comprising a deletion of one or more nucleotides selected from the group consisting of n₁₆, n₁₇, n₁₈, n₁₉, c₂₀ and t₂₁ have at least 65% identity to the nucleotide sequence of SEQ ID NO.: 9.

In certain embodiments, an oligonucleotide decoy comprises a double-stranded sequence represented by formula (10):

(10)
5′-n₁n₂n₃n₄R₅R₆G₇S₈C₉S₁₀K₁₁r₁₂r₁₃n₁₄n₁₅n₁₆r₁₇r₁₈
G₁₉S₂₀......C₂₁K₂₂R₂₃R₂₄N₂₅n₂₆n₂₇n₂₈n₂₉n₃₀-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “K” can be a T or a G, “R” can be a G or an A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (10) has at least about 80%, 85%, 88%, 90%, 91%, 92%, 93%, 94%, 95%, 96%, 97%, 98%, or 99% sequence identity to the nucleotide sequence of SEQ ID NO.: 10. Such oligonucleotide decoys can bind to SP1 transcription factor. In certain embodiments, such oligonucleotide decoys can bind to one or more transcription factors closely related to SP1 transcription factor, such as SP2-8.

In certain embodiments, an oligonucleotide decoy represented by formula (10) comprises a deletion of one or more (e.g., 1, 2, 3, 4, 5, 6 or 7) nucleotides selected from the group consisting of r₁₂, r₁₃, n₁₄, n₁₅, n₁₆, r₁₇, and r₁₈. In certain embodiments, oligonucleotide decoys comprising a deletion of one or more nucleotides selected from the group consisting of n₁₆, n₁₇, n₁₈, n₁₉, c₂₀ and t₂₁ have at least 80% identity to the nucleotide sequence of SEQ ID NO.: 10.

In certain embodiments, an oligonucleotide decoy comprises a double-stranded sequence represented by formula (11):

(11)
5′-n₁n₂n₃n₄n₅G₆G₇C₈G₉G₁₀G₁₁G₁₂s₁₃S₁₄S₁₅S₁₆S₁₇S₁₈
S₁₉S₂₀......S₂₁S₂₂S₂₃C₂₄G₂₅G₂₆G₂₇C₂₈G₂₉G₃₀T₃₁T₃₂
T₃₃A₃₄C₃₅-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (11) has at least about 80%, 85%, 88%, 90%, 91%, 92%, 93%, 94%, 95%, 96%, 97%, 98%, or 99% sequence identity to the nucleotide sequence of SEQ ID NO.: 11. Such oligonucleotide decoys can bind to SP1 transcription factor. In certain embodiments, such oligonucleotide decoys can bind to one or more transcription factors closely related to SP1 transcription factor, such as SP2-8.

In certain embodiments, an oligonucleotide decoy represented by formula (11) comprises a deletion of one or more (e.g., 1, 2, 3, 4, 5, 6, 7, 8, 9, 10 or 11) nucleotides selected from the group consisting of s₁₃, s₁₄, s₁₅, s₁₆, s₁₇, s₁₈, s₁₉, s₂₀, s₂₁, s₂₂, and s₂₃. In certain embodiments, oligonucleotide decoys comprising a deletion of one or more nucleotides selected from the group consisting of s₁₃, s₁₄, s₁₅, s₁₆, s₁₇, s₁₈, s₁₉, s₂₀, s₂₁, s₂₂, and s₂₃ have at least 80% identity to the nucleotide sequence of SEQ ID NO.: 11.

In certain embodiments, an oligonucleotide decoy comprises a double-stranded sequence represented by formula (12):

(12)
5′-S₁n₂n₃n₄n₅W₆G₇Y₈G₉G₁₀t₁₁d₁₂d₁₃d₁₄d₁₅g₁₆W₁₇G₁₈
Y₁₉G₂₀......G₂₁T₂₂D₂₃D₂₄D₂₅D₂₆n₂₇n₂₈S₂₉-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, Y can be a C or a T, “D” can be an A, T or a G, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (12) has at least about 80%, 85%, 88%, 90%, 91%, 92%, 93%, 94%, 95%, 96%, 97%, 98%, or 99% sequence identity to the nucleotide sequence of SEQ ID NO.: 12. Such oligonucleotide decoys can bind to RUNX1 transcription factor. In certain embodiments, such oligonucleotide decoys can bind to one or more transcription factors closely related to RUNX1 transcription factor, such as RUNX2-3.

In certain embodiments, an oligonucleotide decoy represented by formula (12) comprises a deletion of one or more (e.g., 1, 2, 3, 4, 5 or 6) nucleotides selected from the group consisting of t₁₁, h₁₂, h₁₃, h₁₄, h₁₅, and g₁₆. In certain embodiments, oligonucleotide decoys comprising a deletion of one or more nucleotides selected from the group consisting of t₁₁, h₁₂, h₁₃, h₁₄, h₁₅, and g₁₆ have at least 80% identity to the nucleotide sequence of SEQ ID NO.: 12.

In certain embodiments, an oligonucleotide decoy comprises a double-stranded sequence represented by formula (13):

(13)
5′-S₁n₂n₃n₄n₅T₆T₇G₈G₉G₁₀G₁₁T₁₂C₁₃A₁₄T₁₅A₁₆n₁₇n₁₈
n₁₉n₂₀......C₂₁A₂₂C₂₃A₂₄G₂₅G₂₆A₂₇A₂₈C₂₉C₃₀A₃₁C₃₂
A₃₃n₃₄n₃₅S₃₆-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (13) has at least about 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 13. Such oligonucleotide decoys can bind to RUNX1 transcription factor. In certain embodiments, such oligonucleotide decoys can bind to one or more transcription factors closely related to RUNX1 transcription factor, such as RUNX2-3.

In certain embodiments, an oligonucleotide decoy represented by formula (13) comprises a deletion of one or more (e.g., 1, 2, 3 or 4) nucleotides selected from the group consisting of n₁₇, n₁₈, n₁₉ and n₂₀. In certain embodiments, oligonucleotide decoys comprising a deletion of one or more nucleotides selected from the group consisting of n₁₇, n₁₈, n₁₉ and n₂₀ have at least 60% identity to the nucleotide sequence of SEQ ID NO.: 13.

In certain embodiments, an oligonucleotide decoy comprises a double-stranded sequence represented by formula (14):

(14)
5′-S₁n₂n₃n₄n₅n₆C₇H₈G₉G₁₀A₁₁H₁₂R₁₃y₁₄n₁₅n₁₆n₁₇c₁₈
C₁₉G₂₀......G₂₁A₂₂H₂₃R₂₄Y₂₅n₂₆n₂₇n₂₈n₂₉n₃₀n₃₁
S₃₂-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “R” can be G or A, “H” can be A, T or C, “Y” can be a C or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (14) has at least about 80%, 85%, 88%, 90%, 91%, 92%, 93%, 94%, 95%, 96%, 97%, 98%, or 99% sequence identity to the nucleotide sequence of SEQ ID NO.: 14. Such oligonucleotide decoys can bind to ETS1 transcription factor. In certain embodiments, such oligonucleotide decoys can bind to one or more transcription factors closely related to ETS1 transcription factor, such as ELK1.

In certain embodiments, an oligonucleotide decoy represented by formula (14) comprises a deletion of one or more (e.g., 1, 2, 3, 4 or 5) nucleotides selected from the group consisting of y₁₄, n₁₅, n₁₆, n₁₇ and c₁₈. In certain embodiments, oligonucleotide decoys comprising a deletion of one or more nucleotides selected from the group consisting of y₁₄, n₁₅, n₁₆, n₁₇ and c₁₈ have at least 80% identity to the nucleotide sequence of SEQ ID NO.: 14.

In certain embodiments, an oligonucleotide decoy comprises a double-stranded sequence represented by formula (15):

(15)
5′-S₁n₂n₃M₄W₅W₆G₇G₈A₉A₁₀A₁₁A₁₂n₁₃n₁₄d₁₅w₁₆w₁₇g₁₈
g₁₉a₂₀......a₂₁a₂₂a₂₃n₂₄n₂₅d₂₆W₂₇G₂₈G₂₉A₃₀A₃₁A₃₂
A₃₃n₃₄n₃₅n₃₆n₃₇n₃₈n₃₉S₄₀-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “D” can be an A, G or a T, “W” can be an A or a T, “M” can be C or A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (15) has at least about 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 15. Such oligonucleotide decoys can bind to NFATC1 transcription factor. In certain embodiments, such oligonucleotide decoys can bind to one or more transcription factors closely related to NFATC1 transcription factor, such as NFATC2-4.

In certain embodiments, an oligonucleotide decoy represented by formula (15) comprises a deletion of one or more (e.g., 1, 2, 3, 4, 5, 6, 7, 8, 9, 10, 11, 12, 13, 14 or 15) nucleotides selected from the group consisting of n₁₃, n₁₄, d₁₅, w₁₆, w₁₇, g₁₈, g₁₉, a₂₀, a₁₁, a₂₂, a₂₃, n₂₄, n₂₅, d₂₆ and w₂₇. In certain embodiments, oligonucleotide decoys comprising a deletion of one or more nucleotides selected from the group consisting of n₁₃, n₁₄, d₁₅, w₁₆, w₁₇, g₁₈, g₁₉, a₂₀, a₂₁, a₂₂, a₂₃, n₂₄, n₂₅, d₂₆ and w₂₇ have at least 60% identity to the nucleotide sequence of SEQ ID NO.: 15.

In certain embodiments, an oligonucleotide decoy comprises a double-stranded sequence represented by formula (16):

(16)
5′-S₁n₂n₃n₄n₅n₆C₇A₈C₉T₁₀T₁₁C₁₂C₁₃y₁₄v₁₅m₁₆n₁₇n₁₈
n₁₉y₂₀......v₂₁C₂₂T₂₃T₂₄C₂₅C₂₆T₂₇G₂₈C₂₉n₃₀n₃₁n₃₂
S₃₃-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “V” can be G, A or C, “M” can be C or A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (16) has at least about 55%, 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 16. Such oligonucleotide decoys can bind to ELK1 transcription factor. In certain embodiments, such oligonucleotide decoys can bind to one or more transcription factors closely related to ELK1 transcription factor, such as ETS1.

In certain embodiments, an oligonucleotide decoy represented by formula (16) comprises a deletion of one or more (e.g., 1, 2, 3, 4, 5, 6, 7 or 8) nucleotides selected from the group consisting of y₁₄, v₁₅, m₁₆, n₁₇, n₁₅, n₁₉, y₂₀ and v₂₁. In certain embodiments, oligonucleotide decoys comprising a deletion of one or more nucleotides selected from the group consisting of y₁₄, v₁₅, m₁₆, n₁₇, n₁₈, n₁₉, y₂₀ and v₂₁ have at least 55% identity to the nucleotide sequence of SEQ ID NO.: 16.

In certain embodiments, an oligonucleotide decoy comprises a double-stranded sequence represented by formula (17):

(17)
5′-S₁n₂n₃n₄n₅n₆C₇T₈A₉T₁₀A₁₁A₁₂A₁₃T₁₄g₁₅g₁₆c₁₇c₁₈
t₁₉A₂₀......T₂₁A₂₂A₂₃A₂₄T₂₅G₂₆g₂₇g₂₈g₂₉g₃₀g₃₁g₃₂
S₃₃-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (17) has at least about 70%, 75%, 80%, 85%, 88%, 90%, 91%, 92%, 93%, 94%, 95%, 96%, 97%, 98%, or 99% sequence identity to the nucleotide sequence of SEQ ID NO.: 17. Such oligonucleotide decoys can bind to ternary complex factors. In certain embodiments, such oligonucleotide decoys can bind to one or more transcription factors closely related to ternary complex factors, such as SRF.

In certain embodiments, an oligonucleotide decoy represented by formula (17) comprises a deletion of one or more (e.g., 1, 2, 3, 4 or 5) nucleotides selected from the group consisting of g₁₅, g₁₆, c₁₇, c₁₈ and t₁₉. In certain embodiments, oligonucleotide decoys comprising a deletion of one or more nucleotides selected from the group consisting of g₁₅, g₁₆, c₁₇, c₁₈ and t₁₉ have at least 70% identity to the nucleotide sequence of SEQ ID NO.: 17.

In certain embodiments, an oligonucleotide decoy comprises a double-stranded sequence represented by formula (18):

(18)
5′-S₁n₂n₃n₄n₅n₆n₇W₈W₉C₁₀G₁₁C₁₂G₁₃G₁₄w₁₅w₁₆g₁₇g₁₈w₁₉
w₂₀ . . . . . . w₂₁C₂₂C₂₃G₂₄G₂₅W₂₆W₂₇n₂₈n₂₉n₃₀n₃₁
n₃₂S₃₃-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can an A or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (18) has at least about 90%, 91%, 92%, 93%, 94%, 95%, 96%, 97%, 98%, or 99% sequence identity to the nucleotide sequence of SEQ ID NO.: 18. Such oligonucleotide decoys can bind to STAT1 transcription factor. In certain embodiments, such oligonucleotide decoys can bind to one or more transcription factors closely related to STAT1 transcription factor, such as STAT2-6.

In certain embodiments, an oligonucleotide decoy represented by formula (18) comprises a deletion of one or more (e.g., 1, 2, 3, 4, 5, 6 or 7) nucleotides selected from the group consisting of w₁₅, w₁₆, g₁₇, g₁₈, w₁₈, w₂₀ and w₂₁. In certain embodiments, oligonucleotide decoys comprising a deletion of one or more nucleotides selected from the group consisting of w₁₅, w₁₆, g₁₇, g₁₈, w₁₉, w₂₀ and w₂₁ have at least 90% identity to the nucleotide sequence of SEQ ID NO.: 18.

In certain embodiments, an oligonucleotide decoy comprises a double-stranded sequence represented by formula (19):

(19)
5′-S₁n₂n₃n₄T₅G₆C₇C₈T₉T₁₀A₁₁T₁₂C₁₃T₁₄c₁₅t₁₆n₁₇n₁₈g₁₉
g₂₀ . . . . . . G₂₁A₂₂T₂₃A₂₄A₂₅S₂₆n₂₇n₂₈n₂₉n₃₀
S₃₁-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (19) has at least about 65%, 70%, 75%, 80%, 85%, 90%, 91%, 92%, 93%, 94%, 95%, 96%, 97%, 98%, or 99% sequence identity to the nucleotide sequence of SEQ ID NO.: 19. Such oligonucleotide decoys can bind to GATA1 transcription factor. In certain embodiments, such oligonucleotide decoys can bind to one or more transcription factors closely related to GATA1 transcription factor, such as GATA2-4.

In certain embodiments, an oligonucleotide decoy represented by formula (19) comprises a deletion of one or more (e.g., 1, 2, 3, 4, 5 or 6) nucleotides selected from the group consisting of c₁₅, t₁₆, n₁₇, n₁₈, g₁₉ and g₂₀. In certain embodiments, oligonucleotide decoys comprising a deletion of one or more nucleotides selected from the group consisting of c₁₅, t₁₆, n₁₇, n₁₈, g₁₉ and g₂₀ have at least 65% identity to the nucleotide sequence of SEQ ID NO.: 19.

In certain embodiments, an oligonucleotide decoy comprises a double-stranded sequence represented by formula (20):

(20)
5′-S₁n₂n₃n₄n₅n₆T₇G₈A₉A₁₀T₁₁w₁₂w₁₃g₁₄a₁₅g₁₆g₁₇a₁₈a₁₉
a₂₀ . . . . . . a₂₁w₂₂w₂₃G₂₄C₂₅A₂₆T₂₇G₂₈C₂₉n₃₀n₃₁
S₃₂-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can an A or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (20) has at least about 65%, 70%, 75%, 80%, 85%, 90%, 91%, 92%, 93%, 94%, 95%, 96%, 97%, 98%, or 99% sequence identity to the nucleotide sequence of SEQ ID NO.: 20. Such oligonucleotide decoys can bind to ELF1 transcription factor. In certain embodiments, such oligonucleotide decoys can bind to one or more transcription factors closely related to ELF1 transcription factor, such as POU1F1.

In certain embodiments, an oligonucleotide decoy represented by formula (20) comprises a deletion of one or more (e.g., 1, 2, 3, 4, 5, 6, 7, 8, 9, 10, 11 or 12) nucleotides selected from the group consisting of w₁₂, w₁₃, g₁₄, a₁₅, g₁₆, g₁₇, a₁₈, a₁₉, a₂₀, a₂₁, w₂₂ and w₂₃. In certain embodiments, oligonucleotide decoys comprising a deletion of one or more nucleotides selected from the group consisting of w₁₂, w₁₃, g₁₄, a₁₅, g₁₆, g₁₇, a₁₈, a₁₉, a₂₀, a₂₁, w₂₂ and w₂₃ have a t least 65% identity to the nucleotide sequence of SEQ ID NO.: 20

In certain embodiments, an oligonucleotide decoy comprises a double-stranded sequence represented by formula (21):

(21)
5′-S₁n₂n₃n₄n₅G₆A₇G₈A₉T₁₀T₁₁k₁₂c₁₃a₁₄c₁₅n₁₆n₁₇n₁₈
g₁₉a₂₀ . . . . . . g₂₁a₂₂t₂₃T₂₄K₂₅C₂₆A₂₇C₂₈n₂₉
n₃₀n₃₁n₃₂S₃₃-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “K” can be a G or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (21) has at least about 60%, 65%, 70%, 75%, 80%, 85%, 90%, 91%, 92%, 93%, 94%, 95%, 96%, 97%, 98%, or 99% sequence identity to the nucleotide sequence of SEQ ID NO.: 21. Such oligonucleotide decoys can bind to “nuclear factor—granulocyte/macrophage a” transcription factors. In certain embodiments, such oligonucleotide decoys can bind to one or more transcription factors closely related to “nuclear factor—granulocyte/macrophage a” transcription factors, such as “nuclear factor—granulocyte/macrophage b-c”.

In certain embodiments, an oligonucleotide decoy represented by formula (21) comprises a deletion of one or more (e.g., 1, 2, 3, 4, 5, 6, 7, 8, 9, 10, 11 or 12) nucleotides selected from the group consisting of k₁₂, c₁₃, a₁₄, c₁₅, n₁₆, n₁₇, n₁₈, g₁₉, a₂₀, g₂₁, a₂₂ and t₂₃. In certain embodiments, oligonucleotide decoys comprising a deletion of one or more nucleotides selected from the group consisting of k₁₂, c₁₃, a₁₄, c₁₅, n₁₆, n₁₇, n₁₈, g₁₉, a₂₀, g₂₁, a₂₂ and t₂₃ have at least 60% identity to the nucleotide sequence of SEQ ID NO.: 21.

In certain embodiments, an oligonucleotide decoy comprises a double-stranded sequence represented by formula (22):

(22)
5′-S₁n₂n₃n₄n₅K₆C₇M₈T₉W₁₀A₁₁W₁₂t₁₃r₁₄m₁₅w₁₆n₁₇r₁₈
m₁₉w₂₀ . . . . . . K₂₁C₂₂M₂₃T₂₄W₂₅A₂₆W₂₇T₂₈n₂₉
n₃₀n₃₁S₃₂-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can an A or a T, “K” can be a G or a T, “M” can be an A or a C, “R” can be an A or a G, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (22) has at least about 65%, 70%, 75%, 80%, 85%, 90%, 91%, 92%, 93%, 94%, 95%, 96%, 97%, 98%, or 99% sequence identity to the nucleotide sequence of SEQ ID NO.: 22. Such oligonucleotide decoys can bind to POU4F1 transcription factor. In certain embodiments, such oligonucleotide decoys can bind to one or more transcription factors closely related to POU4F1 transcription factor, such as POU4F2-3.

In certain embodiments, an oligonucleotide decoy represented by formula (22) comprises a deletion of one or more (e.g., 1, 2, 3, 4, 5, 6, 7 or 8) nucleotides selected from the group consisting of t₁₃, r₁₄, m₁₅, w₁₆, n₁₇, r₁₈, m₁₉ and w₂₀. In certain embodiments, oligonucleotide decoys comprising a deletion of one or more nucleotides selected from the group consisting of t₁₃, r₁₄, m₁₅, w₁₆, n₁₇, r₁₈, m₁₉ and w₂₀ have at least 65% identity to the nucleotide sequence of SEQ ID NO.: 22.

In certain embodiments, an oligonucleotide decoy comprises a double-stranded sequence represented by formula (23):

(23)
5′-S₁n₂n₃n₄A₅G₆K₇Y₈A₉A₁₀D₁₁N₁₂D₁₃T₁₄h₁₅h₁₆h₁₇n₁₈
n₁₉n₂₀ . . . . . . h₂₁h₂₂H₂₃Y₂₄A₂₅A₂₆D₂₇N₂₈D₂₉
T₃₀W₃₁V₃₂M₃₃t₃₄g₃₅C₃₆-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “V” can be G, A or C, “K” can be T or G, “D” can be G, A or T, “H” can be A, T or C, “W” can be A or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (23) has at least about 55%, 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 23. Such oligonucleotide decoys can bind to HNF1A transcription factor. In certain embodiments, such oligonucleotide decoys can bind to one or more transcription factors closely related to HNF1A transcription factor, such as HNF1B-C.

In certain embodiments, an oligonucleotide decoy represented by formula (23) comprises a deletion of one or more (e.g., 1, 2, 3, 4, 5, 6, 7 or 8) nucleotides selected from the group consisting of h₁₅, h₁₆, h₁₇, n₁₈, n₁₉, n₂₀, h₂₁ and h₂₂. In certain embodiments, oligonucleotide decoys comprising a deletion of one or more nucleotides selected from the group consisting of h₁₅, h₁₆, h₁₇, n₁₈, n₁₉, n₂₀, h₂₁ and h₂₂ have at least 55% identity to the nucleotide sequence of SEQ ID NO.: 23.

In certain embodiments, an oligonucleotide decoy comprises a double-stranded sequence represented by formula (24):

(24)
5′-S₁n₂n₃n₄n₅A₆A₇T₈A₉A₁₀t₁₁n₁₂n₁₃a₁₄t₁₅T₁₆A₁₇T₁₈
T₁₉w₂₀ . . . . . . w₂₁n₂₂n₂₃n₂₄S₂₅-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (24) has at least about 80%, 85%, 90%, 91%, 92%, 93%, 94%, 95%, 96%, 97%, 98%, or 99% sequence identity to the nucleotide sequence of SEQ ID NO.: 24. Such oligonucleotide decoys can bind to ZFHX3 transcription factor. In certain embodiments, such oligonucleotide decoys can bind to one or more transcription factors closely related to ZFHX3 transcription factor, such as ZFHX-2, -4.

In certain embodiments, an oligonucleotide decoy represented by formula (24) comprises a deletion of one or more (e.g., 1, 2, 3, 4 or 5) nucleotides selected from the group consisting of t₁₁, n₁₂, n₁₃, a₁₄ and t₁₅. In certain embodiments, oligonucleotide decoys comprising a deletion of one or more nucleotides selected from the group consisting of t₁₁, n₁₂, n₁₃, a₁₄ and t₁₅ have at least 80% identity to the nucleotide sequence of SEQ ID NO.: 24.

In certain embodiments, an oligonucleotide decoy comprises a double-stranded sequence represented by formula (25):

(25)
5′-S₁n₂n₃n₄S₅D₆H₇W₈M₉S₁₀H₁₁k₁₂w₁₃w₁₄m₁₅c₁₆s₁₇s₁₈d₁₉
h₂₀ . . . . . . w₂₁m₂₂s₂₃h₂₄K₂₅W₂₆W₂₇M₂₈C₂₉S₃₀n₃₁
n₃₂n₃₃n₃₄S₃₅-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or T, “D” can be A, G or T, “H” can be A, C or T, “M” can be A or C, “K” can be G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (25) has at least about 80%, 85%, 90%, 91%, 92%, 93%, 94%, 95%, 96%, 97%, 98%, or 99% sequence identity to the nucleotide sequence of SEQ ID NO.: 25. Such oligonucleotide decoys can bind to IRF1 transcription factor. In certain embodiments, such oligonucleotide decoys can bind to one or more transcription factors closely related to IRF1 transcription factor, such as IRF2.

In certain embodiments, an oligonucleotide decoy represented by formula (25) comprises a deletion of one or more (e.g., 1, 2, 3, 4, 5, 6, 7, 8, 9, 10, 11, 12 or 13) nucleotides selected from the group consisting of k₁₂, w₁₃, w₁₄, m₁₅, c₁₆, s₁₇, s₁₈, d₁₉, h₂₀, w₂₁, m₂₂, s₂₃ and h₂₄. In certain embodiments, oligonucleotide decoys comprising a deletion of one or more nucleotides selected from the group consisting of k₁₂, w₁₃, w₁₄, m₁₅, c₁₆, s₁₇, s₁₈, d₁₉, h₂₀, w₂₁, m₂₂, s₂₃ and h₂₄ have at least 80% identity to the nucleotide sequence of SEQ ID NO.: 25.

In certain embodiments, an oligonucleotide decoy comprises a double-stranded sequence represented by formula (26):

(26)
5′-S₁n₂n₃n₄y₅k₆g₇y₈k₉G₁₀A₁₁Aₗ₂y₁₃h₁₄b₁₅b₁₆n₁₇n₁₈
n₁₉y₂₀ . . . . . . h₂₁b₂₂b₂₃k₂₄G₂₅A₂₆A₂₇T₂₈A₂₉
T₃₀C₃₁n₃₂n₃₃S₃₄-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “V” can be G, A or C, “K” can be T or G, “D” can be G, A or T, “H” can be A, T or G, “B” can be C, G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (26) has at least about 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 26. Such oligonucleotide decoys can bind to TEAD1 transcription factor. In certain embodiments, such oligonucleotide decoys can bind to one or more transcription factors closely related to TEAD1 transcription factor, such as TEAD2-4.

In certain embodiments, an oligonucleotide decoy represented by formula (26) comprises a deletion of one or more (e.g., 1, 2, 3, 4, 5, 6, 7, 8, 9, 10, 11 or 12) nucleotides selected from the group consisting of y₁₃, h₁₄, h₁₅, h₁₆, n₁₇, n₁₈, n₁₉, y₂₀, h₂₁, b₂₂, b₂₃ and k₂₄. In certain embodiments, oligonucleotide decoys comprising a deletion of one or more nucleotides selected from the group consisting of y₁₃, h₁₄, b₁₅, b₁₆, n₁₇, n₁₈, n₁₉, y₂₀, h₂₁, b₂₂, b₂₃ and k₂₄ have at least 60% identity to the nucleotide sequence of SEQ ID NO.: 26.

In certain embodiments, an oligonucleotide decoy comprises a double-stranded sequence represented by formula (27):

(27)
5′-S₁n₂n₃n₄T₅A₆T₇A₈W₉w₁₀w₁₁n₁₂n₁₃d₁₄n₁₅t₁₆a₁₇t₁₈
A₁₉W₂₀ . . . . . . w₂₁w₂₂n₂₃n₂₄w₂₅W₂₆T₂₇A₂₈A₂₉
D₃₀W₃₁n₃₂n₃₃n₃₄n₃₅n₃₆S₃₇-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, “D” can be an A, G or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (27) has at least about 75%, 80%, 85%, 90%, 91%, 92%, 93%, 94%, 95%, 96%, 97%, 98%, or 99% sequence identity to the nucleotide sequence of SEQ ID NO.: 27. Such oligonucleotide decoys can bind to TBP transcription factor. In certain embodiments, such oligonucleotide decoys can bind to one or more transcription factors closely related to TBP transcription factor, such as TBPL1-2.

In certain embodiments, an oligonucleotide decoy represented by formula (27) comprises a deletion of one or more (e.g., 1, 2, 3, 4, 5, 6, 7, 8, 9, 10, 11, 12, 13 or 14) nucleotides selected from the group consisting of w₁₀, w₁₁, n₁₂, n₁₃, d₁₄, n₁₅, t₁₆, a₁₇, t₁₈, w₂₁, w₂₂, n₂₃, n₂₄, and w₂₅. In certain embodiments, oligonucleotide decoys comprising a deletion of one or more nucleotides selected from the group consisting of w₁₀, w₁₁, n₁₂, n₁₃, d₁₄, n₁₅, t₁₆, a₁₇, t₁₈, w₂₁, w₂₂, n₂₃, n₂₄, and w₂₅ have at least 75% identity to the nucleotide sequence of SEQ ID NO.: 27.

In certain embodiments, an oligonucleotide decoy comprises a double-stranded sequence represented by formula (28):

(28)
5′-S₁n₂n₃n₄T₅A₆T₇A₈A₉W₁₀W₁₁n₁₂n₁₃n₁₄n₁₅w₁₆w₁₇w₁₈A₁₉
A₂₀ . . . . . . W₂₁W₂₂k₂₃n₂₄n₂₅n₂₆n₂₇n₂₈S₂₉-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, “K” can be a G or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (28) has at least about 65%, 70%, 75%, 80%, 85%, 90%, 91%, 92%, 93%, 94%, 95%, 96%, 97%, 98%, or 99% sequence identity to the nucleotide sequence of SEQ ID NO.: 28. Such oligonucleotide decoys can bind to TBP transcription factors. In certain embodiments, such oligonucleotide decoys can bind to one or more transcription factors closely related to TBP transcription factors, such as TBPL1-2.

In certain embodiments, an oligonucleotide decoy represented by formula (28) comprises a deletion of one or more (e.g., 1, 2, 3, 4, 5, 6 or 7) nucleotides selected from the group consisting of n₁₂, n₁₃, n₁₄, n₁₅, w₁₆, w₁₇ and w₁₈. In certain embodiments, oligonucleotide decoys comprising a deletion of one or more nucleotides selected from the group consisting of n₁₂, n₁₃, n₁₄, n₁₅, w₁₆, w₁₇ and w₁₈ have at least 65% identity to the nucleotide sequence of SEQ ID NO.: 28.

In certain embodiments, an oligonucleotide decoy comprises a double-stranded sequence represented by formula (29):

(29)
5′-N₁n₂n₃C₄T₅G₆M₇K₈Y₉K₁₀K₁₁Y₁₂t₁₃m₁₄b₁₅y₁₆C₁₇A₁₈A₁₉
T₂₀ . . . . . . s₂₁d₂₂n₂₃n₂₄n₂₅S₂₆-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “M” can be an A or a C, “K” can be a G or a T, “Y” can be a C or a T, “B” can be a C, G or T, “D” can be an A, G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (29) has at least about 75%, 80%, 85%, 90%, 91%, 92%, 93%, 94%, 95%, 96%, 97%, 98%, or 99% sequence identity to the nucleotide sequence of SEQ ID NO.: 29. Such oligonucleotide decoys can bind to NFYA transcription factor. In certain embodiments, such oligonucleotide decoys can bind to one or more transcription factors closely related to NFYA transcription factor, such as NFYB-C.

In certain embodiments, an oligonucleotide decoy represented by formula (29) comprises a deletion of one or more (e.g., 1, 2, 3 or 4) nucleotides selected from the group consisting of t₁₃, m₁₄, b₁₅ and y₁₆. In certain embodiments, oligonucleotide decoys comprising a deletion of one or more nucleotides selected from the group consisting of t₁₃, m₁₄, bis and y₁₆ have at least 75% identity to the nucleotide sequence of SEQ ID NO.: 29.

In certain embodiments, an oligonucleotide decoy comprises a double-stranded sequence represented by formula (30):

(30)
5′-S₁n₂n₃T₄C₅T₆C₇Y₈G₉A₁₀T₁₁T₁₂G₁₃G₁₄Y₁₅y₁₆h₁₇y₁₈b₁₉
n₂₀ . . . . . . n₂₁n₂₂y₂₃y₂₄h₂₅h₂₆v₂₇G₂₈A₂₉T₃₀T₃₁
G₃₂G₃₃Y₃₄T₃₅C₃₆B₃₇Y₃₈n₃₉S₄₀-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “H” can be A, T or C, “B” can be C, G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (30) has at least about 50%, 55%, 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 30. Such oligonucleotide decoys can bind to NFYA transcription factor. In certain embodiments, such oligonucleotide decoys can bind to one or more transcription factors closely related to NFYA transcription factor, such as NFYB-C.

In certain embodiments, an oligonucleotide decoy represented by formula (30) comprises a deletion of one or more (e.g., 1, 2, 3, 4, 5, 6, 7, 8, 9, 10, 11 or 12) nucleotides selected from the group consisting of y₁₆, h₁₇, y₁₈, b₁₉, b₂₀, b₂₁, n₂₂, y₂₃, y₂₄, h₂₅, h₂₆ and v₂₇. In certain embodiments, oligonucleotide decoys comprising a deletion of one or more nucleotides selected from the group consisting of y₁₆, h₁₇, y₁₈, b₁₉, b₂₀, b₂₁, n₂₂, y₂₃, y₂₄, h₂₅, h₂₆ and v₂₇ have at least 50% identity to the nucleotide sequence of SEQ ID NO.: 30.

In certain embodiments, an oligonucleotide decoy comprises a double-stranded sequence represented by formula (31):

(31)
5′-S₁n₂n₃C₄A₅C₆C₇C₈s₉a₁₀s₁₁s₁₂s₁₃w₁₄s₁₅s₁₆s₁₇w₁₈C₁₉
A₂₀ . . . . . . C₂₁C₂₂C₂₃a₂₄n₂₅n₂₆n₂₇S₂₈-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (31) has at least about 75%, 80%, 85%, 90%, 91%, 92%, 93%, 94%, 95%, 96%, 97%, 98%, or 99% sequence identity to the nucleotide sequence of SEQ ID NO.: 31. Such oligonucleotide decoys can bind to CACCC-box binding factors.

In certain embodiments, an oligonucleotide decoy represented by formula (31) comprises a deletion of one or more (e.g., 1, 2, 3, 4, 5, 6, 7, 8, 9 or 10) nucleotides selected from the group consisting of s₉, a₁₀, s₁₁, s₁₂, s₁₃, w₁₄, s₁₅, s₁₆, s₁₇ and w₁₈. In certain embodiments, oligonucleotide decoys comprising a deletion of one or more nucleotides selected from the group consisting of s₉, a₁₀, s₁₁, s₁₂, s₁₃, w₁₄, s₁₅, s₁₆, s₁₇ and w₁₈ have at least 75% identity to the nucleotide sequence of SEQ ID NO.: 31.

In certain embodiments, an oligonucleotide decoy comprises a double-stranded sequence represented by formula (32):

(32)
5′-S₁n₂n₃C₄C₅T₆W₇T₈G₉C₁₀C₁₁T₁₂y₁₃y₁₄y₁₅y₁₆y₁₇n₁₈n₁₉
n₂₀ . . . . . . y₂₁y₂₂y₂₃y₂₄y₂₅G₂₆C₂₇C₂₈T₂₉C₃₀C₃₁
T₃₂W₃₃S₃₄n₃₅n₃₆S₃₇-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “W” can be A or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (32) has at least about 50%, 55%, 60%, 65%70%, 75%, 80%, 85%, 88%, 90%, 91%, 92%, 93%, 94%, 95%, 96%, 97%, 98%, or 99% sequence identity to the nucleotide sequence of SEQ ID NO.: 32. Such oligonucleotide decoys can bind to KLF4 transcription factor. In certain embodiments, such oligonucleotide decoys can bind to one or more transcription factors closely related to KLF4 transcription factor, such as KLF-1, -5.

In certain embodiments, an oligonucleotide decoy represented by formula (32) comprises a deletion of one or more (e.g., 1, 2, 3, 4, 5, 6, 7, 8, 9, 10, 11, 12 or 13) nucleotides selected from the group consisting of y₁₃, y₁₄, y₁₅, y₁₆, y₁₇, n₁₈, n₁₉, n₂₀, y₂₁, y₂₂, y₂₃, y₂₄ and y₂₅. In certain embodiments, oligonucleotide decoys comprising a deletion of one or more nucleotides selected from the group consisting of y₁₃, y₁₄, y₁₅, y₁₆, y₁₇, n₁₈, n₁₉, n₂₀, y₂₁, y₂₂, y₂₃, y₂₄ and y₂₅ have at least 50% identity to the nucleotide sequence of SEQ ID NO.: 32.

In certain embodiments, an oligonucleotide decoy comprises a double-stranded sequence represented by formula (33):

(33)
5′-S₁n₂n₃n₄W₅W₆W₇G₈G₉G₁₀w₁₁d₁₂g₁₃n₁₄n₁₅w₁₆w₁₇w₁₈G₁₉G₂₀. . .
. . . G₂₁W₂₂D₂₃G₂₄n₂₅n₂₆n₂₇n₂₈S₂₉-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, “D” can be an A, G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (33) has at least about 75%, 80%, 85%, 90%, 91%, 92%, 93%, 94%, 95%, 96%, 97%, 98%, or 99% sequence identity to the nucleotide sequence of SEQ ID NO.: 33. Such oligonucleotide decoys can bind to KLF7 transcription factor. In certain embodiments, such oligonucleotide decoys can bind to one or more transcription factors closely related to KLF7 transcription factor, such as KLF-1, -2, and -5.

In certain embodiments, an oligonucleotide decoy represented by formula (33) comprises a deletion of one or more (e.g., 1, 2, 3, 4, 5, 6, 7 or 8) nucleotides selected from the group consisting of w₁₁, d₁₂, g₁₃, n₁₄, n₁₅, w₁₆, w₁₇ and w₁₈. In certain embodiments, oligonucleotide decoys comprising a deletion of one or more nucleotides selected from the group consisting of w₁₁, d₁₂, g₁₃, n₁₄, n₁₅, w₁₆, w₁₇ and w₁₈ have at least 75% identity to the nucleotide sequence of SEQ ID NO.: 33.

In certain embodiments, an oligonucleotide decoy comprises a double-stranded sequence represented by formula (34):

(34)
5′-S₁w₂w₃w₄w₅w₆C₇A₈C₉T₁₀C₁₁A₁₂G₁₃C₁₄w₁₅w₁₆w₁₇w₁₈C₁₉g₂₀ . . .
. . . g₂₁w₂₂g₂₃w₂₄G₂₅G₂₆G₂₇W₂₈W₂₉g₃₀w₃₁w₃₂w₃₃w₃₄w₃₅S₃₆-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (34) has at least about 55%, 60%, 65%, 70%, 75%, 80%, 85%, 90%, 91%, 92%, 93%, 94%, 95%, 96%, 97%, 98%, or 99% sequence identity to the nucleotide sequence of SEQ ID NO.: 34. Such oligonucleotide decoys can bind to MAFG transcription factor. In certain embodiments, such oligonucleotide decoys can bind to one or more transcription factors closely related to MAFG transcription factor, such as MAF-A, -B, -F, -K.

In certain embodiments, an oligonucleotide decoy represented by formula (34) comprises a deletion of one or more (e.g., 1, 2, 3, 4, 5, 6, 7, 8, 9 or 10) nucleotides selected from the group consisting of w₁₅, w₁₆, w₁₇, w₁₈, c₁₉, g₂₀, g₂₁, w₂₂, g₂₃ and w₂₄. In certain embodiments, oligonucleotide decoys comprising a deletion of one or more nucleotides selected from the group consisting of w₁₅, w₁₆, w₁₇, w₁₈, c₁₉, g₂₀, g₂₁, w₂₂, g₂₃ and w₂₄ have at least 55% identity to the nucleotide sequence of SEQ ID NO.: 34.

In certain embodiments, an oligonucleotide decoy comprises a double-stranded sequence represented by formula (35):

(35)
5′-S₁n₂n₃W₄B₅Y₆A₇G₈Y₉A₁₀C₁₁C₁₂D₁₃
N₁₄R₁₅G₁₆H₁₇S₁₈A₁₉G₂₀ . . .
. . . C₂₁N₂₂N₂₃H₂₄n₂₅n₂₆n₂₇W₂₈B₂₉Y₃₀
A₃₁G₃₂Y₃₃A₃₄C₃₅C₃₆D₃₇N₃₈R₃₉G₄₀ . . .
. . . H₄₁S₄₂A₄₃G₄₄C₄₅N₄₆N₄₇H₄₈n₄₉n₅₀S₅₁-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, Y can be a C or a T, “H” can be an A, T or a C, “R” can be G or A, “D” can be G, A or T, “Y” can be C or T, “B” can be C, G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (35) has at least about 50%, 55%, 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 35. Such oligonucleotide decoys can bind to REST transcription factor.

In certain embodiments, an oligonucleotide decoy represented by formula (35) comprises a deletion of one or more (e.g., 1, 2 or 3) nucleotides selected from the group consisting of n₂₅, n₂₆ and n₂₇. In certain embodiments, oligonucleotide decoys comprising a deletion of one or more nucleotides selected from the group consisting of n₂₅, n₂₆ and n₂₇ have at least 50% identity to the nucleotide sequence of SEQ ID NO.: 35.

In certain embodiments, an oligonucleotide decoy comprises a double-stranded sequence represented by formula (36):

(36)
5′-S₁n₂n₃n₄n₅G6A₇R₈M₉A₁₀W₁₁k₁₂s₁₃a₁₄g₁₅k₁₆n₁₇n₁₈n₁₉n₂₀ . . .
. . . g₂₁a₂₂r₂₃M₂₄A₂₅W₂₆K₂₇S₂₈A₂₉G₃₀K₃₁n₃₂n₃₃n₃₄n₃₅S₃₆-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, “M” can be A or C, “R” can be A or G, “K” can be G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (36) has at least about 60%, 65%, 70%, 75%, 80%, 85%, 90%, 91%, 92%, 93%, 94%, 95%, 96%, 97%, 98%, or 99% sequence identity to the nucleotide sequence of SEQ ID NO.: 36. Such oligonucleotide decoys can bind to KCNIP3 transcription factor.

In certain embodiments, an oligonucleotide decoy represented by formula (36) comprises a deletion of one or more (e.g., 1, 2, 3, 4, 5, 6, 7, 8, 9, 10, 11, 12 or 13) nucleotides selected from the group consisting of k₁₂, s₁₃, a₁₄, g₁₅, k₁₆, n₁₇, n₁₈, n₁₉, n₂₀, g₂₁, a₂₂, r₂₃ and m₂₄. In certain embodiments, oligonucleotide decoys comprising a deletion of one or more nucleotides selected from the group consisting of k₁₂, s₁₃, a₁₄, g₁₅, k₁₆, n₁₇, n₁₈, n₁₉, n₂₀, g₂₁, a₂₂, r₂₃ and m₂₄ have at least 60% identity to the nucleotide sequence of SEQ ID NO.: 36.

In certain embodiments, an oligonucleotide decoy comprises a double-stranded sequence represented by formula (37):

(37)
5′-S₁n₂n₃n₄n₅G₆A₇R₈G₉C₁₀C₁₁S₁₂S₁₃w₁₄g₁₅w₁₆n₁₇n₁₈n₁₉n₂₀ . . .
. . . g₂₁a₂₂r₂₃G₂₄C₂₅C₂₆S₂₇S₂₈W₂₉G₃₀W₃₁n₃₂n₃₃n₃₄S₃₅-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, “M” can be A or C, “R” can be A or G, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (37) has at least about 75%, 80%, 85%, 90%, 91%, 92%, 93%, 94%, 95%, 96%, 97%, 98%, or 99% sequence identity to the nucleotide sequence of SEQ ID NO.: 37. Such oligonucleotide decoys can bind to KCNIP3 transcription factor.

In certain embodiments, an oligonucleotide decoy represented by formula (37) comprises a deletion of one or more (e.g., 1, 2, 3, 4, 5, 6, 7, 8, 9, 10 or 11) nucleotides selected from the group consisting of s₁₃, w₁₄, g₁₅, w₁₆, n₁₇, n₁₈, n₁₉, n₂₀, g₂₁, a₂₂ and r₂₃. In certain embodiments, oligonucleotide decoys comprising a deletion of one or more nucleotides selected from the group consisting of s₁₃, w₁₄, g₁₅, w₁₆, n₁₇, n₁₈, n₁₉, n₂₀, g₂₁, a₂₂ and r₂₃ have at least 75% identity to the nucleotide sequence of SEQ ID NO.: 37.

In certain embodiments, an oligonucleotide decoy comprises a double-stranded sequence represented by formula (38):

(38)
5′-s₁C₂G₃A₄A₅A₆G₇G₈A₉C₁₀A₁₁A₁₂
A₁₃s₁₄s₁₅n₁₆v₁₇v₁₈n₁₉n₂₀ . . .
. . . n₂₁S₂₂g₂₃d₂₄n₂₅n₂₆G₂₇G₂₈A₂₉
C₃₀A₃₁A₃₂A₃₃G₃₄G₃₅T₃₆C₃₇A₃₈S₃₉-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “V” can be A, C or G, “D” can be G, A or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (38) has at least about 50%, 55%, 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 38. Such oligonucleotide decoys can bind to PPARA transcription factor. In certain embodiments, such oligonucleotide decoys can bind to one or more transcription factors closely related to PPARA transcription factor, such as PPAR-D, -G.

In certain embodiments, an oligonucleotide decoy represented by formula (38) comprises a deletion of one or more (e.g., 1, 2, 3, 4, 5, 6, 7, 8, 9 or 10) nucleotides selected from the group consisting of s₁₄, s₁₅, n₁₆, v₁₇, v₁₈, n₁₉, n₂₀, n₂₁, s₂₂ and g₂₃. In certain embodiments, oligonucleotide decoys comprising a deletion of one or more nucleotides selected from the group consisting of s₁₄, s₁₅, n₁₆, v₁₇, v₁₈, n₁₉, n₂₀, n₂₁, s₂₂ and g₂₃ have at least 50% identity to the nucleotide sequence of SEQ ID NO.: 38.

In certain embodiments, an oligonucleotide decoy comprises a double-stranded sequence represented by formula (39):

(39)
5′-S₁n₂n₃n₄A₅R₆M₇R₈W₉W₁₀y₁₁W₁₂M₁₃g₁₄n₁₅n₁₆a₁₇r₁₈m₁₉r₂₀ . . .
. . . w₂₁w₂₂y₂₃W₂₄M₂₅G₂₆A₂₇A₂₈T₂₉T₃₀n₃₁n₃₂n₃₃n₃₄S₃₅-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, “R” can be A or G, “M” can be an A or a C, “Y” can be a C or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (39) has at least about 55%, 60%, 65%, 70%, 75%, 80%, 85%, 90%, 91%, 92%, 93%, 94%, 95%, 96%, 97%, 98%, or 99% sequence identity to the nucleotide sequence of SEQ ID NO.: 39. Such oligonucleotide decoys can bind to HSF1 transcription factor. In certain embodiments, the oligonucleotide decoys can bind to one or more transcription factors closely related to HSF1 transcription factor, such as HSF2.

In certain embodiments, an oligonucleotide decoy represented by formula (39) comprises a deletion of one or more (e.g., 1, 2, 3, 4, 5, 6, 7, 8, 9, 10, 11, 12 or 13) nucleotides selected from the group consisting of y₁₁, w₁₂, m₁₃, g₁₄, n₁₅, n₁₆, a₁₇, r₁₈, m₁₉, r₂₀, w₂₁, w₂₂ and y₂₃. In certain embodiments, oligonucleotide decoys comprising a deletion of one or more nucleotides selected from the group consisting of y₁₁, w₁₂, m₁₃, g₁₄, n₁₅, n₁₆, a₁₇, r₁₈, m₁₉, r₂₀, w₂₁, w₂₂ and y₂₃ have at least 55% identity to the nucleotide sequence of SEQ ID NO.: 39.

In certain embodiments, an oligonucleotide decoy comprises a double-stranded sequence represented by formula (47):

(47)
5′-S₁n₂n₃n₄n₅n₆C₇A₈C₉T₁₀T₁₁C₁₂
C₁₃T₁₄G₁₅C₁₆n₁₇n₁₈n₁₉n₂₀n₂₁S₂₂-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (47) has at least about 80%, 85%, 88%, 90%, 91%, 92%, 93%, 94%, 95%, 96%, 97%, 98%, or 99% sequence identity to the nucleotide sequence of SEQ ID NO.: 47. Such oligonucleotide decoys can bind to ELK1 transcription factor. In certain embodiments, such oligonucleotide decoys can bind to one or more transcription factors closely related to ELK1 transcription factor, such as ETS1.

In certain embodiments, an oligonucleotide decoy represented by formula (47) comprises a deletion of one or more (e.g., 1, 2, 3, 4, 5, 6, 7, 8, 9 or 10) nucleotides selected from the group consisting of n₂, n₃, n₄, n₅, n₆, n₁₇, n₁₈, n₁₉, n₂₀ and n₂₁. In certain embodiments, oligonucleotide decoys comprising a deletion of one or more nucleotides selected from the group consisting of n₂, n₃, n₄, n₅, n₆, n₁₇, n₁₈, n₁₉, n₂₀ and n₂₁ have at least 80% identity to the nucleotide sequence of SEQ ID NO.: 47.

In certain embodiments, an oligonucleotide decoy comprises a double-stranded sequence represented by formula (48):

(48)
5′-S₁n₂n₃n₄n₅n₆A₇G₈K₉Y₁₀A₁₁A₁₂D₁₃N₁₄D₁₅T₁₆W₁₇N₁₈M₁₉N₂₀ . . .
. . . n21n22n23n24n25S26-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “V” can be G, A or C, “K” can be T or G, “D” can be G, A or T, “W” can be A or T, “M” can be C or A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (48) has at least about 70%, 75%, 80%, 85%, 88%, 90%, 91%, 92%, 93%, 94%, 95%, 96%, 97%, 98%, or 99% sequence identity to the nucleotide sequence of SEQ ID NO.: 48. Such oligonucleotide decoys can bind to HNF1A transcription factor. In certain embodiments, such oligonucleotide decoys can bind to one or more transcription factors closely related to HNF1A transcription factor, such as HNF1B-C.

In certain embodiments, an oligonucleotide decoy represented by formula (48) comprises a deletion of one or more (e.g., 1, 2, 3, 4, 5, 6, 7, 8, 9 or 10) nucleotides selected from the group consisting of n₂, n₃, n₄, n₅, n₆, n₂₁, n₂₂, n₂₃, n₂₄ and n₂₅. In certain embodiments, oligonucleotide decoys comprising a deletion of one or more nucleotides selected from the group consisting of n₂, n₃, n₄, n₅, n₆, n₂₁, n₂₂, n₂₃, n₂₄ and 1125 have at least 70% identity to the nucleotide sequence of SEQ ID NO.: 48.

In certain embodiments, an oligonucleotide decoy comprises a double-stranded sequence represented by formula (49):

(49)
5′-S₁n₂n₃T₄C₅T₆C₇Y₈G₉A₁₀T₁₁T₁₂G₁₃G₁₄Y₁₅T₁₆C₁₇B₁₈Y₁₉n₂₀S₂₁-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “B” can be C, G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (49) has at least about 80%, 85%, 88%, 90%, 91%, 92%, 93%, 94%, 95%, 96%, 97%, 98%, or 99% sequence identity to the nucleotide sequence of SEQ ID NO.: 49. Such oligonucleotide decoys can bind to NFYA transcription factor. In certain embodiments, such oligonucleotide decoys can bind to one or more transcription factors closely related to NFYA transcription factor, such as NFYB-C.

In certain embodiments, an oligonucleotide decoy represented by formula (49) comprises a deletion of one or more (e.g., 1, 2 or 3) nucleotides selected from the group consisting of n₂, n₃ and n₂₀. In certain embodiments, oligonucleotide decoys comprising a deletion of one or more nucleotides selected from the group consisting of n₂, n₃ and n₂₀ have at least 80% identity to the nucleotide sequence of SEQ ID NO.: 49.

In certain embodiments, an oligonucleotide decoy comprises a double-stranded sequence represented by formula (50):

(50)
5′-S₁n₂n₃n₄n₅n₆C₇C₈T₉W₁₀T₁₁G₁₂C₁₃C₁₄T₁₅C₁₆C₁₇T₁₈W₁₉S₂₀ . . .
. . . r₂₁r₂₂n₂₃n₂₄n₂₅S₂₆-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be A or T, “R” can be G or A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (50) has at least about 75%, 80%, 85%, 88%, 90%, 91%, 92%, 93%, 94%, 95%, 96%, 97%, 98%, or 99% sequence identity to the nucleotide sequence of SEQ ID NO.: 50. Such oligonucleotide decoys can bind to KLF4 transcription factor. In certain embodiments, such oligonucleotide decoys can bind to one or more transcription factors closely related to KLF4 transcription factor, such as KLF-1, -5.

In certain embodiments, an oligonucleotide decoy represented by formula (50) comprises a deletion of one or more (e.g., 1, 2, 3, 4, 5, 6, 7, 8, 9 or 10) nucleotides selected from the group consisting of n₂, n₃, n₄, n₅, n₆, r₂₁, r₂₂, n₂₃, n₂₄ and n₂₅. In certain embodiments, oligonucleotide decoys comprising a deletion of one or more nucleotides selected from the group consisting of n₂, n₃, n₄, n₅, n₆, r₂₁, r₂₂, n₂₃, n₂₄ and n₂₅ have at least 75% identity to the nucleotide sequence of SEQ ID NO.: 50.

In certain embodiments, an oligonucleotide decoy comprises a double-stranded sequence represented by formula (51):

(51)
5′-S₁n₂n₃n₄n₅W₆B₇Y₈A₉G₁₀Y₁₁A₁₂C₁₃C₁₄D₁₅N₁₆R₁₇G₁₈H₁₉S₂₀ . . .
..A₂₁G₂₂C₂₃N₂₄N₂₅H₂₆n₂₇n₂₈n₂₉n₃₀S₃₁-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, “H” can be an A, T or a C, “R” can be G or A, “D” can be G, A or T, “Y” can be C or T, “B” can be C, G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (51) has at least about 75%, 80%, 85%, 88%, 90%, 91%, 92%, 93%, 94%, 95%, 96%, 97%, 98%, or 99% sequence identity to the nucleotide sequence of SEQ ID NO.: 51. Such oligonucleotide decoys can bind to REST transcription factor.

In certain embodiments, an oligonucleotide decoy represented by formula (51) comprises a deletion of one or more (e.g., 1, 2, 3, 4, 5, 6, 7 or 8) nucleotides selected from the group consisting of n₂, n₃, n₄, n₅, n₂₇, n₂₈, n₂₉ and n₃₀. In certain embodiments, oligonucleotide decoys comprising a deletion of one or more nucleotides selected from the group consisting of n₂, n₃, n₄, n₅, n₂₇, n₂₈, n₂₉ and n₃₀ have at least 75% identity to the nucleotide sequence of SEQ ID NO.: 51.

In certain embodiments, an oligonucleotide decoy comprises a double-stranded sequence represented by formula (52):

(52)
5′-S₁m₂r₃m₄W₅A₆G₇G₈N₉C₁₀A₁₁A₁₂A₁₃G₁₄G₁₅T₁₆C₁₇A₁₈n₁₉n₂₀ . . .
. . . n₂₁n₂₂S₂₃-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be A or T, “R” can be G or A, “M” can be C or A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (52) has at least about 80%, 85%, 88%, 90%, 91%, 92%, 93%, 94%, 95%, 96%, 97%, 98%, or 99% sequence identity to the nucleotide sequence of SEQ ID NO.: 52. Such oligonucleotide decoys can bind to PPARA transcription factor. In certain embodiments, such oligonucleotide decoys can bind to one or more transcription factors closely related to PPARA transcription factor, such as PPAR-D, -G.

In certain embodiments, an oligonucleotide decoy represented by formula (52) comprises a deletion of one or more (e.g., 1, 2, 3, 4, 5, 6, 7 or 8) nucleotides selected from the group consisting of m₂, r₃, m₄, n₁₉, n₂₀, n₂₁, n₂₂ and g₂₃. In certain embodiments, oligonucleotide decoys comprising a deletion of one or more nucleotides selected from the group consisting of m₂, r₃, m₄, n₁₉, n₂₀, n₂₁, n₂₂ and g₂₃ₕ have at least 80% identity to the nucleotide sequence of SEQ ID NO.: 52.

In certain embodiments, an oligonucleotide decoy comprises a double-stranded sequence represented by formula (53):

(53)
5′-S₁s₂c₃t₄t₅g₆y₇k₈g₉y₁₀k₁₁G₁₂A₁₃A₁₄T₁₅A₁₆T₁₇c₁₈g₁₉n₂₀ . . .
. . . n₂₁n₂₂n₂₃n₂₄S₂₅-3′

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “K” can be T or G, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (53) has at least about 75%, 80%, 85%, 88%, 90%, 91%, 92%, 93%, 94%, 95%, 96%, 97%, 98%, or 99% sequence identity to the nucleotide sequence of SEQ ID NO.: 53. Such oligonucleotide decoys can bind to TEAD1 transcription factor. In certain embodiments, such oligonucleotide decoys can bind to one or more transcription factors closely related to TEAD1 transcription factor, such as TEAD2-4.

In certain embodiments, an oligonucleotide decoy represented by formula (53) comprises a deletion of one or more (e.g., 1, 2, 3, 4, 5, 6, 7, 8, 9, 10, 11, 12, 13, 14, 15, 16 or 17) nucleotides selected from the group consisting of s₂, c₃, t₄, t₅, g₆, y₇, k₈, g₉, y₁₀, k₁₁, c₁₈, g₁₉, n₂₀, n₂₁, n₂₂, n₂₃ and n₂₄. In certain embodiments, oligonucleotide decoys comprising a deletion of one or more nucleotides selected from the group consisting of s₂, c₃, t₄, t₅, g₆, y₇, k₈, g₉, y₁₀, k₁₁, c₁₈, g₁₉, n₂₀, n₂₁, n₂₂, n₂₃ and n₂₄ have at least 75% identity to the nucleotide sequence of SEQ ID NO.: 53.

A double stranded oligonucleotide having a certain percent (e.g., 65%, 70%, 75%, 80%, 85%, 90%, 95%, or 99%) of sequence identity with another sequence means that, when aligned, that percentage determines the level of correspondence of bases arrangement in comparing the two sequences. This alignment and the percent homology or identity can be determined using any suitable software program known in the art that allows local alignment. The software program should be capable of finding regions of local identity between two sequences without the need to include the entire length of the sequences. In some embodiments, such program includes but is not limited to the EMBOSS Pairwise Alignment Algorithm (available from the European Bioinformatics Institute (EBI)), the ClustalW program (also available from the European Bioinformatics Institute (EBI)), or the BLAST program (BLAST Manual, Altschul et al., Natl Cent. Biotechnol. Inf., Natl Lib. Med. (NCIB NLM NIH), Bethesda, Md., and Altschul et al., (1997) NAR 25:3389 3402).

One skilled in the art will recognize that sequences encompassed herein include those that hybridize under stringent hybridization conditions with an exemplified sequence (e.g., SEQ ID NOs.: 1-42, 45, and 47-53). A nucleic acid is hybridizable to another nucleic acid when a single stranded form of the nucleic acid can anneal to the other single stranded nucleic acid under appropriate conditions of temperature and solution ionic strength. Hybridization conditions are well known in the art. In some embodiments, annealing can occur during a slow decrease of temperature from a denaturizing temperature (e.g., 100° C.) to room temperature in a salt containing solvent (e.g., Tris-EDTA buffer).

Generally, the oligonucleotide decoys disclosed herein may be used to bind and, e.g., thereby inhibit, transcription factors that modulate the expression of genes involved with nociceptive signaling and/or a subject's (e.g., patient's) perception of pain. A oligonucleotide decoy disclosed herein designed to bind to a specific transcription factor has a nucleic acid sequence mimicking the endogenous genomic DNA sequence normally bound by the transcription factor. Accordingly, the oligonucleotide decoys disclosed herein inhibit a necessary step for gene expression. Further, the oligonucleotide decoys disclosed herein may bind to a number of different transcription factors.

### Chemically Modified Oligonucleotide Decoys

The oligonucleotide decoys disclosed herein can be chemically modified by methods well known to the skilled artisan (e.g., incorporation of phosphorothioate, methylphosphonate, phosphorodithioate, phosphoramidates, carbonate, thioether, siloxane, acetamidate or carboxymethyl ester linkages between nucleotides) to prevent degradation by nucleases within cells and extra-cellular fluids (e.g., serum, cerebrospinal fluid). Also, oligonucleotide decoys can be designed that form hairpin and dumbbell structures which also prevent or hinder nuclease degradation. Further, the oligonucleotide decoys can also be inserted as a portion of a larger plasmid capable of episomal maintenance or constitutive replication in the target cell in order to provide longer term, enhanced intracellular exposure to the decoy sequence or reduce its degradation. Accordingly, any chemical modification or structural alteration known in the art to enhance oligonucleotide stability is within the scope of the present disclosure. In some embodiments, the oligonucleotide decoys disclosed herein can be attached, for example, to polyethylene glycol polymers, peptides (e.g., a protein translocation domain) or proteins which improve the therapeutic effect of oligonucleotide decoys. Such modified oligonucleotide decoys can preferentially traverse the cell membrane.

In certain embodiments, the oligonucleotide decoys are provided as salts, hydrates, solvates, or N-oxide derivatives. In certain embodiments, the oligonucleotide decoys are provided in solution (e.g., a saline solution having a physiologic pH) or in lyophilized form. In other embodiments, the oligonucleotide decoys are provided in liposomes.

### Kits

In certain embodiments, one or more oligonucleotide inhibitors (e.g., oligonucleotide decoys) are provided in a kit. In certain embodiments, the kit includes an instruction, e.g., for using said one or more oligonucleotide inhibitors. In certain embodiments, said instruction describes one or more of the methods of the present invention, e.g., a method for preventing or treating pain in a high PCS score patients. In certain embodiments, the oligonucleotide inhibitors provided in a kit are provided in lyophilized form. In certain related embodiments, a kit that comprises one or more lyophilized oligonucleotide inhibitors further comprises a solution (e.g., a pharmaceutically acceptable saline solution) that can be used to resuspend said one or more of the oligonucleotide inhibitors.

In certain embodiments, oligonucleotide inhibitors include, but are not limited to, oligonucleotide decoys comprising sequences presented in Table A. In general, the oligonucleotide decoy is generated by annealing the sequence provided in the table with a complementary sequence. To generate a mismatch double-stranded oligonucleotide, the sequence provided in the table can be annealed to a sequence that is only partially complementary. For example, SEQ ID NO.:43 can be annealed to SEQ ID NO.:46 to produce the mismatched sequence, SEQ ID NO.:43/46.

TABLE A

| Oligonucleotide | |
|---|---|
| Sequences | |
| (5′-3′) | SEQ ID NO. |
| GGCTTATGCAAATTCG | SEQ ID NO.: 1 |
| AATGCAAATTTGTCG | |
| CTAAGCCCACGTGACC | SEQ ID NO.: 2 |
| ATTGGCCAGGTGACCA | |
| GATC | |
| GTTATGCGTGGGCGAT | SEQ ID NO.: 3 |
| AATGCGGGGGCGTTAT | |
| AG | |
| GCCTCCCTGAGCTCAT | SEQ ID NO.: 4 |
| TGACGTATCTCGG | |
| CGAATATGACTGAGAA | SEQ ID NO.: 5 |
| TGACTCAGATTTGC | |
| GGTTCTATGATTTTGG | SEQ ID NO.: 6 |
| AATCGGATTGTGCAAA | |
| GAAGC | |
| GCTTCAGGATGTCCAT | SEQ ID NO.: 7 |
| ATTAGGAGATCTTGTT | |
| CG | |
| GGCCACAGGATGTAGGAT | SEQ ID NO.: 8 |
| GTCCATATTAGGATGC | |
| GTTCTCTAAAAATAAAAG | SEQ ID NO.: 9 |
| GCTAAAAATAAAAGTCG | |
| ATTAGGGGCGGGGTCCGG | SEQ ID NO.: 10 |
| GGCGGGGTATTA | |
| GTTATGGCGGGGCGGGGC | SEQ ID NO.: 11 |
| GGGGCCGGGCGGTTTAC | |
| GGCAATGTGGTTTTAGTG | SEQ ID NO.: 12 |
| TGGTTTTACGG | |
| GCCGTTTGGGGTCATAGA | SEQ ID NO.: 13 |
| ACCACAGGAACCACACGG | |
| CATTGCCCGGAAATGGA | SEQ ID NO.: 14 |
| CCGGATGTAATTTCC | |
| GTTCTTGGAAAATAAATG | SEQ ID NO.: 15 |
| GAAAATAGTGGAAAATAAG | |
| TCG | |
| CGTTCCCACTTCCTGCGA | SEQ ID NO.: 16 |
| CCACTTCCTGCCGGG | |
| CTGCACCTATAAATGGC | SEQ ID NO.: 17 |
| CTATAAATGGGGATGC | |
| GCTTATTTCGCGGAAGG | SEQ ID NO.: 18 |
| TTTCCCGGAAGTGGCG | |
| GCTGTGCCTTATCTCTT | SEQ ID NO.: 19 |
| TGGGATAACTGGCG | |
| GCTTAATGAATAAGAGG | SEQ ID NO.: 20 |
| AAAAATGCATGCTGG | |
| GTTCTGAGATTGCACGA | SEQ ID NO.: 21 |
| TGAGATTTCACAGTCG | |
| GTCCCGCATAAATAATG | SEQ ID NO.: 22 |
| GCATCCTTAATCGCG | |
| GTGCAGGCAAGAGTAGAG | SEQ ID NO.: 23 |
| ACAGGCAAGAGTAGATGC | |
| CCGCCAATAATTAATT | SEQ ID NO.: 24 |
| ATTAAGGCC | |
| GCTTCGTTCCATTTCCGG | SEQ ID NO.: 25 |
| TCTCGGTTTCCCCATTC | |
| GCTGCTGTGGAATATCG | SEQ ID NO.: 26 |
| ACCTGTGGAATATCGTG | |
| GCCGTATAAATGTGCTATA | SEQ ID NO.: 27 |
| AAAGTTTTAAGACCGTGC | |
| GCCGTATAAATGTGCTAT | SEQ ID NO.: 28 |
| AAAAGCCGTGC | |
| ATGCTGCGCTTTTCTCC | SEQ ID NO.: 29 |
| AATCTGCGG | |
| CGTTCTCCGATTGGTCA | SEQ ID NO.: 30 |
| CGGACTCTCCGATTGGT | |
| CACGGC | |
| GCGCACCCCAGCCTGGC | SEQ ID NO.: 31 |
| TCACCCACGCG | |
| GATCCTTTGCCTCCTTCGA | SEQ ID NO.: 32 |
| TCCTTTGCCTCCTTCAAG | |
| GGTGTTTGGGAGAGCTT | SEQ ID NO.: 33 |
| TGGGAGGATACG | |
| GCTAATCACTCAGCATTT | SEQ ID NO.: 34 |
| CGGTGAGGGAAGTGAAAG | |
| CCTTTCAGCACCACGGA | SEQ ID NO.: 35 |
| CAGCGCCAGCTTCAGCA | |
| CCACGGACAGCGCCTCG | |
| GGATCGAACATGGAGTCA | SEQ ID NO.: 36 |
| GTGAGAAATCAGGATCGG | |
| GGATCGAAGCCGGAGTC | SEQ ID NO.: 37 |
| AAGGAGGCCCCTGATCGG | |
| CCGAAAGGACAAAGGTC | SEQ ID NO.: 38 |
| AAGTCGAAAGGACAAAG | |
| GTCAG | |
| CGGGAGAAAATTCGGGA | SEQ ID NO.: 39 |
| ACGTTCAAGAATTGTCGG | |
| GTTATGCGTGGGCGTAG | SEQ ID NO.: 40 |
| ATGCGGGGGCGTTATAG | |
| GATGCGTGGGCGTAGG | SEQ ID NO.: 41 |
| GTATGCGTGGGCGGTGG | SEQ ID NO.: 42 |
| GCGTAG | |
| GTTATGCGTTTGTAGAT | SEQ ID NO.: 43 |
| GCTTTCGTTATAG | |
| GTTATGCGTGGGCGATA | SEQ ID NO.: 44 |
| TAG | |
| GATGCGTGGGCGTTGAC | SEQ ID NO.: 45 |
| GTGGAAAATGC | |
| CTATTTCGAAACGATCT | SEQ ID NO.: 46 |
| ACATTGGCATAAC | |
| CGTTCCCACTTCCTGC | SEQ ID NO.: 47 |
| GACCGG | |
| GGGTGAAGGCAAGAGT | SEQ ID NO.: 48 |
| AGAGCGGCGG | |
| CGTTCTCCGATTGGTCA | SEQ ID NO.: 49 |
| CGCG | |
| GTACTCCCTTTGCCTCC | SEQ ID NO.: 50 |
| TTCAACCGG | |
| CCTTATTCAGCACCAC | SEQ ID NO.: 51 |
| GGACAGCGCCATTCG | |
| GCGAAAGGACAAAGGT | SEQ ID NO.: 52 |
| CAGGCGG | |
| GGCTTGCTGTGGAATA | SEQ ID NO.: 53 |
| TCGATGGTG | |

Reference will now be made in detail to particular embodiments of the disclosure found in the Examples. These Examples are not intended to limit the disclosure to those particular embodiments. To the contrary, the disclosure is intended to cover alternatives, modifications, and equivalents, as may be included within the spirit and scope of the invention, as defined by the appended claims.

### EXAMPLES

### Example 1: ADYX-004 Clinical Trial

The ADYX-004 trial was a phase 2 randomized double-blinded placebo controlled study to evaluate the safety and efficacy of a single intrathecal preoperative administration of AYX1, an oligonucleotide decoy, in patients undergoing unilateral total knee arthroplasty. AYX1 is also known as brivoligide (generic name) and comprises the sequence of SEQ ID NO. 42 (5′-GTATGCGTGGGCGGTGGGCGTAG-3′) as a sense strand and the antisense strand having the sequence of 3′-CATACGCACCCGCCACCCGCATC-5′.

Methods Overview

Subjects enrolled in the study were randomized in a 1:1 ratio to AYXI Injection (AYX1 Injection 660 mg/6 mL) or placebo (Placebo 6 mL), with randomization stratified by baseline Pain Catastrophizing Scale (PCS) score (≥20/<20). AYX1 Injection was administered intrathecally before surgery in patients undergoing primary unilateral total knee arthroplasty.

Results Overview

Pre-specified efficacy endpoints of AYX1 in the total study population in ADYX-004 were not supported by data but AYX1 treatment effect was evident in the subpopulation that scored high on the PCS.

When the ADYX-004 results were filtered by PCS ≥20 and PCS ≥16, AYX1 showed a substantial treatment effect across multiple endpoints:
Approximately 25% to 35% reduction in pain at rest
Approximately 20% to 30% reduction in pain with walking
Approximately 18% to 20% reduction in worst pain
Approximately 20 to 26 days reduction in time to achieve NRS ≤3 for worst pain
Approximately 35% to 40% reduction in opioid consumption

In summary, AYX1 demonstrated clinically meaningful benefits in the subjects who score high on the PCS, a difficult-to-treat population with higher risk of increased pain and opioid use. In light of the literature and the current knowledge in the field and the initial rationale for stratifying the trial by PCS, this is considered an unexpected finding.

### Detailed Study

Methodology

Patients providing informed consent and meeting all study eligibility criteria were enrolled in the study on the day of surgery (Day 1) and randomized to receive either intrathecal AYX1 Injection or intrathecal placebo. A screening visit was conducted within 21 days of randomization, and included assessment of baseline pain (pain with rising from a seated position, and worst pain, least pain, and average pain over the last 24 hours, as well as pain at rest and with a 15 meter walk).

Subjects randomized to the AYX1 treatment group received a single 660 mg/6 mL intrathecal administration of AYX1 Injection as a slow bolus injection just prior to administration of spinal anesthesia, via the same needle. Subjects randomized to the placebo group received a single 6 mL intrathecal injection of placebo (vehicle control) as a slow bolus injection just prior to administration of spinal anesthesia, via the same needle. Subjects remained seated for at least two minutes after the start of the injection and then placed supine for surgery. Subjects remained hospitalized for at least 48 hours (to Day 3) after completion of surgery (close of incision); inpatient study assessments were conducted through 48 hours (Day 3).

All subjects enrolled in the study had a standardized set of analgesic options (described in the “Surgical Anesthesia/Sedation” and “Postoperative Analgesic Options” sections below). Standard local procedures were allowed for prophylactic antibiotics, venous thromboembolism (VTE) prophylaxis (i.e., anticoagulant use, compression stockings and boots), and anti-emetics.

All subjects enrolled in the study underwent standard physical therapy (PT) as indicated; the frequency of PT was documented.

Adverse events were recorded from the time of randomization and SAEs were recorded from the time of consent. Adverse events and SAEs were monitored until discharge from the hospital and will be recorded at the follow-up visits through Day 28. Physical examination findings and vital signs were recorded through Day 3, and laboratory assessments were recorded through Day 28. Concomitant medications were collected through Day 28; analgesic medications were collected through Day 90.

Pain at rest and with walking were recorded by study staff during the inpatient stay and at follow-up visits. If used, knee immobilizers, continuous passive motion (CPM), and cooling devices were required to be discontinued ˜30 minutes before study pain assessments. Daily ratings of pain with rising from a seated position, and worst pain, least pain, and average pain over the previous 24 hours were collected via eDiary by subjects every evening from Day 3 until the Day 90 visit. Analgesic medication use was collected via eDiary by subjects daily after discharge until the Day 90 visit. Follow-up (FU) visits occurred on Days 7, 14, 21, 28 (±2 days), and 42, 63 and 90 (±5 days).

Surgical Anesthesia/Sedation

Intraoperative anesthetic consisted of 10-17.5 mg bupivacaine administered in the lumbar intrathecal space following administration of study drug, via the same needle. Intravenous propofol was used for sedation. Intravenous midazolam and fentanyl may be used perioperatively.

General anesthesia or any use of a potent inhalational agent, peripheral nerve blocks, neuroaxial (intrathecal or epidural) opioids, preoperative extended release/long acting opioids, cryoneurolysis (including Iovera), ketamine, and systemic corticosteroids were not allowed.

A one-time perioperative infiltration of local anesthetic (including liposomal formulations) at the surgical wound site (which includes periarticular injections) was allowed. Steroids were not allowed to be included in the infiltration; other medications not excluded by the protocol may be included.

Acetaminophen and NSAIDs (including COX-2 inhibitors) were allowed.

All details of the anesthetic regimen were recorded.

Postoperative Analgesic Options

Postoperative analgesia was based on immediate release opioid therapy with all doses recorded. Following surgery, subjects were dosed to comfort; once pain was controlled, subjects could receive on demand opioids orally, intravenously, or via IV patient controlled analgesia (PCA) with demand bolus dosing only (no basal infusion rate). On the morning following surgery (or when indicated), IV PCA was discontinued (if used) and a PRN (as needed) oral opioid regimen was started. Subjects were encouraged to use the opioid medication only when needed for pain, rather than on a prescribed schedule. Extended release/long acting opioids (e.g., Oxycontin) were not allowed. Acetaminophen and NSAIDs (including COX-2 inhibitors) were allowed.

Other pain therapies: Cryoneurolysis (including lovera) on the current operative knee region, and ketamine were not allowed at any time through the duration of the study. Gabapentin (Neurontin) and pregabalin (Lyrica) were not allowed through Day 28. Use of systemic corticosteroids and/or intra-articular steroid injections were not allowed through Day 28.

Inclusion Criteria:

Subjects were required to meet ALL of the following inclusion criteria:

| | |
|---|---|
| 1. | Male or female, between 40-80 years of age, inclusive |
| 2. | Adequately informed of the nature and risks of the study and have given written |
| | informed consent before undergoing any study specific assessments or procedures |
| 3. | Scheduled to undergo primary unilateral TKA with spinal anesthesia for painful |
| | osteoarthritis without congenital knee pathology which would require more |
| | extensive bone surgery than normal |
| 4. | Have American Society of Anesthesiologists Physical Status Classification |
| | System ≤3 |
| 5. | Medically stable as determined by the Investigator based on pre-study medical |
| | history, physical examination, clinical laboratory tests, and 12-lead |
| | electrocardiogram (ECG) findings |
| 6. | Vital signs, clinical laboratory values, and prior/concomitant medication use |
| | acceptable for elective surgery with spinal anesthetic |
| 7. | Body weight ≥34 kg and body mass index of 18-40 kg/m² |
| 8. | Female subjects of child-bearing potential, and those <1 year post-menopausal, |
| | must have a negative serum pregnancy test at screening and agree to practice |
| | highly effective methods of birth control such as hormonal methods (e.g., |
| | combined oral, implantable, injectable, or transdermal contraceptives), other |
| | implantable methods (e.g., intrauterine device), double barrier methods (e.g., |
| | condoms, sponge, diaphragm, or vaginal ring plus spermicidal jellies or cream), or |
| | total abstinence from intercourse for 1 month after study drug administration |
| 9. | Male subjects who are sexually active must agree to use effective barrier |
| | contraception or remain abstinent for 1 month after study drug administration to |
| | prevent the transfer of seminal fluid |
| 10. | Have a stable medical regimen for ≥14 days before randomization (excluded |
| | medications are listed in Exclusion Criteria #9-12) |
| 11. | Able to read and understand study instructions in English, and willing and able to |
| | comply with all study procedures, including completing daily questionnaires via |
| | eDiary, returning for follow-up visits, and participating in standard physical |
| | therapy as indicated |

Exclusion Criteria:

Subjects must NOT meet any of the following exclusion criteria:

| | |
|---|---|
| 1. | Target knee has >20 degrees valgus or varus deformity (in the opinion of the |
| | Investigator; if there is any question, an x-ray must be obtained to confirm the |
| | severity of the deformity), evidence of significant bone loss or ligamentous laxity, |
| | or existing major hardware that requires removal during TKA |
| 2. | More than 2 other current focal areas of pain, none greater in intensity than the |
| | target knee and no other active chronic pain conditions that would compromise |
| | operative knee pain evaluation (e.g., CRPS, fibromyalgia) |
| 3. | Inflammatory arthridities (e.g., rheumatoid arthritis, lupus, ankylosing spondylitis, |
| | psoriatic arthritis), with the exception of clinically stable/non-active gout that does |
| | not affect the knee and does not interfere with walking |
| 4. | Undergoing concomitant surgical procedures (in addition to TKA) or non-elective |
| | TKA, or contralateral knee is likely to require TKA within 3 months (or would |
| | interfere with study assessments) |
| 5. | Operative arthroscopy in the surgical knee in the last 4 months (or in the |
| | contralateral knee in the last 2 months); meniscal repair in the surgical knee in the |
| | last 6 months (or in the contralateral knee in the last 3 months); other prior surgery |
| | in either knee in the last 9 months, except for diagnostic arthroscopy; or use of |
| | cryoneurolysis (including Iovera) on the current operative knee region within the 6 |
| | months prior to randomization and/or at any time through the duration of the study |
| 6. | Planned use of general anesthesia or potent inhalational agents, peripheral nerve |
| | block (e.g., femoral nerve block), neuroaxial (intrathecal or epidural) opioids, |
| | preoperative extended release/long acting opioids, or any use of ketamine |
| | preoperatively and/or at any time through the duration of the study |
| 7. | Known spinal deformities (congenital, degenerative, or due to surgery) that would |
| | interfere with standard intrathecal injections, or cutaneous infection in the lumbar |
| | area that would preclude intrathecal administration of study drug |
| 8. | Hospitalization or major surgery within 3 months of randomization |
| 9. | Use of more than 40 mg per day (on average) of oral morphine or its equivalent |
| | within 1 month prior to randomization |
| 10. | Use of gabapentin or pregabalin within 1 week prior to randomization or planned |
| | use post-operatively through Day 28 |
| 11. | Use of systemic corticosteroids (does not include inhaled steroids) within 3 months |
| | prior to randomization through Day 28; planned use of intra-articular steroid |
| | injections from the time of randomization through Day 28 |
| 12. | Allergy or significant reaction to any ingredient of the study drug, or to anesthetics |
| | or analgesics that may be used preoperatively or postoperatively |
| 13. | Current neurologic disorder, which could confound the assessment of pain (e.g., |
| | Parkinson's, Multiple Sclerosis) |
| 14. | Untreated or inadequately treated (in the opinion of the Investigator) active |
| | depression |
| 15. | MMSE score <24 at screening |
| 16. | Unstable mental condition which would prevent the patient from understanding the |
| | nature and scope of the study and/or evidence of an uncooperative attitude in the |
| | opinion of the Investigator; subjects diagnosed with schizophrenia, prescribed |
| | antipsychotic medications or MAOIs |
| 17. | History of alcohol-related complications within 1 year of randomization including, |
| | but not limited to, alcoholic withdrawal seizures, hallucinations, delirium tremens or |
| | detoxification treatment |
| 18. | Known or suspected history of illicit drug use within 1 year before randomization, |
| | or current or planned use of marijuana (including medical approved use) within 1 |
| | month before randomization and/or through the duration of the study |
| 19. | Any malignancy within the past year, with the exception of basal cell carcinoma or |
| | uncomplicated or stable skin cancers documented to not require further or immediate |
| | treatment |
| 20. | Women who are pregnant or nursing |
| 21. | Subjects engaged in pending or active litigation, or seeking disability compensation |
| | (including worker's compensation); subjects whose cases have been settled or finally |
| | decided are not excluded |
| 22. | Participation in a clinical trial with the last dose or intervention within 1 month of |
| | randomization, or planned participation in a clinical trial during this study |
| 23. | Previous participation in any study involving AYX1 Injection (with exposure to |
| | study drug) |
| 24. | Any condition that, in the opinion of the Investigator, could compromise the safety |
| | of the patient, the patient's ability to comply with study procedures, or the quality of |
| | the data |

Efficacy

Efficacy assessments included the following:
11-point Numerical Rating Scale (NRS) pain assessment at rest (after ˜30 minutes of rest), collected at screening, during the inpatient stay, and at follow-up visits by study staff
NRS pain assessment during the walk test, collected at screening (15 meter walk test), during the inpatient stay (5 meter walk test), and at follow-up visits (15 meter walk test) by study staff
NRS pain assessment for pain with rising from a seated position, and worst pain, least pain, and average pain over the last 24 hours, collected at screening by study staff, and by subjects every evening from Day 3 to Day 90 via eDiary
Collection of analgesic medication use through Day 90, recorded during the inpatient stay by study staff; recorded after discharge by subjects daily via eDiary

The efficacy assessments (pain at rest and the walk test) were performed by trained study staff during the inpatient period and at follow-up visits. Subjects were trained on the eDiary assessments at screening, on Day 1 prior to surgery (if needed), and prior to discharge from the hospital (or prior to the Day 3 evening assessment if still inpatient).

Primary Efficacy Endpoint:
Mean pain rating (NRS) with walking during the 15 meter walk test Day 7 to Day 28

Secondary Efficacy Endpoints:
Percentage of subjects with NRS pain score ≥4 during the 15 meter walk at Day 90
Mean pain rating (NRS) at rest Day 7 to Day 28
Time to achieve NRS pain score ≤3 for average pain
Time to achieve NRS pain score ≤3 for pain with rising from a seated position
Percentage of subjects with NRS pain score ≥3 at rest at Day 90
Total use of postoperative opioid medications (morphine equivalents) post-discharge to Day 90
Total use of postoperative opioid medications (morphine equivalents) 0-48 hours
Time to achieve NRS pain score ≤3 for worst pain

Additional Efficacy Endpoints:
Percentage of subjects with NRS pain score ≥3 during the 15 meter walk at Day 90
Percentage of subjects with NRS pain score ≥4 at rest at Day 90
Time to achieve NRS pain score ≤3 for least pain
Mean pain rating (NRS) with walking during the 5 meter walk test 24-48 hours
Mean pain rating at rest (NRS) 4-48 hours
Mean pain rating (NRS) with walking during the 15 meter walk test at Day 7
Mean pain rating (NRS) with walking during the 15 meter walk test at Day 28
Mean pain rating (NRS) at rest at Day 7
Mean pain rating (NRS) at rest at Day 28

NRS Pain Assessment at Rest

The 11-point Numerical Rating Scale (NRS) pain assessment at rest (for pain in the operated knee) was conducted after ˜30 minutes of rest at screening, at 4, 24, and 48 hours after completion of surgery (close of incision) during the inpatient stay, and at the follow-up visits on Days 7, 14, 21, 28, 42, 63, and 90.

NRS Pain Assessment with Rising from a Seated Position and Worst, Least, and Average Pain Over the Last 24 Hours

NRS pain assessment for pain with rising from a seated position, and worst pain, least pain, and average pain in the operated knee over the last 24 hours were collected at screening by study staff, and via eDiary by subjects every evening from Day 3 to Day 90. For pain with rising from a seated position, subjects were instructed to sit for at least 5 minutes prior to standing (therefore at screening, this assessment can be conducted after the pain at rest assessment). Subjects were instructed to use a chair without arms (or if the subject does not have a chair without arms, not to use the chair arms for assistance when standing), once they were able to safely stand without assistance.

Walk Test

The walk test was performed (with a walking frame or crutches as needed) at the following time points:
5 Meter walk test: at 24 and 48 hours after completion of surgery (close of incision) during the inpatient stay
15 Meter walk test: at screening and at the follow-up visits on Days 7, 14, 21, 28, 42, 63, and 90

Completion of the walk test and use of a walking aid were recorded by study staff. Knee immobilizers were not allowed during the walk test unless required for subject safety; use of a knee immobilizer during the walk test was documented. If the subject was not able to do the walk test or walk the entire distance, the reason was recorded (i.e., pain, fatigue, muscle weakness). The NRS pain score for pain in the operated knee during the walk was recorded by study staff after the walk is complete. If the subject could not complete the entire distance, the pain score for the portion of the walk that was completed was recorded.

Results

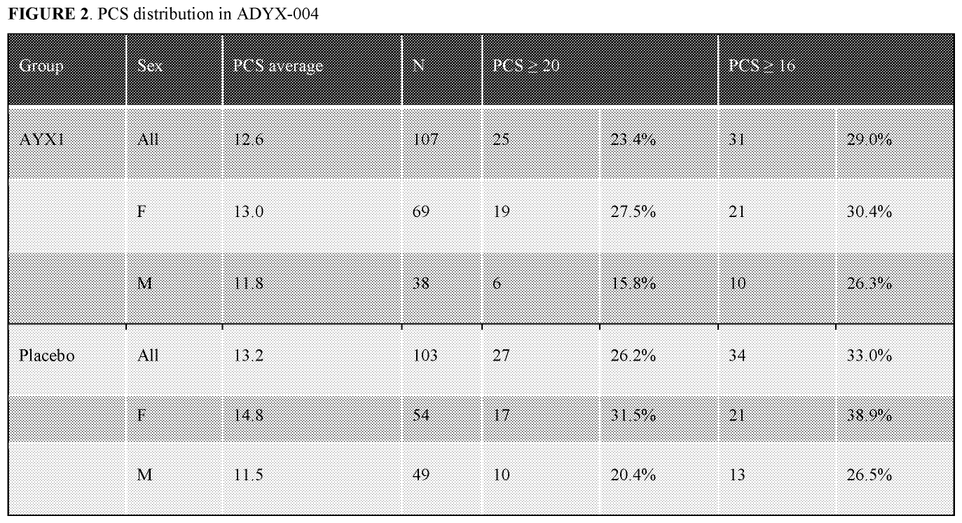

Since ADYX-004 was a larger study including a broader diversity of patients compared to prior AYX1 studies, it was decided to stratify the randomization of subjects by PCS score (≥20 vs. <20) to manage the expected downside risk of decreased efficacy of AYX1 in the high scoring subjects as predicted from literature reports for other analgesic interventions. FIG. 2 shows the patient distribution in ADYX-004 by baseline PCS scores.

When the data from ADYX-004 were unblinded, pre-specified endpoints in the study by PCS score and post-hoc analyses of the results indicated that the population responding best to AYX1 is the high PCS scoring population with score ≥20 as prespecified in the ADYX-004 study but also a broader patient population with PCS score ≥16, and not the low PCS scoring population.

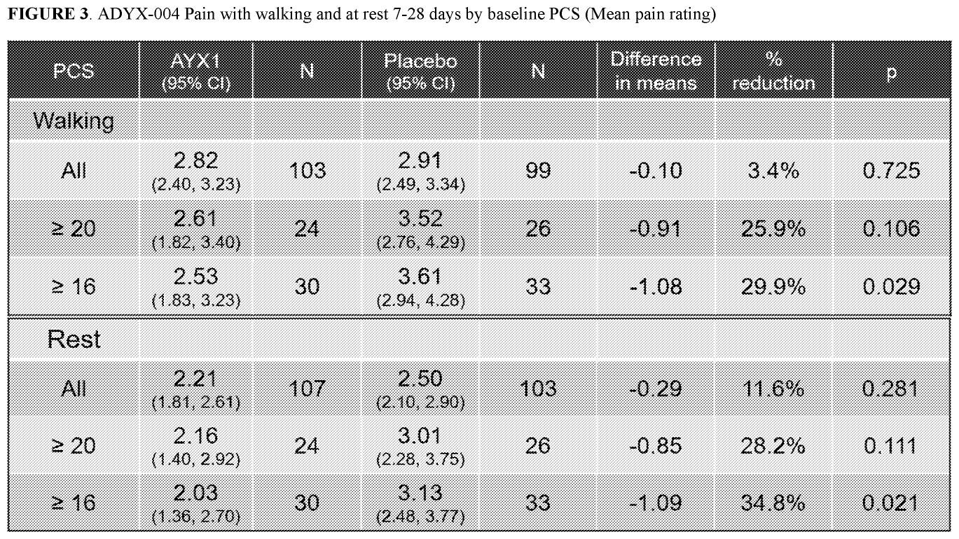

When the ADYX-004 results were filtered by PCS ≥20 or ≥16, AYX1 displayed a substantial treatment effect across multiple endpoints as discussed below. FIG. 3 shows the scores for pain with walking and at rest 7-28 days by baseline PCS (Mean pain rating). For patients who scored high on the PCS, AYX1 plus standard of care showed about 25% to 35% reduction in pain at rest and about 20% to 30% reduction in pain with walking (movement-evoked pain) compared to placebo plus standard of care (FIG. 3).

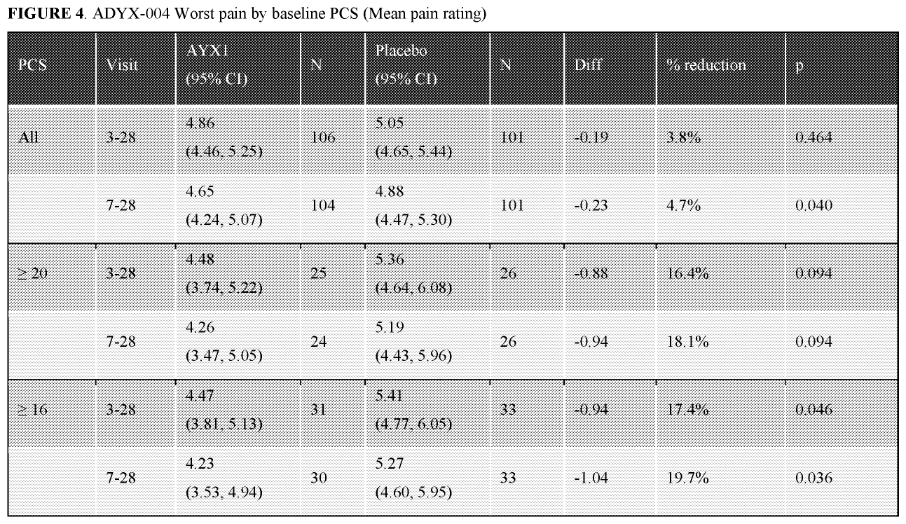

FIG. 4 shows the scores for worst pain by baseline PCS (Mean pain rating). AYX1 consistently reduced worst pain in patients who score high on the PCS (FIG. 4). The reduction in worst pain was by about 15% to 20% (FIG. 4).

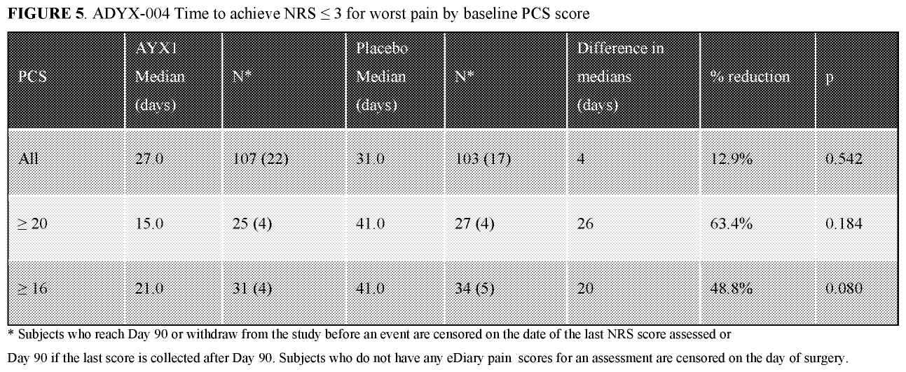

FIG. 5 shows time taken to achieve a change in the NRS score by ≤3 for worst pain by baseline PCS score. AYX1 improved the course of post-operative pain for patients who score high on the PCS by reducing the time taken achieve a change in the NRS score by ≤3. AYX1-treated patients with a PCS score of ≥20 showed a median reduction in time of 26 days to achieve NRS ≤3 for worst pain compared to the placebo-treated patients (FIG. 5). AYX1-treated patients with a PCS score of ≥16 showed a median reduction in time of 20 days to achieve NRS ≤3 for worst pain compared to the placebo-treated patients (FIG. 5).

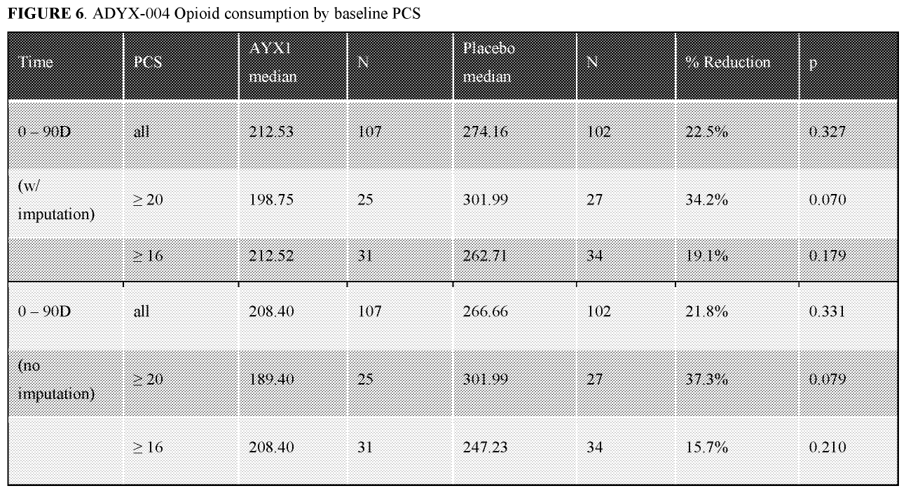

FIG. 6 shows opioid consumption by baseline PCS from day 0 through day 90 post-surgery. AYX1 treatment reduced opioid consumption for patients who score high on the PCS, a group normally associated with high opioid consumption and increased misuse potential. AYX1-treated patients with a PCS score of ≥20 showed about 30% to 40% reduction in opioid consumption compared to the placebo-treated patients (FIG. 6). AYX1-treated patients with a PCS score of ≥16 showed about 15% to 20% reduction in opioid consumption compared to the placebo-treated patients (FIG. 6).

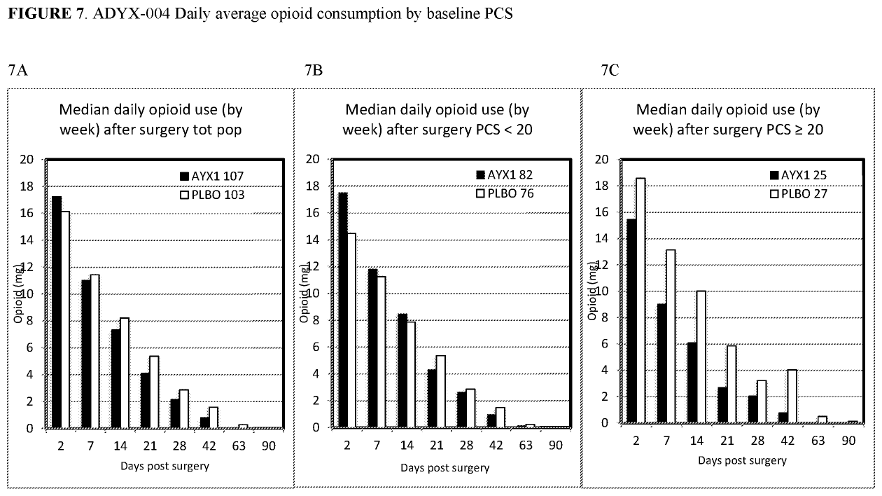

FIG. 7 shows daily average opioid consumption by baseline PCS. Panel 7A shows the median daily opioid use after surgery for the total population. Panel 7B shows the median daily opioid use after surgery for patients with a PCS score of <20. Panel 7C shows the median daily opioid use after surgery for patients with a PCS score of ≥20. Consistent reduction in opioid consumption for PCS ≥20 group manifested within 48 hours and was maintained over the duration of the study (panel 7C). The number after AYX1 or placebo (PLBO) showed in the figure legend of each panel shows the number of patients evaluated in that group. For example, AYX1 107 in the legend of panel 7A means that the total population evaluated in this figure included 107 patients, AYX1 82 in the legend of panel 7B means that the patient population with the PCS score of <20 evaluated in this panel included 82 patients, and so on.

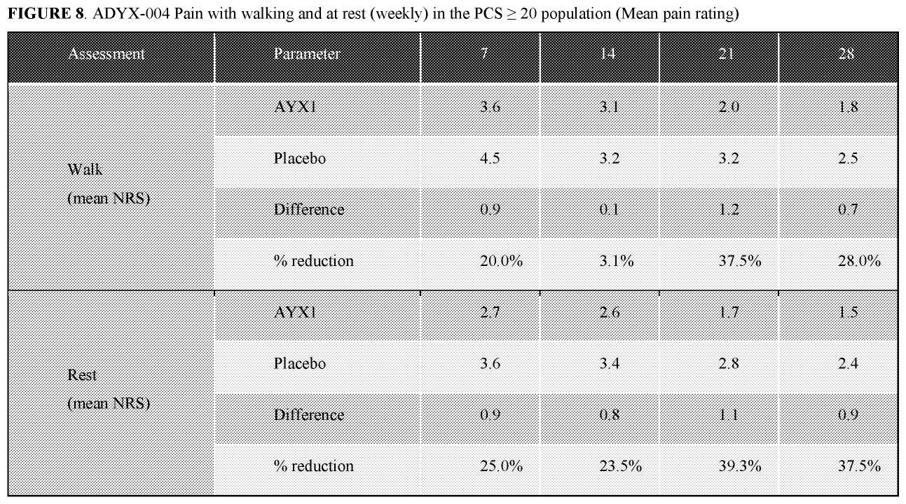

FIG. 8 shows the scores for pain with walking and at rest (weekly) in the PCS ≥20 population (Mean pain rating). AYX1 showed a consistent reduction in pain, both with walking and at rest, over the primary endpoint period of 7 to 28 days for this population.

### Example 2: Analysis of Patient Populations from Prior Clinical Trials ADYX-002 and ADYX-003

As noted above, in ADYX-004, patients were stratified based on their baseline PCS scores. Adynxx had collected the PCS score of all subjects in its prior clinical studies, ADYX-002 and AYDX-003, as it is a reported predictor of increased pain following surgery. In studies ADYX-002 and ADYX-003, PCS scores were collected for information only.

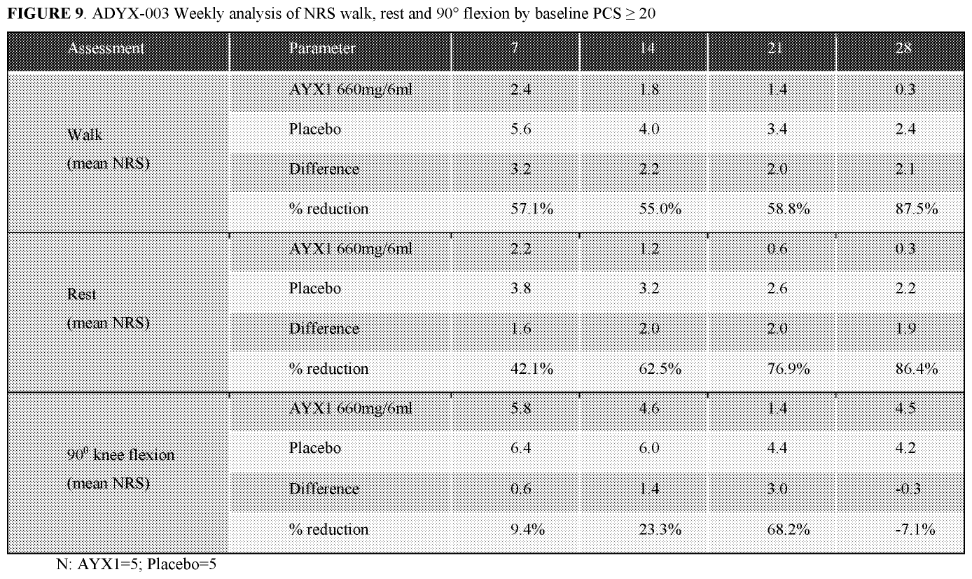

In view of the findings from ADYX-004 that the AYX1 treatment is particularly effective in the high PCS score patient population, Adynxx reanalyzed the results of ADYX-002 and ADYX-003 studies by comparing the high PCS scoring groups (≥20 or ≥16) compared to the lower scoring groups. The reanalysis of the data from ADYX-002 and ADYX-003 revealed that the relationship of higher PCS scores to higher efficacy of AYX1 was maintained across all the studies: when sorted by high PCS score (≥20 or ≥16), AYX1 displayed a much greater effect. FIGS. 9-13 show the data from ADYX-002, ADYX-003, or a combined data from ADYX-003 and ADYX-004. The data from these two clinical studies can be combined because the study protocols and endpoints were similar and the same 660 mg/6 mL dose were used in these two studies.

FIG. 9 shows a weekly analysis of the NRS scores for walk, rest and 90° flexion by baseline PCS ≥20 in the ADYX-003 clinical study. AYX1 treatment showed a substantial reduction in pain for all three end-points compared to the placebo-treated patient population.

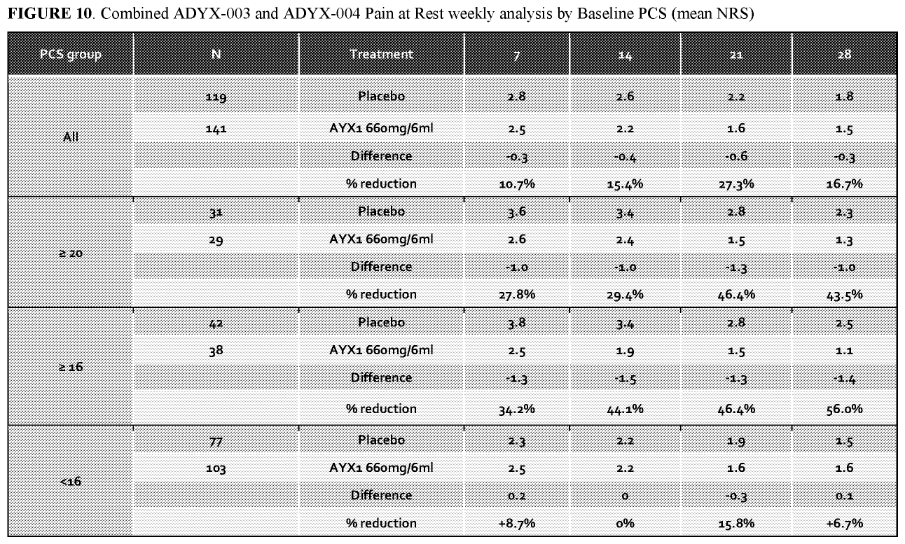

FIG. 10 shows the scores for pain with walking and pain at rest by baseline PCS (least square mean pain rating over 7-28 days) when the data from ADYX-003 and ADYX-004 were combined. The data from these two clinical studies can be combined because the study protocols and endpoints were similar and the same 660 mg/6 mL dose were used in these two studies.

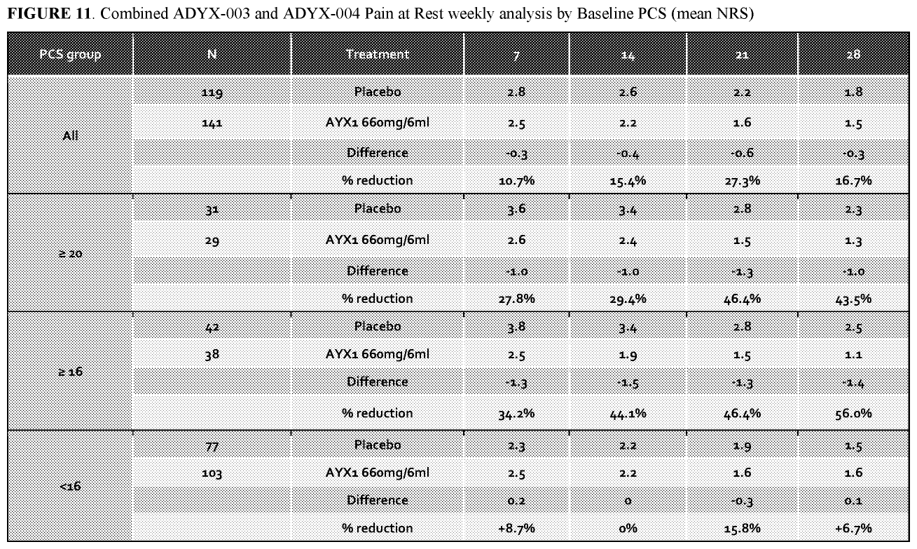

FIG. 11 shows a breakdown by time points for the weekly analysis of pain at rest shown in FIG. 10.

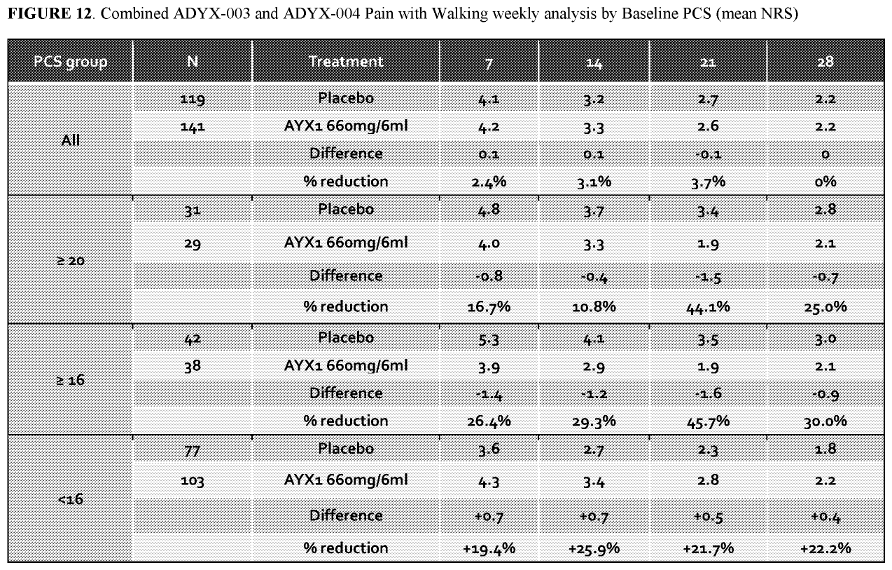

FIG. 12 shows a breakdown by time points for the weekly analysis of pain with walking shown in FIG. 10.

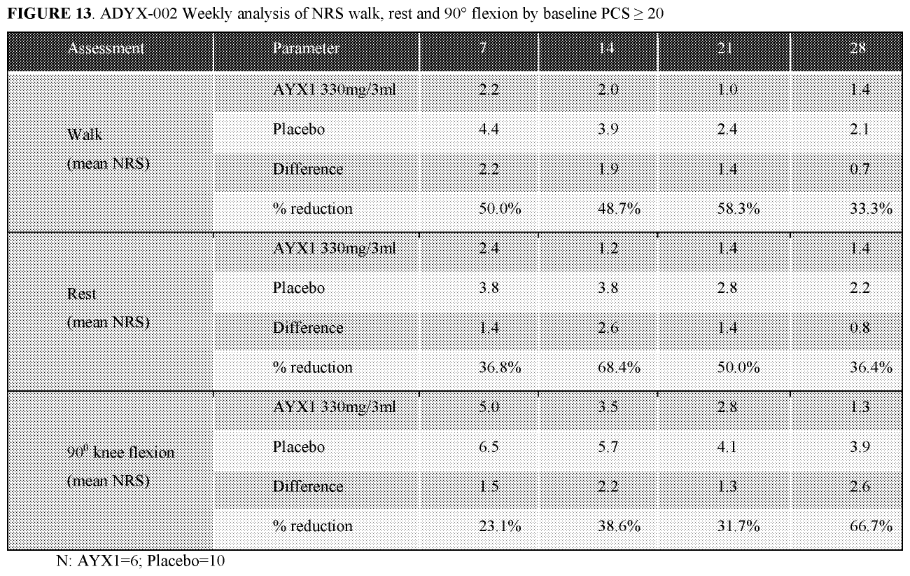

FIG. 13 shows a weekly analysis of the NRS scores for walk, rest and 90° flexion by baseline PCS ≥20 in the ADYX-002 clinical study. ADYX-002 study used 330 mg/3 mL dose of AYX1. AYX1 treatment showed a substantial reduction in pain for all three end-points compared to the placebo-treated patient population.

Summary of Findings in High PCS Populations Across Three Clinical Studies

PCS score has been collected in all Phase 2 clinical studies of AYX1 (ADYX-002, ADYX-003 and ADYX-004). All three studies independently show a strong and consistent treatment effect in patients who score high on the PCS. Treatment effect with patients who score high on the PCS was consistent across multiple endpoints and forms of data collection (in-clinic visits and E-diary) and applies to both PCS ≥20 and PCS ≥16 (both are considered cutoffs for high scores on the PCS).

Analysis of AYX1 effects by baseline PCS across all three Phase 2 studies shows a strong and consistent treatment effect for high catastrophizing subjects.

Specifically combining the ADYX-003 and ADYX-004 populations during the identical design period of 7-28 days and for the 660 mg/6 mL AYX1 dose revealed low estimated p values for the endpoints when cutoffs of ≥16 or ≥20 are used (FIGS. 10-12). The effect of AYX1 is similar in the ≥16 and ≥20 PCS groups, with the ≥16 PCS group capturing a wider range of the surgical population (33% vs 25%). Analysis of subjects below the score of 16 on the PCS revealed a lack of differentiation from placebo (and high estimated p values) despite a larger sample size.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### NUMBERED EMBODIMENTS OF THE DISCLOSURE

Notwithstanding the appended claims, the disclosure sets forth the following numbered embodiments:

1. A method for treating or preventing pain in a patient, said patient having a high pain catastrophizing scale (PCS) score, comprising: administering an oligonucleotide inhibitor of a transcription factor to said patient.

2. The method of embodiment 1, wherein said patient has a PCS score of 16 or greater.

3. The method of embodiment 1 or 2, wherein the oligonucleotide inhibitor is an oligonucleotide decoy comprising one or more transcription factor binding sites.

4. The method of any one of embodiments 1-3, wherein the transcription factor is Early Growth Response protein 1 (EGR1).

5. The method of any one of embodiments 1-4, wherein the oligonucleotide inhibitor is an oligonucleotide decoy comprising a nucleic acid sequence comprising a sense strand having a sequence selected from SEQ ID NOs: 1-53.

6. The method of embodiment 5, wherein the oligonucleotide decoy comprises an antisense strand having a sequence that is fully complementary to the sequence selected from SEQ ID NOs: 1-53.

7. The method of any one of embodiments 1-4, wherein the oligonucleotide inhibitor is an oligonucleotide decoy comprising a sequence selected from the group consisting of: (a) SEQ ID NOs: 1-53; (b) a sequence that is at least 90% identical to the sequence selected from SEQ ID NOs: 1-53; (c) a sequence that is at least 85% identical to the sequence selected from SEQ ID NOs: 1-53; and (d) a sequence that is at least 80% identical to the sequence selected from SEQ ID NOs: 1-53.

8. The method of any one of embodiments 1-7, wherein the oligonucleotide inhibitor is an oligonucleotide decoy comprising a nucleic acid sequence comprising a sense strand of 5′-GTATGCGTGGGCGGTGGGCGTAG-3′ (SEQ ID NO: 42).

9. The method of embodiment 8, wherein the oligonucleotide decoy comprises an antisense strand of 3′-CATACGCACCCGCCACCCGCATC-5′.

10. The method of any one of embodiments 1-7, wherein the oligonucleotide inhibitor is brivoligide (AYX1).

11. The method of any one of embodiments 1-10, for perioperative pain treatment or prevention in said patient.

12. The method of any one of embodiments 1-11, for post-operative pain treatment or prevention in said patient.

13. The method of any one of embodiments 1-12, wherein said patient experiences a clinically meaningful reduction in pain.

14. The method of any one of embodiments 1-13, wherein said patient experiences a clinically meaningful reduction in pain through at least day 28 post-surgery.

15. The method of any one of embodiments 1-14, wherein said patient experiences a clinically meaningful reduction in pain through at least day 42 post-surgery.

16. The method any one of embodiments 1-15, wherein said patient experiences a clinically meaningful reduction in pain through at least day 90 post-surgery.

17. The method any one of embodiments 13-16, wherein said reduction in pain is at least an additional 20% reduction in pain experienced by said patient as compared to a patient not administered the oligonucleotide inhibitor.

18. The method of any one of embodiments 1-12, wherein said patient experiences a clinically meaningful reduction in movement-evoked pain.

19. The method of any one of embodiments 1-12 and 18, wherein said patient experiences a clinically meaningful reduction in movement-evoked pain through at least day 28 post-surgery.

20. The method of any one of embodiments 1-12 and 18-19, wherein said patient experiences a clinically meaningful reduction in movement-evoked pain through at least day 42 post-surgery.

21. The method any one of embodiments 1-12 and 18-20, wherein said patient experiences a clinically meaningful reduction in movement-evoked pain through at least day 90 post-surgery.

22. The method any one of embodiments 18-21, wherein said reduction in movement-evoked pain experienced by said patient is at least an additional 20% reduction compared to a patient not administered the oligonucleotide inhibitor.

23. The method of any one of embodiments 1-12, wherein said patient experiences a clinically meaningful reduction in pain at rest.

24. The method of any one of embodiments 1-12 and 23, wherein said patient experiences a clinically meaningful reduction in pain at rest through at least day 28 post-surgery.

25. The method of any one of embodiments 1-12 and 23-24, wherein said patient experiences a clinically meaningful reduction in pain at rest through at least day 42 post-surgery.

26. The method of any one of embodiments 1-12 and 23-25, wherein said patient experiences a clinically meaningful reduction in pain at rest through at least day 90 post-surgery.

27. The method of any one of embodiments 23-26, wherein said reduction in pain at rest experienced by said patient is at least an additional 20% reduction compared to a patient not administered the oligonucleotide inhibitor.

28. The method of any one of embodiments 1-12, wherein said patient experiences a clinically meaningful reduction in movement-evoked pain from about day 7 post-surgery through at least day 28 post-surgery.

29. The method of any one of embodiments 1-12, wherein said patient experiences a clinically meaningful reduction in pain at rest from about day 7 post-surgery through at least day 28 post-surgery.

30. The method of any one of embodiments 1-12, wherein said patient experiences a clinically meaningful reduction in movement-evoked pain from about day 7 post-surgery through at least day 42 post-surgery.

31. The method of any one of embodiments 1-12, wherein the patient experiences a clinically meaningful reduction in pain at rest from about day 7 post-surgery through at least day 42 post-surgery.

32. The method of any one of embodiments 1-12, wherein the patient experiences a clinically meaningful reduction in movement-evoked pain from about day 7 post-surgery through at least day 90 post-surgery.

33. The method of any one of embodiments 1-12, wherein the patient experiences a clinically meaningful reduction in pain at rest from about day 7 post-surgery through at least day 90 post-surgery.

34. The method of any one of embodiments 28-33, wherein said reduction in movement-evoked pain or pain at rest experienced by said patient is at least an additional 20% reduction compared to a patient not administered the oligonucleotide inhibitor.

35. The method of any one of embodiments 1-34, wherein opioid consumption by said patient is reduced compared to a patient not administered the oligonucleotide inhibitor.

36. The method of any one of embodiments 1-35, wherein opioid consumption by said patient from day 0 post-surgery through at least day 90 post-surgery is reduced compared to a patient not administered the oligonucleotide inhibitor.

37. The method of any one of embodiments 1-36, wherein daily average opioid consumption by said patient is reduced compared to a patient not administered the oligonucleotide inhibitor.

38. The method of any one of embodiments 35-37, wherein said reduction in opioid consumption by said patient is at least an additional 5%, 10%, 15%, 20%, 25%, 30%, 35%, 40%, 45%, or 50%, compared to a patient not administered the oligonucleotide inhibitor.

39. The method of any one of embodiments 1-12, wherein said patient experiences a reduction in movement-evoked pain, wherein said reduction in pain is at least a 0.5 to 1 point reduction in pain, as measured by an 11 point numerical rating scale, experienced by said patient as compared to a patient not administered the oligonucleotide inhibitor.

40. The method of any one of embodiments 1-12, wherein said patient experiences a reduction in pain when at rest, wherein said reduction in pain is at least a 0.5 to 1 point reduction in pain, as measured by an 11 point numerical rating scale, experienced by said patient as compared to a patient not administered the oligonucleotide inhibitor.

41. The method of any one of embodiments 1-12, wherein said patient experiences a reduction in movement-evoked pain from about day 7 post-surgery through at least day 28 post-surgery, wherein said reduction in pain is at least a 0.5 to 1 point reduction in pain, as measured by an 11 point numerical rating scale, experienced by said patient as compared to a patient not administered the oligonucleotide inhibitor.

42. The method of any one of embodiments 1-12, wherein said patient experiences a reduction in pain when at rest from about day 7 post-surgery through at least day 28 post-surgery, wherein said reduction in pain is at least a 0.5 to 1 point reduction in pain, as measured by an 11 point numerical rating scale, experienced by said patient as compared to a patient not administered the oligonucleotide inhibitor.

43. The method of any one of embodiments 1-12, wherein said patient experiences a reduction in movement-evoked pain from about day 7 post-surgery through at least day 42 post-surgery, wherein said reduction in pain is at least a 0.5 to 1 point reduction in pain, as measured by an 11 point numerical rating scale, experienced by said patient as compared to a patient not administered the oligonucleotide inhibitor.

44. The method of any one of embodiments 1-12, wherein said patient experiences a reduction in pain at rest from about day 7 post-surgery through at least day 42 post-surgery, wherein said reduction in pain is at least a 0.5 to 1 point reduction in pain, as measured by an 11 point numerical rating scale, experienced by said patient as compared to a patient not administered the oligonucleotide inhibitor.

45. The method of any one of embodiments 1-12, wherein said patient experiences a reduction in movement-evoked pain from about day 7 post-surgery through at least day 90 post-surgery, wherein said reduction in pain is at least a 0.5 to 1 point reduction in pain, as measured by an 11 point numerical rating scale, experienced by said patient as compared to a patient not administered the oligonucleotide inhibitor.

46. The method of any one of embodiments 1-12, wherein said patient experiences a reduction in pain at rest from about day 7 post-surgery through at least day 90 post-surgery, wherein said reduction in pain is at least a 0.5 to 1 point reduction in pain, as measured by an 11 point numerical rating scale, experienced by said patient as compared to a patient not administered the oligonucleotide inhibitor.

47. The method of any one of embodiments 39-46, wherein time taken to achieve said reduction in pain by said patient is about 15 to 30 days less compared to a patient not administered the oligonucleotide inhibitor.

48. The method of any one of embodiments 1-12, wherein said patient experiences a reduction in movement-evoked pain, wherein said reduction in pain is at least an additional 20% reduction in pain, as measured by an 11 point numerical rating scale, experienced by said patient as compared to a patient not administered the oligonucleotide inhibitor.

49. The method of any one of embodiments 1-12, wherein said patient experiences a reduction in pain when at rest, wherein said reduction in pain is at least an additional 20% reduction in pain, as measured by an 11 point numerical rating scale, experienced by said patient as compared to a patient not administered the oligonucleotide inhibitor.

50. The method of any one of embodiments 1-12, wherein said patient experiences a reduction in movement-evoked pain from about day 7 post-surgery through at least day 28 post-surgery, wherein said reduction in pain is at least an additional 20% reduction in pain, as measured by an 11 point numerical rating scale, experienced by said patient as compared to a patient not administered the oligonucleotide inhibitor.

51. The method of any one of embodiments 1-12, wherein said patient experiences a reduction in pain when at rest from about day 7 post-surgery through at least day 28 post-surgery, wherein said reduction in pain is at least an additional 20% reduction in pain, as measured by an 11 point numerical rating scale, experienced by said patient as compared to a patient not administered the oligonucleotide inhibitor.

52. The method of any one of embodiments 1-12, wherein said patient experiences a reduction in movement-evoked pain from about day 7 post-surgery through at least day 42 post-surgery, wherein said reduction in pain is at least an additional 20% reduction in pain, as measured by an 11 point numerical rating scale, experienced by said patient as compared to a patient not administered the oligonucleotide inhibitor.

53. The method of any one of embodiments 1-12, wherein said patient experiences a reduction in pain at rest from about day 7 post-surgery through at least day 42 post-surgery, wherein said reduction in pain is at least an additional 20% reduction in pain, as measured by an 11 point numerical rating scale, experienced by said patient as compared to a patient not administered the oligonucleotide inhibitor.

54. The method of any one of embodiments 1-12, wherein said patient experiences a reduction in movement-evoked pain from about day 7 post-surgery through at least day 90 post-surgery, wherein said reduction in pain is at least an additional 20% reduction in pain, as measured by an 11 point numerical rating scale, experienced by said patient as compared to a patient not administered the oligonucleotide inhibitor.

55. The method of any one of embodiments 1-12, wherein said patient experiences a reduction in pain at rest from about day 7 post-surgery through at least day 90 post-surgery, wherein said reduction in pain is at least an additional 20% reduction in pain, as measured by an 11 point numerical rating scale, experienced by said patient as compared to a patient not administered the oligonucleotide inhibitor.

56. The method of any one of embodiments 48-55, wherein time taken to achieve said reduction in pain by said patient is about 15 to 30 days less compared to a patient not administered the oligonucleotide inhibitor.

57. The method of any one of embodiments 1-56, wherein the oligonucleotide inhibitor is administered to said patient at a concentration of about 110 mg/mL±25%.

58. The method of any one of embodiments 1-56, wherein the oligonucleotide inhibitor is administered to said patient at a concentration from about 660 mg/6 mL to less than about 1100 mg/10 mL.

59. The method of any one of embodiments 1-56, wherein the oligonucleotide inhibitor is administered to said patient at a concentration of less than about 1100 mg/10 mL.

60. The method of any one of embodiments 1-56, wherein the oligonucleotide inhibitor is administered to said patient at a concentration from about 500 mg/5 mL to about 700 mg/7 mL.

61. The method of any one of embodiments 1-56, wherein the oligonucleotide inhibitor is administered to said patient at a concentration from about 330 mg/3 mL to about 660 mg/6 mL.

62. The method of any one of embodiments 1-56, wherein the oligonucleotide inhibitor is administered to said patient at a concentration of about 660 mg/6 mL±25%.

63. The method of any one of embodiments 1-56, wherein the oligonucleotide inhibitor is administered to said patient at a concentration of about 660 mg/6 mL.

64. A method for treating or preventing pain in a patient, said patient having a high pain catastrophizing scale score, comprising: administering brivoligide to said patient.

65. The method of embodiment 64, for perioperative pain treatment or prevention in said patient.

66. A method for treating or preventing pain in a patient, said patient having a high pain catastrophizing scale score, comprising: administering an oligonucleotide decoy to said patient, wherein the oligonucleotide decoy comprises a nucleic acid sequence comprising a sense strand of 5′-GTATGCGTGGGCGGTGGGCGTAG-3′ and antisense strand of 3′-CATACGCACCCGCCACCCGCATC-5′.

67. A method for treating or preventing pain in a patient, said patient having a high pain catastrophizing scale score, comprising: administering an oligonucleotide decoy to said patient, wherein the oligonucleotide decoy comprises SEQ ID NO: 42.

68. A method for treating or preventing pain in a patient, said patient having a high pain catastrophizing scale score, comprising: administering an oligonucleotide decoy to said patient, wherein the oligonucleotide decoy has one or more EGR1 transcription factor binding sites.

69. A method for treating or preventing pain in a patient that is a member of a patient population that is often poorly-responsive to pain treatments, comprising: administering brivoligide to at least one member of said patient population.

70. The method of embodiment 69, for perioperative pain treatment or prevention in said patient.

71. A method for treating or preventing pain in a patient that is a member of a patient population that is often poorly-responsive to pain treatments, comprising: administering an oligonucleotide decoy to at least one member of said patient population, wherein the oligonucleotide decoy comprises a nucleic acid sequence comprising a sense strand of 5′-GTATGCGTGGGCGGTGGGCGTAG-3′ and antisense strand of 3′-CATACGCACCCGCCACCCGCATC-5′.

72. A method for treating or preventing pain in a patient that is a member of a patient population that is often poorly-responsive to pain treatments, comprising: administering an oligonucleotide decoy to at least one member of said patient population, wherein the oligonucleotide decoy comprises SEQ ID NO: 42.

73. A method for treating or preventing pain in a patient that is a member of a patient population that is often poorly-responsive to pain treatments, comprising: administering an oligonucleotide decoy to at least one member of said patient population, wherein the oligonucleotide decoy has one or more EGR1 transcription factor binding sites.

74. The method of any one of embodiments 1-63, wherein the oligonucleotide inhibitor is an oligonucleotide decoy comprising a sequence selected from the group consisting of: (a) the sequence of SEQ ID NO.: 42; (b) a sequence that is at least 90% identical with SEQ ID NO.: 42; (c) a sequence that is at least 85% identical with SEQ ID NO.: 42; or (d) a sequence that is at least 80% identical with SEQ ID NO.: 42.

1. A method for treating or preventing pain in a patient, said patient having a high pain catastrophizing scale (PCS) score, comprising: administering an oligonucleotide inhibitor of a transcription factor to said patient.

2. The method of claim 1, wherein said patient has a PCS score of 16 or greater.

3. The method of claim 1, wherein the oligonucleotide inhibitor is
a) an oligonucleotide decoy comprising one or more transcription factor binding sites; or
b) brivoligide (AYX1).

4. The method of claim 1, wherein the transcription factor is Early Growth Response protein 1 (EGR1).

5. The method of claim 1, wherein the oligonucleotide inhibitor is an oligonucleotide decoy comprising a nucleic acid sequence comprising a sense strand having a sequence selected from SEQ ID NOs: 1-53.

6. The method of claim 5, wherein the oligonucleotide decoy comprises an antisense strand having a sequence that is fully complementary to the sequence selected from SEQ ID NOs: 1-53.

7. The method of claim 1, wherein the oligonucleotide inhibitor is an oligonucleotide decoy comprising a sequence selected from the group consisting of: (a) SEQ ID NOs: 1-53; (b) a sequence that is at least 90% identical to the sequence selected from SEQ ID NOs: 1-53; (c) a sequence that is at least 85% identical to the sequence selected from SEQ ID NOs: 1-53; and (d) a sequence that is at least 80% identical to the sequence selected from SEQ ID NOs: 1-53.

8. The method of claim 1, wherein the oligonucleotide inhibitor is an oligonucleotide decoy comprising a nucleic acid sequence comprising a sense strand of 5′-GTATGCGTGGGCGGTGGGCGTAG-3′ (SEQ ID NO: 42).

9. The method of claim 8, wherein the oligonucleotide decoy comprises an antisense strand of 3′-CATACGCACCCGCCACCCGCATC-5′.

10. (canceled)

11. The method of claim 1, wherein administration of the oligonucleotide inhibitor of a transcription factor to said patient
a) treats or prevents perioperative pain in said patient
b) treats or prevents post-operative pain in said patient
c) results in a clinically meaningful reduction in pain in said patient
d) experiences a clinically meaningful reduction in movement-evoked pain; or
e) results in a clinically meaningful reduction in pain at rest.

12-13. (canceled)

14. The method of claim 11, wherein said patient experiences a clinically meaningful reduction in pain through
a) at least day 28 post-surgery;
b) at least day 42 post-surgery; or
c) at least day 90 post-surgery.

15-16. (canceled)

17. The method of claim 11, wherein said reduction in pain is at least an additional 20% reduction in pain experienced by said patient as compared to a patient not administered the oligonucleotide inhibitor.

18.-34. (canceled)

35. The method of claim 1, wherein opioid consumption by said patient is reduced compared to a patient not administered the oligonucleotide inhibitor.

36-56. (canceled)

57. The method of claim 1, wherein the oligonucleotide inhibitor is administered to said patient at a concentration of about 110 mg/mL±25%.

58. The method of claim 1, wherein the oligonucleotide inhibitor is administered to said patient at a concentration from about 660 mg/6 mL to less than about 1100 mg/10 mL.

59. The method of claim 58, wherein the oligonucleotide inhibitor is administered to said patient at a concentration of less than about 1100 mg/10 mL.

60. (canceled)

61. The method of claim 59, wherein the oligonucleotide inhibitor is administered to said patient at a concentration from about 330 mg/3 mL to about 660 mg/6 mL.

62. The method of claim 61, wherein the oligonucleotide inhibitor is administered to said patient at a concentration of about 660 mg/6 mL±25%.

63. The method of claim 62, wherein the oligonucleotide inhibitor is administered to said patient at a concentration of about 660 mg/6 mL.

64. The method claim 1, wherein the oligonucleotide inhibitor is an oligonucleotide decoy comprising a sequence selected from the group consisting of: (a) the sequence of SEQ ID NO.: 42; (b) a sequence that is at least 90% identical with SEQ ID NO.: 42; (c) a sequence that is at least 85% identical with SEQ ID NO.: 42; or (d) a sequence that is at least 80% identical with SEQ ID NO.: 42.


SEQUENCE LISTING (54 sequences)

SEQ ID NO: 1 - DNA, 31 bp, Artificial Sequence
  oligonucleotide decoy
ggcttatgcaaattcgaatgcaaatttgtcg

SEQ ID NO: 2 - DNA, 36 bp, Artificial Sequence
  oligonucleotide decoy
ctaagcccacgtgaccattggccaggtgaccagatc

SEQ ID NO: 3 - DNA, 34 bp, Artificial Sequence
  oligonucleotide decoy
gttatgcgtgggcgataatgcgggggcgttatag

SEQ ID NO: 4 - DNA, 29 bp, Artificial Sequence
  oligonucleotide decoy
gcctccctgagctcattgacgtatctcgg

SEQ ID NO: 5 - DNA, 30 bp, Artificial Sequence
  oligonucleotide decoy
cgaatatgactgagaatgactcagatttgc

SEQ ID NO: 6 - DNA, 37 bp, Artificial Sequence
  oligonucleotide decoy
ggttctatgattttggaatcggattgtgcaaagaagc

SEQ ID NO: 7 - DNA, 34 bp, Artificial Sequence
  oligonucleotide decoy
gcttcaggatgtccatattaggagatcttgttcg

SEQ ID NO: 8 - DNA, 34 bp, Artificial Sequence
  oligonucleotide decoy
ggccacaggatgtaggatgtccatattaggatgc

SEQ ID NO: 9 - DNA, 35 bp, Artificial Sequence
  oligonucleotide decoy
gttctctaaaaataaaaggctaaaaataaaagtcg

SEQ ID NO: 10 - DNA, 30 bp, Artificial Sequence
  oligonucleotide decoy
attaggggcggggtccggggcggggtatta

SEQ ID NO: 11 - DNA, 35 bp, Artificial Sequence
  oligonucleotide decoy
gttatggcggggcggggcggggccgggcggtttac

SEQ ID NO: 12 - DNA, 29 bp, Artificial Sequence
  oligonucleotide decoy
ggcaatgtggttttagtgtggttttacgg

SEQ ID NO: 13 - DNA, 36 bp, Artificial Sequence
  oligonucleotide decoy
gccgtttggggtcatagaaccacaggaaccacacgg

SEQ ID NO: 14 - DNA, 32 bp, Artificial Sequence
  oligonucleotide decoy
cattgcccggaaatggaccggatgtaatttcc

SEQ ID NO: 15 - DNA, 40 bp, Artificial Sequence
  oligonucleotide decoy
gttcttggaaaataaatggaaaatagtggaaaataagtcg

SEQ ID NO: 16 - DNA, 33 bp, Artificial Sequence
  oligonucleotide decoy
cgttcccacttcctgcgaccacttcctgccggg

SEQ ID NO: 17 - DNA, 33 bp, Artificial Sequence
  oligonucleotide decoy
ctgcacctataaatggcctataaatggggatgc

SEQ ID NO: 18 - DNA, 33 bp, Artificial Sequence
  oligonucleotide decoy
gcttatttcgcggaaggtttcccggaagtggcg

SEQ ID NO: 19 - DNA, 31 bp, Artificial Sequence
  oligonucleotide decoy
gctgtgccttatctctttgggataactggcg

SEQ ID NO: 20 - DNA, 32 bp, Artificial Sequence
  oligonucleotide decoy
gcttaatgaataagaggaaaaatgcatgctgg

SEQ ID NO: 21 - DNA, 33 bp, Artificial Sequence
  oligonucleotide decoy
gttctgagattgcacgatgagatttcacagtcg

SEQ ID NO: 22 - DNA, 32 bp, Artificial Sequence
  oligonucleotide decoy
gtcccgcataaataatggcatccttaatcgcg

SEQ ID NO: 23 - DNA, 36 bp, Artificial Sequence
  oligonucleotide decoy
gtgcaggcaagagtagagacaggcaagagtagatgc

SEQ ID NO: 24 - DNA, 25 bp, Artificial Sequence
  oligonucleotide decoy
ccgccaataattaattattaaggcc

SEQ ID NO: 25 - DNA, 35 bp, Artificial Sequence
  oligonucleotide decoy
gcttcgttccatttccggtctcggtttccccattc

SEQ ID NO: 26 - DNA, 34 bp, Artificial Sequence
  oligonucleotide decoy
gctgctgtggaatatcgacctgtggaatatcgtg

SEQ ID NO: 27 - DNA, 37 bp, Artificial Sequence
  oligonucleotide decoy
gccgtataaatgtgctataaaagttttaagaccgtgc

SEQ ID NO: 28 - DNA, 29 bp, Artificial Sequence
  oligonucleotide decoy
gccgtataaatgtgctataaaagccgtgc

SEQ ID NO: 29 - DNA, 26 bp, Artificial Sequence
  oligonucleotide decoy
atgctgcgcttttctccaatctgcgg

SEQ ID NO: 30 - DNA, 40 bp, Artificial Sequence
  oligonucleotide decoy
cgttctccgattggtcacggactctccgattggtcacggc

SEQ ID NO: 31 - DNA, 28 bp, Artificial Sequence
  oligonucleotide decoy
gcgcaccccagcctggctcacccacgcg

SEQ ID NO: 32 - DNA, 37 bp, Artificial Sequence
  oligonucleotide decoy
gatcctttgcctccttcgatcctttgcctccttcaag

SEQ ID NO: 33 - DNA, 29 bp, Artificial Sequence
  oligonucleotide decoy
ggtgtttgggagagctttgggaggatacg

SEQ ID NO: 34 - DNA, 36 bp, Artificial Sequence
  oligonucleotide decoy
gctaatcactcagcatttcggtgagggaagtgaaag

SEQ ID NO: 35 - DNA, 51 bp, Artificial Sequence
  oligonucleotide decoy
cctttcagcaccacggacagcgccagcttcagcaccacggacagcgcctcg

SEQ ID NO: 36 - DNA, 36 bp, Artificial Sequence
  oligonucleotide decoy
ggatcgaacatggagtcagtgagaaatcaggatcgg

SEQ ID NO: 37 - DNA, 35 bp, Artificial Sequence
  oligonucleotide decoy
ggatcgaagccggagtcaaggaggcccctgatcgg

SEQ ID NO: 38 - DNA, 39 bp, Artificial Sequence
  oligonucleotide decoy
ccgaaaggacaaaggtcaagtcgaaaggacaaaggtcag

SEQ ID NO: 39 - DNA, 35 bp, Artificial Sequence
  oligonucleotide decoy
cgggagaaaattcgggaacgttcaagaattgtcgg

SEQ ID NO: 40 - DNA, 34 bp, Artificial Sequence
  oligonucleotide decoy
gttatgcgtgggcgtagatgcgggggcgttatag

SEQ ID NO: 41 - DNA, 16 bp, Artificial Sequence
  oligonucleotide decoy
gatgcgtgggcgtagg

SEQ ID NO: 42 - DNA, 23 bp, Artificial Sequence
  oligonucleotide decoy
gtatgcgtgggcggtgggcgtag

SEQ ID NO: 43 - DNA, 30 bp, Artificial Sequence
  oligonucleotide decoy
gttatgcgtttgtagatgctttcgttatag

SEQ ID NO: 44 - DNA, 20 bp, Artificial Sequence
  oligonucleotide decoy
gttatgcgtgggcgatatag

SEQ ID NO: 45 - DNA, 28 bp, Artificial Sequence
  oligonucleotide decoy
gatgcgtgggcgttgacgtggaaaatgc

SEQ ID NO: 46 - DNA, 30 bp, Artificial Sequence
  oligonucleotide decoy
ctatttcgaaacgatctacattggcataac

SEQ ID NO: 47 - DNA, 22 bp, Artificial Sequence
  oligonucleotide decoy
cgttcccacttcctgcgaccgg

SEQ ID NO: 48 - DNA, 26 bp, Artificial Sequence
  oligonucleotide decoy
gggtgaaggcaagagtagagcggcgg

SEQ ID NO: 49 - DNA, 21 bp, Artificial Sequence
  oligonucleotide decoy
cgttctccgattggtcacgcg

SEQ ID NO: 50 - DNA, 26 bp, Artificial Sequence
  oligonucleotide decoy
gtactccctttgcctccttcaaccgg

SEQ ID NO: 51 - DNA, 31 bp, Artificial Sequence
  oligonucleotide decoy
ccttattcagcaccacggacagcgccattcg

SEQ ID NO: 52 - DNA, 23 bp, Artificial Sequence
  oligonucleotide decoy
gcgaaaggacaaaggtcaggcgg

SEQ ID NO: 53 - DNA, 25 bp, Artificial Sequence
  oligonucleotide decoy
ggcttgctgtggaatatcgatggtg

SEQ ID NO: 54 - DNA, 23 bp, Artificial Sequence
  oligonucleotide decoy
ctacgcccaccgcccacgcatac